(19) United States
(12) Patent Application Publication
(10) Pub. No.: US 2022/0149295 A1
(43) Pub. Date: May 12, 2022

(54) COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Youngkyoung JO, Suwon-si (KR); Dong Min KANG, Suwon-si (KR); Byungku KIM, Suwon-si (KR); Kipo JANG, Suwon-si (KR); Sung-Hyun JUNG, Suwon-si (KR); Ho Kuk JUNG, Suwon-si (KR); Dongyeong KIM, Suwon-si (KR); Namheon LEE, Suwon-si (KR); Mijin LEE, Suwon-si (KR); Sangshin LEE, Suwon-si (KR); Sangil LEE, Suwon-si (KR)

(21) Appl. No.: 17/516,778

(22) Filed: Nov. 2, 2021

(30) Foreign Application Priority Data
Nov. 9, 2020 (KR) 10-2020-0148810

(51) Int. Cl. H01L 51/00; C09K 11/02; C09K 11/06; C07D 403/04; C07D 307/93; C07D 403/10

(52) U.S. Cl. CPC H01L 51/0072; C09K 11/02; C09K 11/06; C07D 403/04; C07D 307/93; C07D 403/10; H01L 51/5016; H01L 51/0061; C09K 2211/185; C09K 2211/1029; C09K 2211/1007; H01L 51/0073; H01L 51/0067

(57) ABSTRACT

A composition for an organic optoelectronic device, an organic optoelectronic device including the same, and a display device, the composition including a first compound represented by Chemical Formula 1, and a second compound represented by Chemical Formula 2,

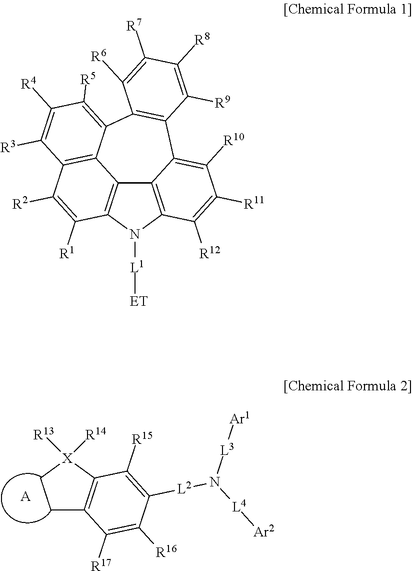

### CROSS-REFERENCE TO RELATED APPLICATION

This application claims priority to and the benefit of Korean Patent Application No. 10-2020-0148810 filed in the Korean Intellectual Property Office on Nov. 9, 2020, the entire contents of which are incorporated herein by reference.

### BACKGROUND

### 1. Field

Embodiments relate composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be largely divided into two types according to a principle of operation. One type includes a photoelectric device that generates electrical energy by separating excitons formed by light energy into electrons and holes, and transferring the electrons and holes to different electrodes, respectively and another type includes a light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing a composition for an organic optoelectronic device, the composition including a first compound represented by Chemical Formula 1, and a second compound represented by Chemical Formula 2,

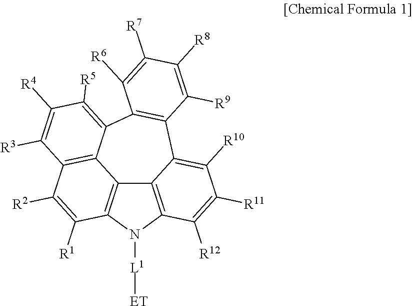

wherein, in Chemical Formula 1, R¹ to R¹² are each independently hydrogen, deuterium, a cyano group, halogen, a substituted or unsubstituted C1 to C30 alkyl group, or a substituted or unsubstituted C6 to C30 aryl group, L¹ is a single bond or a substituted or unsubstituted C6 to C30 arylene group, and ET is a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzofuranpyrimidinyl group, or a substituted or unsubstituted benzothiophenepyrimidinyl group;

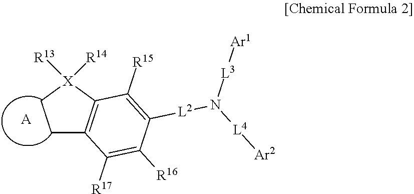

wherein, in Chemical Formula 2, X is C or Si, R¹³ and R¹⁴ are each independently a substituted or unsubstituted C1 to C30 alkyl group or a substituted or unsubstituted C6 to C30 aryl group, R¹⁵ to R¹⁷ are each independently hydrogen, deuterium, a substituted or unsubstituted C1 to C30 alkyl group, a substituted or unsubstituted C6 to C30 aryl group, or a substituted or unsubstituted C2 to C30 heterocyclic group, L² to L⁴ are each independently a single bond or a substituted or unsubstituted C6 to C30 arylene group, Ar¹ and Ar² are each independently a substituted or unsubstituted C6 to C30 aryl group, a substituted or unsubstituted dibenzofuranyl group, or a substituted or unsubstituted dibenzothiophenyl group, and A is a ring of Group I,

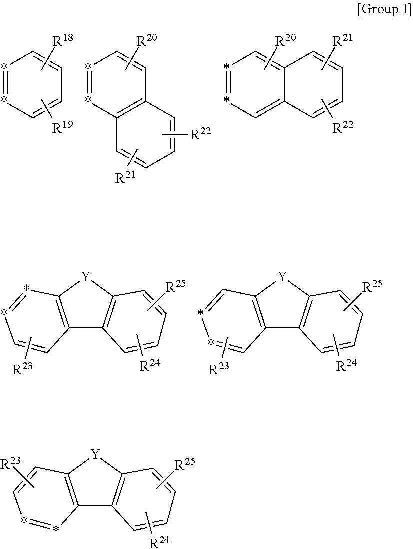

wherein, in Group I, each * is a linking carbon, Y is O or S, and R¹⁸ to R²⁵ are each independently hydrogen, deuterium, a substituted or unsubstituted C1 to C30 alkyl group, a substituted or unsubstituted C6 to C30 aryl group, or a substituted or unsubstituted C2 to C30 heterocyclic group.

The embodiments may be realized by providing an organic optoelectronic device including an anode and a cathode facing each other, and at least one organic layer between the anode and the cathode, wherein the at least one organic layer includes a light emitting layer, and the light emitting layer includes the composition for an organic optoelectronic device according to an embodiment.

The embodiments may be realized by providing a display device including the organic optoelectronic device according to an embodiment.

### BRIEF DESCRIPTION OF THE DRAWINGS

Features will be apparent to those of skill in the art by describing in detail exemplary embodiments with reference to the attached drawings in which:

FIGS. 1 to 4 are cross-sectional views OF organic light emitting diodes according to embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figures, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof. As used herein, the term “or” is not an exclusive term, e.g., “A or B” would include A, B, or A and B.

In one example, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In a specific example, the “substituted” refers to replacement of at least one hydrogen of a substituent or a compound by deuterium, a C1 to C20 alkyl group, a C6 to C30 aryl group, or a cyano group. In a specific example, the “substituted” refers to replacement of at least one hydrogen of a substituent or a compound by deuterium, a C1 to C5 alkyl group, a C6 to C18 aryl group, or a cyano group. In a specific example, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, when a definition is not otherwise provided, “hetero” refers to one including one to three heteroatoms selected from N, O, S, P, and Si, and remaining carbons in one functional group.

As used herein, “an aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzofuranpyrimidinyl group, a substituted or unsubstituted benzothiophenepyrimidinyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to an embodiment is described.

A composition for an organic optoelectronic device according to an embodiment may include, e.g., a first compound represented by Chemical Formula 1 and a second compound represented by Chemical Formula 2.

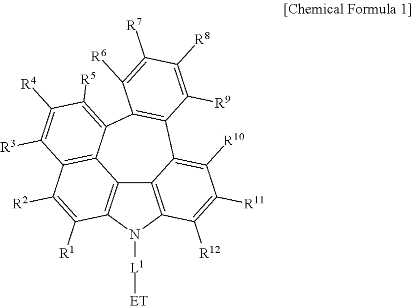

In Chemical Formula 1, R¹ to R¹² may each independently be or include, e.g., hydrogen, deuterium, a cyano group, halogen, a substituted or unsubstituted C1 to C30 alkyl group, or a substituted or unsubstituted C6 to C30 aryl group.

L¹ may be or may include, e.g., a single bond or a substituted or unsubstituted C6 to C30 arylene group.

ET may be or may include, e.g., a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzofuranpyrimidinyl group, or a substituted or unsubstituted benzothiophenepyrimidinyl group.

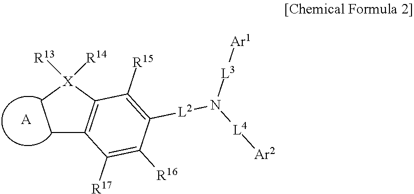

In Chemical Formula 2, X may be, e.g., C or Si.

R¹³ and R¹⁴ may each independently be or include, e.g., a substituted or unsubstituted C1 to C30 alkyl group or a substituted or unsubstituted C6 to C30 aryl group.

R¹⁵ to R¹⁷ may each independently be or include, e.g., hydrogen, deuterium, a substituted or unsubstituted C1 to C30 alkyl group, a substituted or unsubstituted C6 to C30 aryl group, or a substituted or unsubstituted C2 to C30 heterocyclic group.

L² to L⁴ may each independently be or include, e.g., a single bond or a substituted or unsubstituted C6 to C30 arylene group.

Ar¹ and Ar² may each independently be or include, e.g., a substituted or unsubstituted C6 to C30 aryl group, a substituted or unsubstituted dibenzofuranyl group, or a substituted or unsubstituted dibenzothiophenyl group.

A may be, e.g., a ring of Group I.

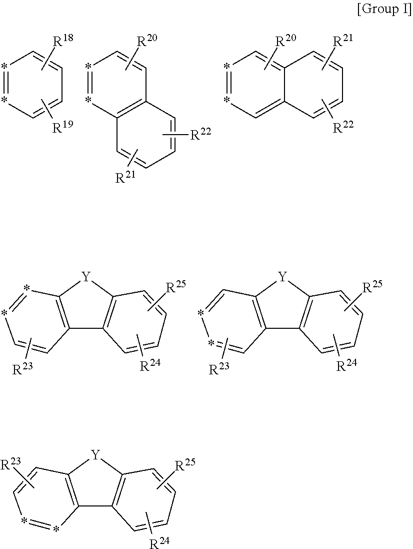

In Group I, * is a linking carbon. As used herein, the term “linking carbon” refers to a shared carbon at which fused rings are linked.

Y may be, e.g., O or S.

R¹⁸ to R²⁵ may each independently be or include, e.g., hydrogen, deuterium, a substituted or unsubstituted C1 to C30 alkyl group, a substituted or unsubstituted C6 to C30 aryl group, or a substituted or unsubstituted C2 to C30 heterocyclic group.

The first compound represented by Chemical Formula 1 may have a structure advantageous for pi-pi stacking by including a planar core, and may have a high glass transition temperature relative to molecular weight to have excellent thermal stability.

In an implementation, when applied to an organic light emitting diode together with the second compound represented by Chemical Formula 2, charge balance may be achieved to realize a long life-span.

The second compound may have a structure in which a fluorene or a fused fluorene is substituted with an amine group, the HOMO electron cloud may be expanded from amine to fluorene or fused fluorene to have high HOMO energy, and thus the second compound may have excellent hole injection and transport characteristics.

In addition, the amine may be substituted at the 2-position of the fluorene or fused fluorene, and planarity of the molecule may be maintained and a deposition temperature thereof may be increased, thereby improving thermal stability during manufacture of a device.

In an implementation, ET of Chemical Formula 1 may be, e.g., a substituted or unsubstituted pyrimidinyl group, a substituted or unsubstituted triazinyl group, a substituted or unsubstituted quinazolinyl group, a substituted or unsubstituted quinoxalinyl group, a substituted or unsubstituted benzofuranpyrimidinyl group, or a substituted or unsubstituted benzothiophenepyrimidinyl group.

In an implementation, ET may be, e.g., a substituted or unsubstituted pyrimidinyl group, a substituted or unsubstituted triazinyl group, a substituted or unsubstituted quinazolinyl group, or a substituted or unsubstituted quinoxalinyl group.

In an implementation, when ET is substituted, the substituent may include a phenyl group unsubstituted or substituted with a C6 to C12 aryl group, a biphenyl group unsubstituted or substituted with a C6 to C12 aryl group, a naphthyl group unsubstituted or substituted with a C6 to C12 aryl group, an anthracenyl group unsubstituted or substituted with a C6 to C12 aryl group, a fluorenyl group unsubstituted or substituted with a C6 to C12 aryl group, a dibenzofuranyl group unsubstituted or substituted with a C6 to C12 aryl group, a dibenzothiophenyl group unsubstituted or substituted with a C6 to C12 aryl group, or a dibenzosilolyl group unsubstituted or substituted with a C6 to C12 aryl group.

In an implementation, ET may be a group of Group II.

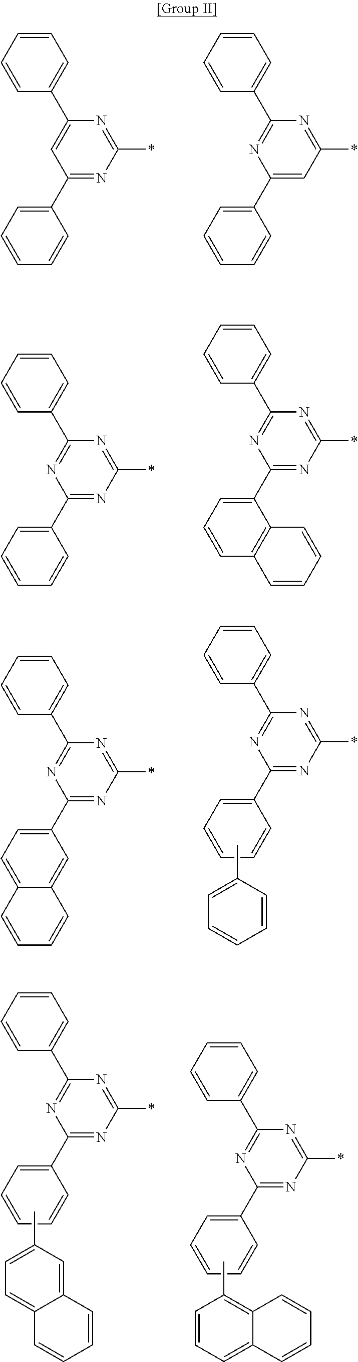

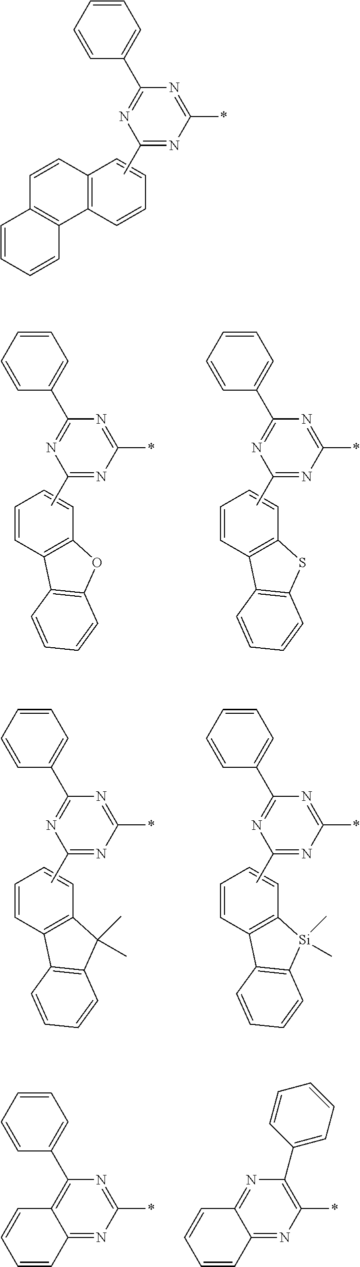

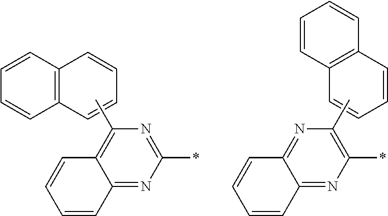

In Group II, * is a linking point (e.g., to L¹ or N of Chemical Formula 1).

In an implementation, ET may be, e.g., a substituted or unsubstituted triazinyl group or a substituted or unsubstituted quinoxalinyl group.

In an implementation, L¹ of Chemical Formula 1 may be, e.g., a single bond, a substituted or unsubstituted phenylene group, a substituted or unsubstituted biphenylene group, or a substituted or unsubstituted naphthylene group.

In an implementation, L¹ in Chemical Formula 1 may be, e.g., a single bond or an ortho-phenylene group.

In an implementation, R¹ to R¹² in Chemical Formula 1 may each independently be, e.g., hydrogen, deuterium, a cyano group, a halogen, a substituted or unsubstituted C1 to C10 alkyl group, a substituted or unsubstituted phenyl group, a substituted or unsubstituted naphthyl group, or a substituted or unsubstituted biphenyl group.

In an implementation, R¹ to R¹² in Chemical Formula 1 may each independently be, e.g., hydrogen, deuterium, a cyano group, a halogen, or a substituted or unsubstituted phenyl group.

In an implementation, the first compound may be, e.g., a compound of Group 1.

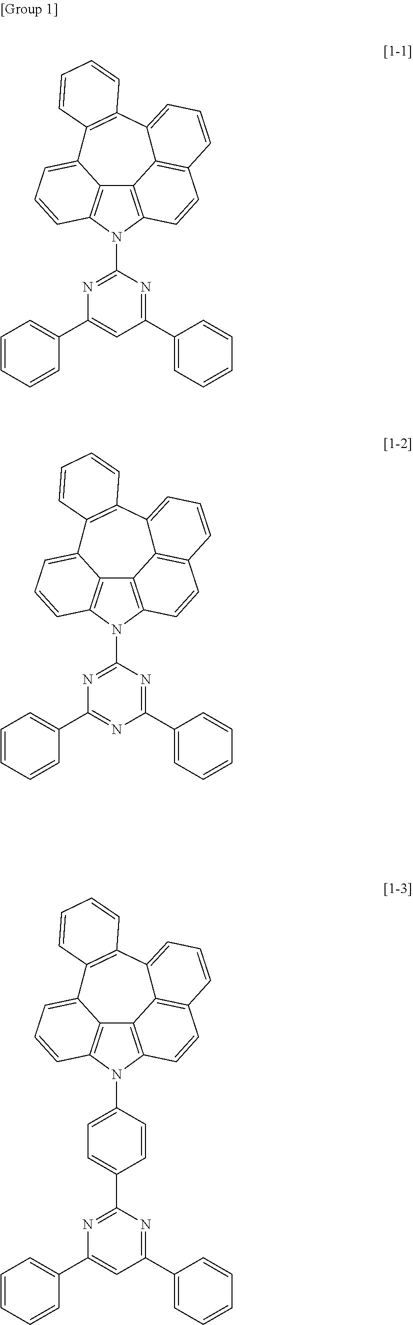 [SMILES: c1ccc(-c2cc(-c3ccccc3)nc(-n3c4cccc5c4c4c6c(cccc6ccc43)-c3ccccc3-5)n2)cc1 ; c1ccc(-c2nc(-c3ccccc3)nc(-n3c4cccc5c4c4c6c(cccc6ccc43)-c3ccccc3-5)n2)cc1 ; c1ccc(-c2cc(-c3ccccc3)nc(-c3ccc(-n4c5cccc6c5c5c7c(cccc7ccc54)-c4ccccc4-6)cc3)n2)cc1]

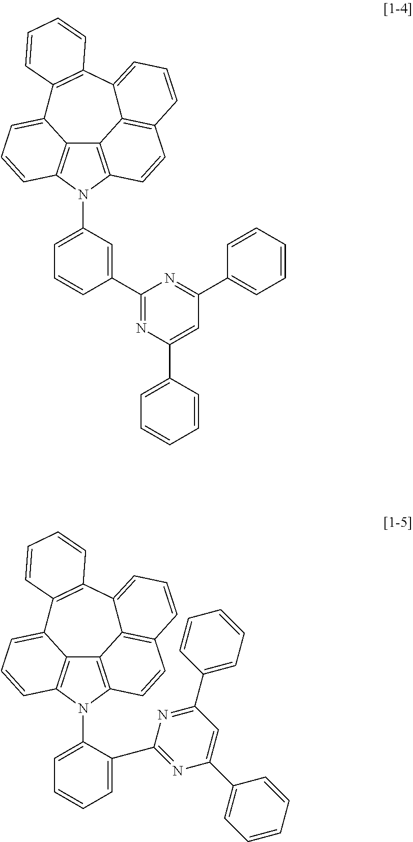 [SMILES: c1ccc(-c2cc(-c3ccccc3)nc(-c3cccc(-n4c5cccc6c5c5c7c(cccc7ccc54)-c4ccccc4-6)c3)n2)cc1 ; c1ccc(-c2cc(-c3ccccc3)nc(-c3ccccc3-n3c4cccc5c4c4c6c(cccc6ccc43)-c3ccccc3-5)n2)cc1]

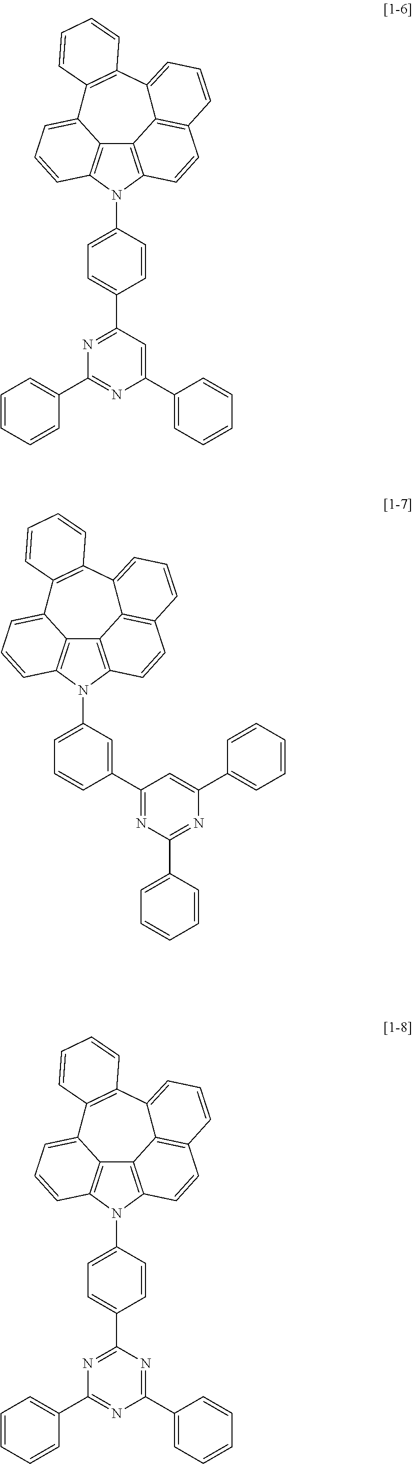 [SMILES: c1ccc(-c2cc(-c3ccc(-n4c5cccc6c5c5c7c(cccc7ccc54)-c4ccccc4-6)cc3)nc(-c3ccccc3)n2)cc1 ; c1ccc(-c2cc(-c3cccc(-n4c5cccc6c5c5c7c(cccc7ccc54)-c4ccccc4-6)c3)nc(-c3ccccc3)n2)cc1 ; c1ccc(-c2nc(-c3ccccc3)nc(-c3ccc(-n4c5cccc6c5c5c7c(cccc7ccc54)-c4ccccc4-6)cc3)n2)cc1]

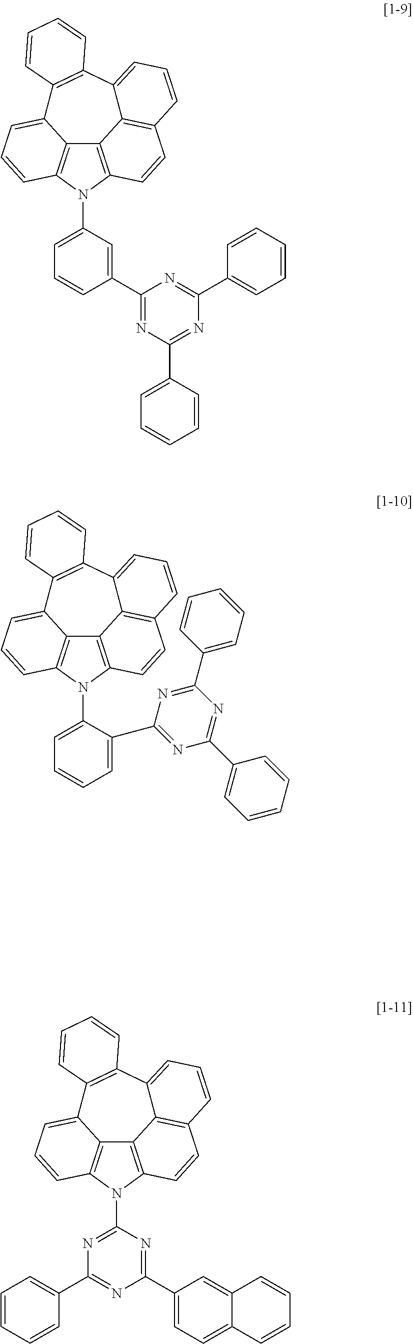 [SMILES: c1ccc(-c2nc(-c3ccccc3)nc(-c3cccc(-n4c5cccc6c5c5c7c(cccc7ccc54)-c4ccccc4-6)c3)n2)cc1 ; c1ccc(-c2nc(-c3ccccc3)nc(-c3ccccc3-n3c4cccc5c4c4c6c(cccc6ccc43)-c3ccccc3-5)n2)cc1 ; c1ccc(-c2nc(-c3ccc4ccccc4c3)nc(-n3c4cccc5c4c4c6c(cccc6ccc43)-c3ccccc3-5)n2)cc1]

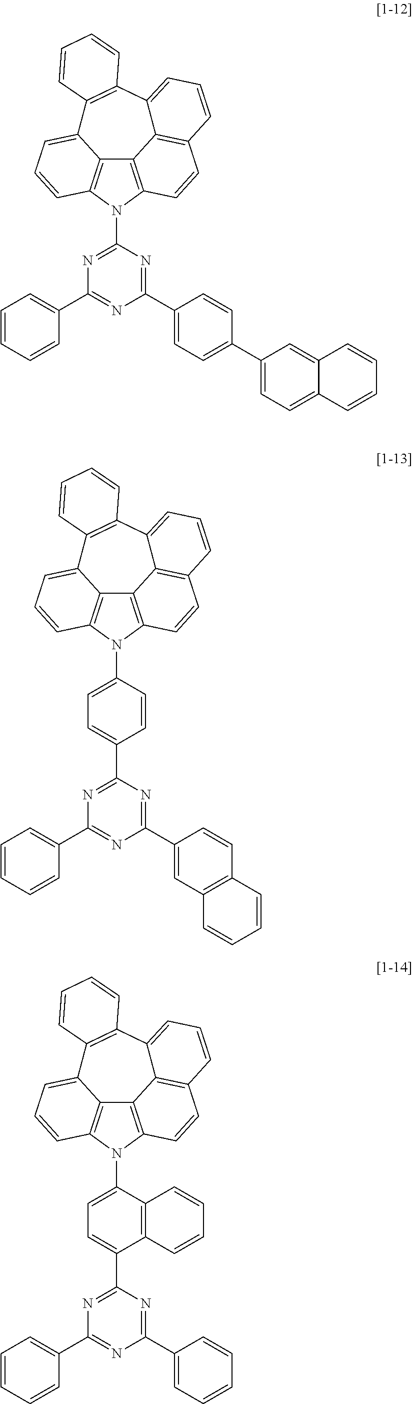 [SMILES: c1ccc(-c2nc(-c3ccc(-c4ccc5ccccc5c4)cc3)nc(-n3c4cccc5c4c4c6c(cccc6ccc43)-c3ccccc3-5)n2)cc1 ; c1ccc(-c2nc(-c3ccc(-n4c5cccc6c5c5c7c(cccc7ccc54)-c4ccccc4-6)cc3)nc(-c3ccc4ccccc4c3)n2)cc1 ; c1ccc(-c2nc(-c3ccccc3)nc(-c3ccc(-n4c5cccc6c5c5c7c(cccc7ccc54)-c4ccccc4-6)c4ccccc34)n2)cc1]

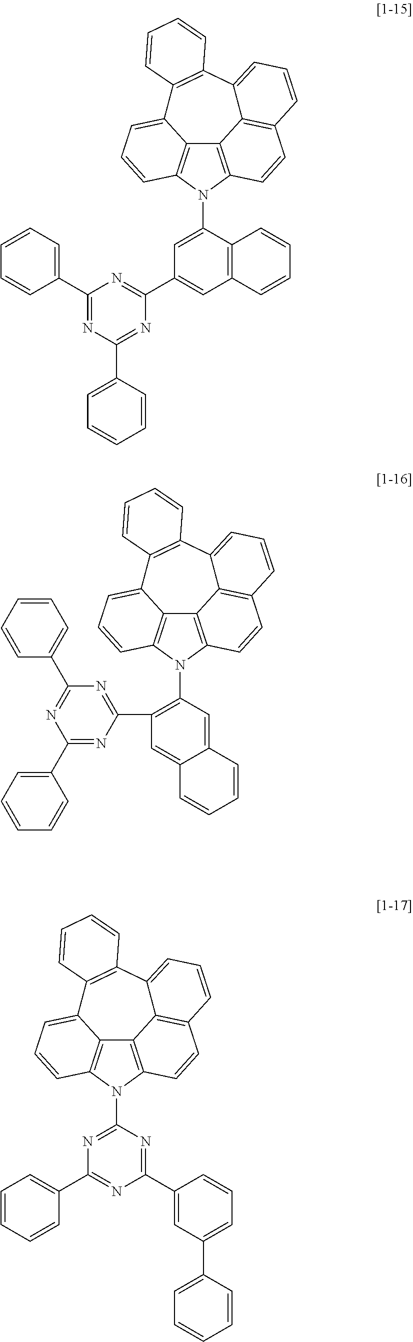 [SMILES: c1ccc(-c2nc(-c3ccccc3)nc(-c3cc(-n4c5cccc6c5c5c7c(cccc7ccc54)-c4ccccc4-6)c4ccccc4c3)n2)cc1 ; c1ccc(-c2nc(-c3ccccc3)nc(-c3cc4ccccc4cc3-n3c4cccc5c4c4c6c(cccc6ccc43)-c3ccccc3-5)n2)cc1 ; c1ccc(-c2cccc(-c3nc(-c4ccccc4)nc(-n4c5cccc6c5c5c7c(cccc7ccc54)-c4ccccc4-6)n3)c2)cc1]

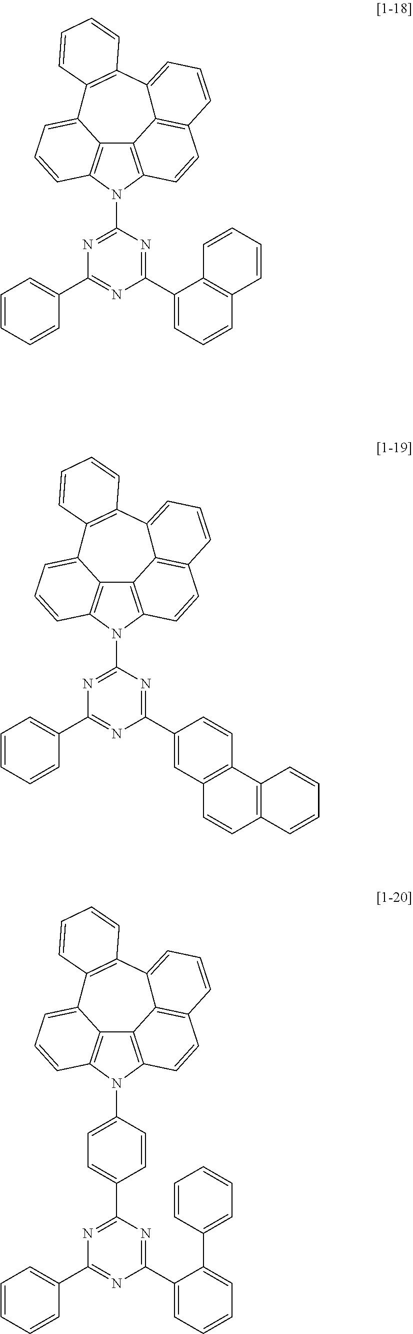

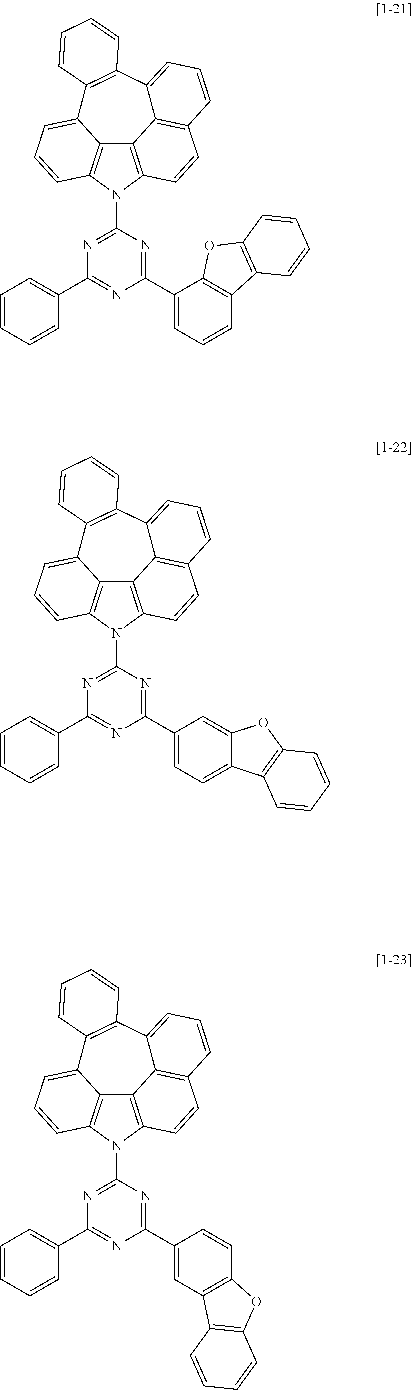 [SMILES: c1ccc(-c2nc(-c3cccc4c3oc3ccccc34)nc(-n3c4cccc5c4c4c6c(cccc6ccc43)-c3ccccc3-5)n2)cc1 ; c1ccc(-c2nc(-c3ccc4c(c3)oc3ccccc34)nc(-n3c4cccc5c4c4c6c(cccc6ccc43)-c3ccccc3-5)n2)cc1 ; c1ccc(-c2nc(-c3ccc4oc5ccccc5c4c3)nc(-n3c4cccc5c4c4c6c(cccc6ccc43)-c3ccccc3-5)n2)cc1]

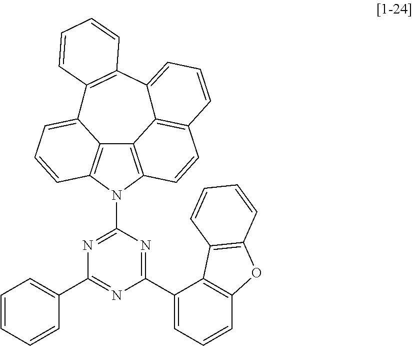 [SMILES: c1ccc(-c2nc(-c3cccc4oc5ccccc5c34)nc(-n3c4cccc5c4c4c6c(cccc6ccc43)-c3ccccc3-5)n2)cc1]

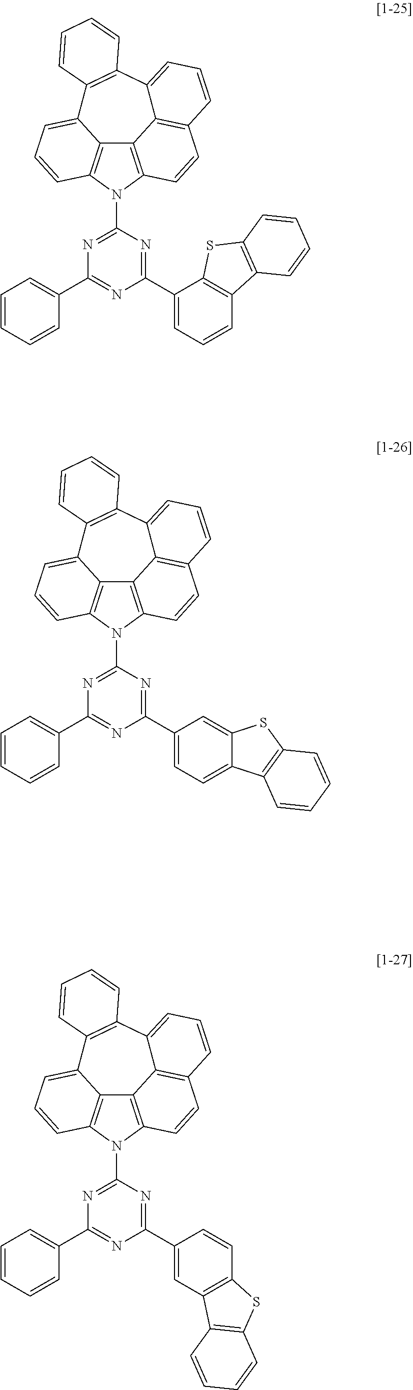 [SMILES: c1ccc(-c2nc(-c3cccc4c3sc3ccccc34)nc(-n3c4cccc5c4c4c6c(cccc6ccc43)-c3ccccc3-5)n2)cc1 ; c1ccc(-c2nc(-c3ccc4c(c3)sc3ccccc34)nc(-n3c4cccc5c4c4c6c(cccc6ccc43)-c3ccccc3-5)n2)cc1 ; c1ccc(-c2nc(-c3ccc4sc5ccccc5c4c3)nc(-n3c4cccc5c4c4c6c(cccc6ccc43)-c3ccccc3-5)n2)cc1]

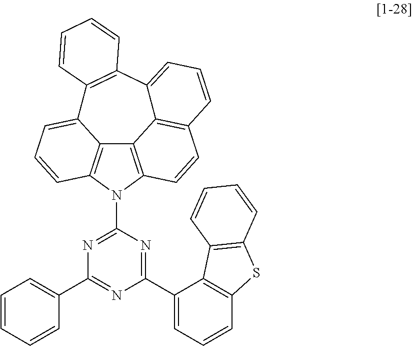 [SMILES: c1ccc(-c2nc(-c3cccc4sc5ccccc5c34)nc(-n3c4cccc5c4c4c6c(cccc6ccc43)-c3ccccc3-5)n2)cc1]

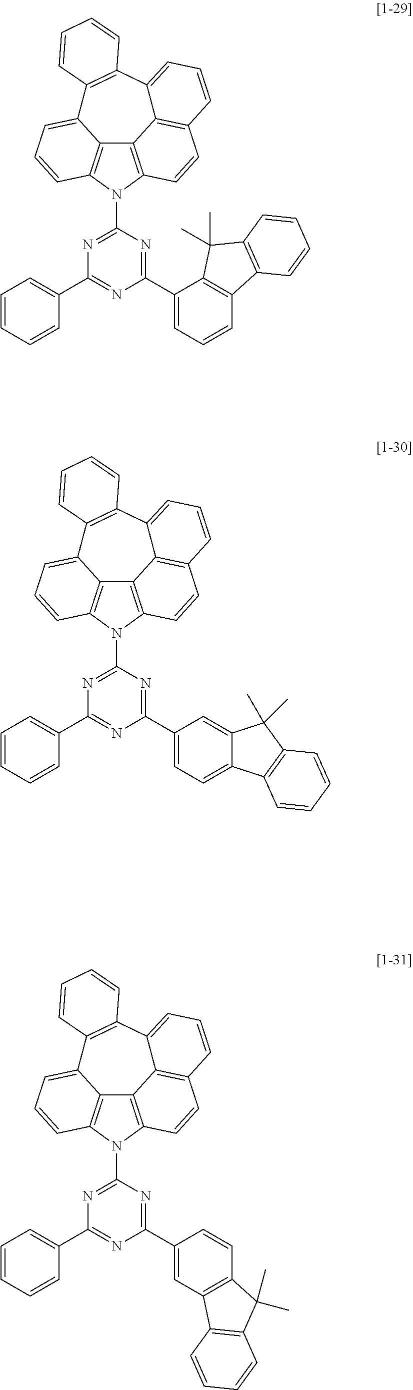 [SMILES: CC1(C)c2ccccc2-c2cccc(-c3nc(-c4ccccc4)nc(-n4c5cccc6c5c5c7c(cccc7ccc54)-c4ccccc4-6)n3)c21 ; CC1(C)c2ccccc2-c2ccc(-c3nc(-c4ccccc4)nc(-n4c5cccc6c5c5c7c(cccc7ccc54)-c4ccccc4-6)n3)cc21 ; CC1(C)c2ccccc2-c2cc(-c3nc(-c4ccccc4)nc(-n4c5cccc6c5c5c7c(cccc7ccc54)-c4ccccc4-6)n3)ccc21]

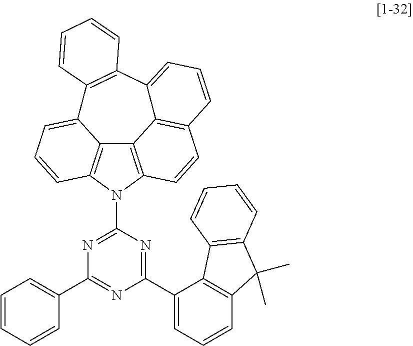 [SMILES: CC1(C)c2ccccc2-c2c(-c3nc(-c4ccccc4)nc(-n4c5cccc6c5c5c7c(cccc7ccc54)-c4ccccc4-6)n3)cccc21]

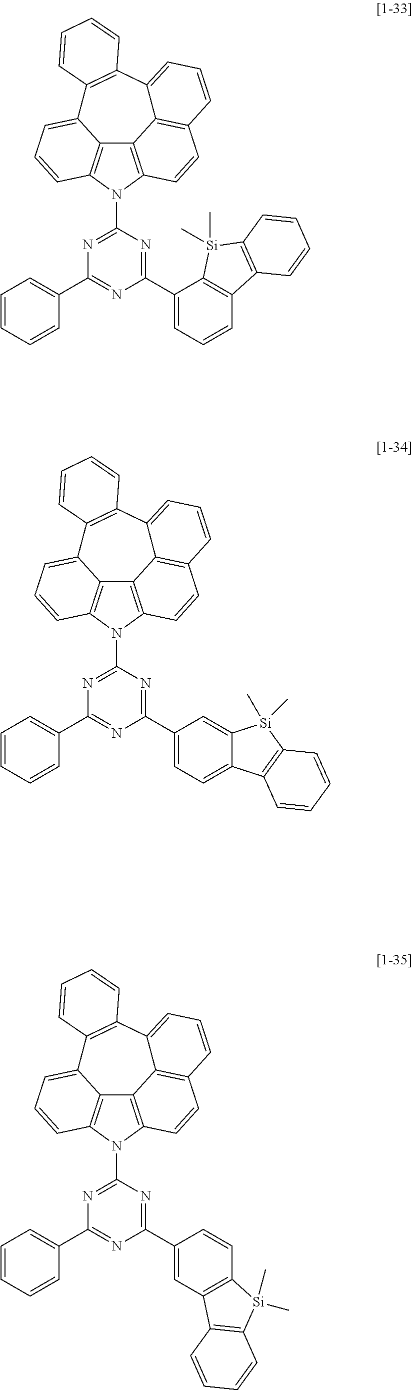 [SMILES: C[Si]1(C)c2ccccc2-c2cccc(-c3nc(-c4ccccc4)nc(-n4c5cccc6c5c5c7c(cccc7ccc54)-c4ccccc4-6)n3)c21 ; C[Si]1(C)c2ccccc2-c2ccc(-c3nc(-c4ccccc4)nc(-n4c5cccc6c5c5c7c(cccc7ccc54)-c4ccccc4-6)n3)cc21 ; C[Si]1(C)c2ccccc2-c2cc(-c3nc(-c4ccccc4)nc(-n4c5cccc6c5c5c7c(cccc7ccc54)-c4ccccc4-6)n3)ccc21]

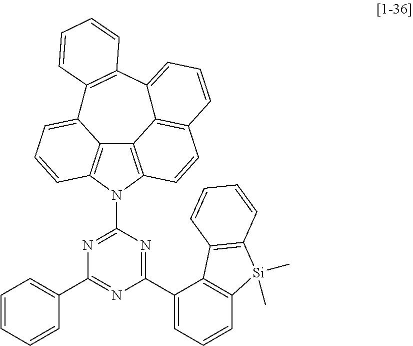 [SMILES: C[Si]1(C)c2ccccc2-c2c(-c3nc(-c4ccccc4)nc(-n4c5cccc6c5c5c7c(cccc7ccc54)-c4ccccc4-6)n3)cccc21]

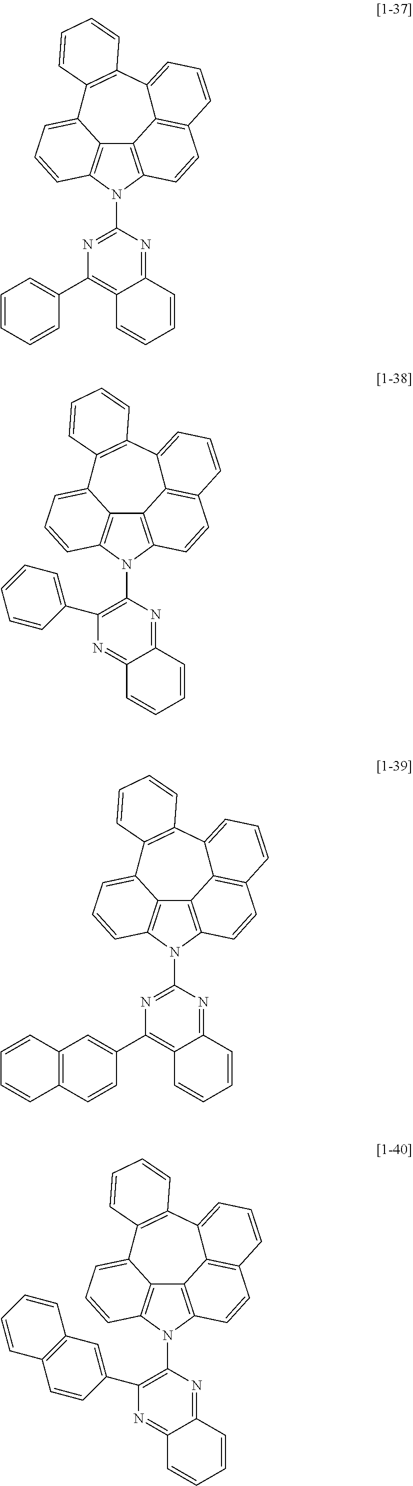 [SMILES: c1ccc(-c2nc(-n3c4cccc5c4c4c6c(cccc6ccc43)-c3ccccc3-5)nc3ccccc23)cc1 ; c1ccc(-c2nc3ccccc3nc2-n2c3cccc4c3c3c5c(cccc5ccc32)-c2ccccc2-4)cc1 ; c1ccc2c(c1)-c1cccc3ccc4c(c13)c1c-2cccc1n4-c1nc(-c2ccc3ccccc3c2)c2ccccc2n1 ; c1ccc2c(c1)-c1cccc3ccc4c(c13)c1c-2cccc1n4-c1nc2ccccc2nc1-c1ccc2ccccc2c1]

In an implementation, the second compound may be, e.g., represented by one of Chemical Formula 2A to Chemical Formula 2J according to the type and fusion direction of the A ring.

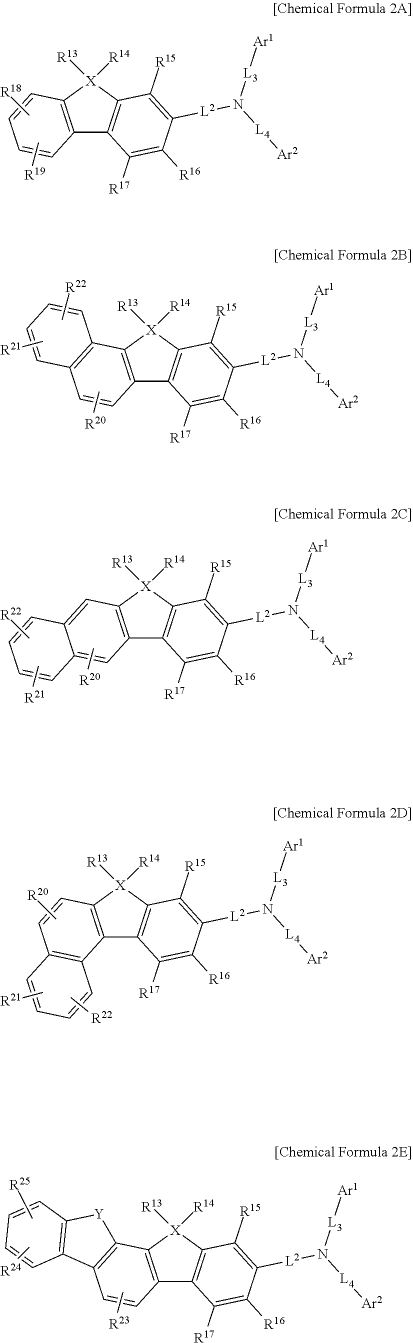

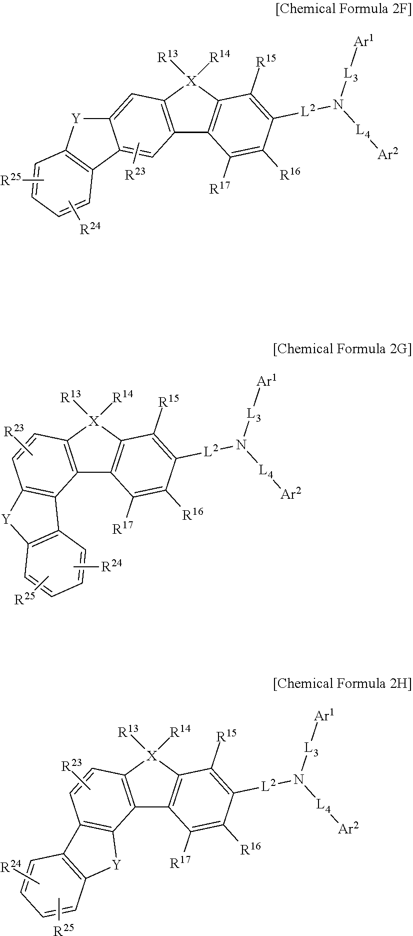

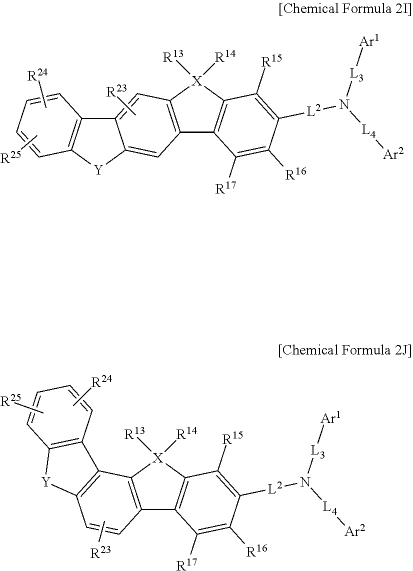

In Chemical Formula 2A to Chemical Formula 2J, X, Y, R¹³ to R²⁵, L² to L⁴, Ar¹, and Ar² may be defined the same as those described above.

In an implementation, R¹³ and R¹⁴ may each independently be, e.g., a substituted or unsubstituted C1 to C10 alkyl group, or a substituted or unsubstituted C6 to C12 aryl group.

In an implementation, R¹³ and R¹⁴ may each independently be, e.g., a substituted or unsubstituted methyl group, a substituted or unsubstituted ethyl group, a substituted or unsubstituted phenyl group, or a substituted or unsubstituted biphenyl group.

In an implementation, R¹⁵ to R²⁵ may each independently be, e.g., hydrogen, deuterium, a substituted or unsubstituted C1 to C10 alkyl group, or a substituted or unsubstituted C6 to C12 aryl group.

In an implementation, R¹⁵ to R²⁵ may each independently be, e.g., hydrogen, deuterium, a substituted or unsubstituted C1 to C5 alkyl group, a substituted or unsubstituted phenyl group, or a substituted or unsubstituted biphenyl group.

In an implementation, L² may be, e.g., a single bond or a substituted or unsubstituted phenylene group.

In an implementation, L² may be a single bond.

In an implementation, L³ and L⁴ may each independently be, e.g., a single bond, a substituted or unsubstituted phenylene group, a substituted or unsubstituted biphenylene group, a substituted or unsubstituted naphthylene group, or a substituted or unsubstituted fluorenylene group.

In an implementation, L³ and L⁴ may each independently be, e.g., a single bond, a substituted or unsubstituted phenylene group, or a substituted or unsubstituted biphenylene group.

In an implementation, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted anthracenyl group, a substituted or unsubstituted phenanthrenyl group, a substituted or unsubstituted triphenylene group, a substituted or unsubstituted dibenzofuranyl group, or a substituted or unsubstituted dibenzothiophenyl group.

In an implementation, Ar¹ and Ar² may each independently be, e.g., a substituted or unsubstituted phenyl group, a substituted or unsubstituted biphenyl group, or a substituted or unsubstituted naphthyl group.

In an implementation, moieties *-L³-Ar¹ and *-L⁴-Ar² may each independently be, e.g., a moiety of Group III.

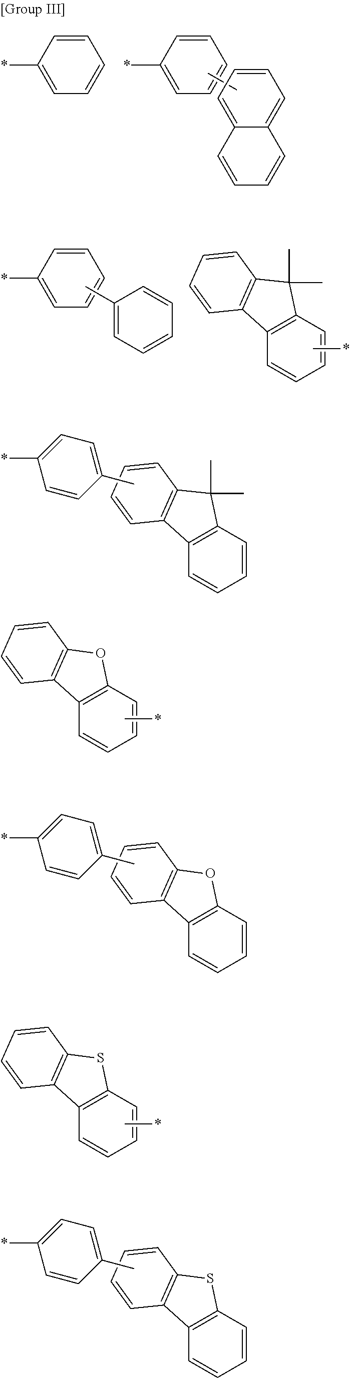

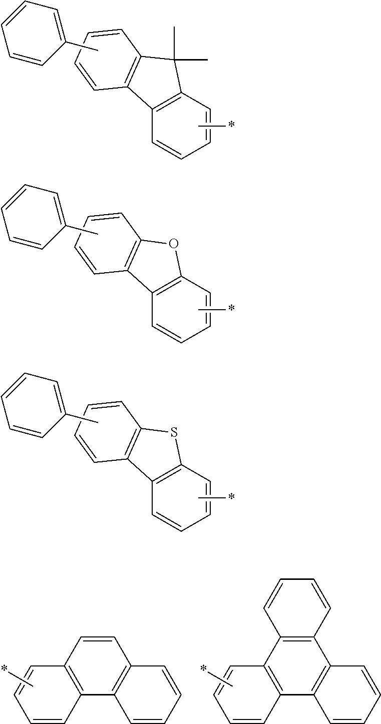

In Group III, * is a linking point (e.g., to N of Chemical Formula 2).

In an implementation, Chemical Formula 2A may be, e.g., represented by Chemical Formula 2A-1.

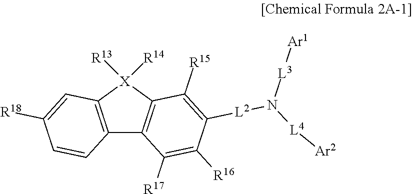

In Chemical Formula 2A-1, X, L² to L⁴, R¹³ to R¹⁷, Ar¹, and Ar² may be defined the same as those described above.

R¹⁸ may be, e.g., a substituted or unsubstituted phenyl group or a substituted or unsubstituted biphenyl group.

In an implementation, the second compound may be represented by, e.g., Chemical Formula 2E, Chemical Formula 2F, Chemical Formula 2G, Chemical Formula 2H, Chemical Formula 2I, or Chemical Formula 2J.

In an implementation, the second compound may be represented by, e.g., Chemical Formula 2H or Chemical Formula 2J.

In an implementation, the second compound may be represented by, e.g., Chemical Formula 2H.

In an implementation, the second compound may be, e.g., a compound of Group 2.

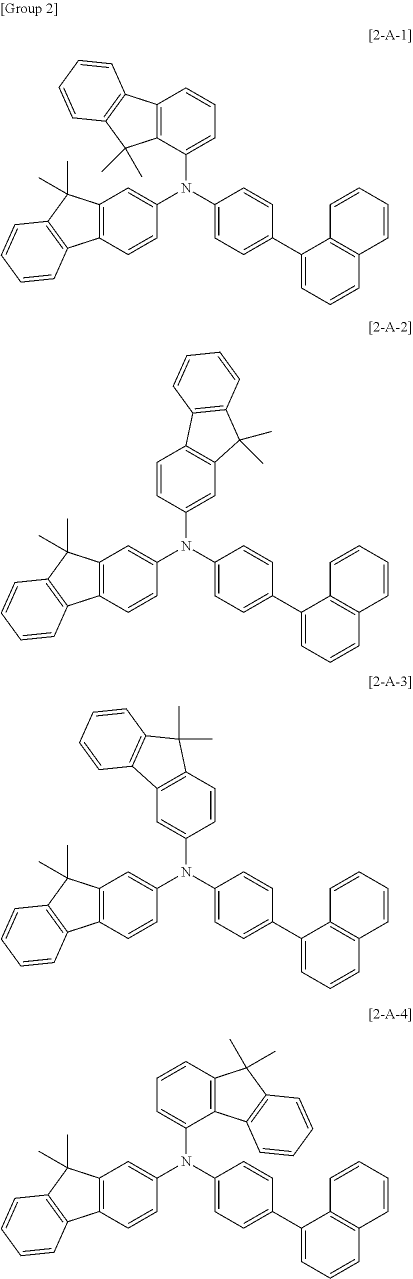 [SMILES: CC1(C)c2ccccc2-c2ccc(N(c3ccc(-c4cccc5ccccc45)cc3)c3cccc4c3C(C)(C)c3ccccc3-4)cc21 ; CC1(C)c2ccccc2-c2ccc(N(c3ccc(-c4cccc5ccccc45)cc3)c3ccc4c(c3)C(C)(C)c3ccccc3-4)cc21 ; CC1(C)c2ccccc2-c2cc(N(c3ccc(-c4cccc5ccccc45)cc3)c3ccc4c(c3)C(C)(C)c3ccccc3-4)ccc21 ; CC1(C)c2ccccc2-c2ccc(N(c3ccc(-c4cccc5ccccc45)cc3)c3cccc4c3-c3ccccc3C4(C)C)cc21]

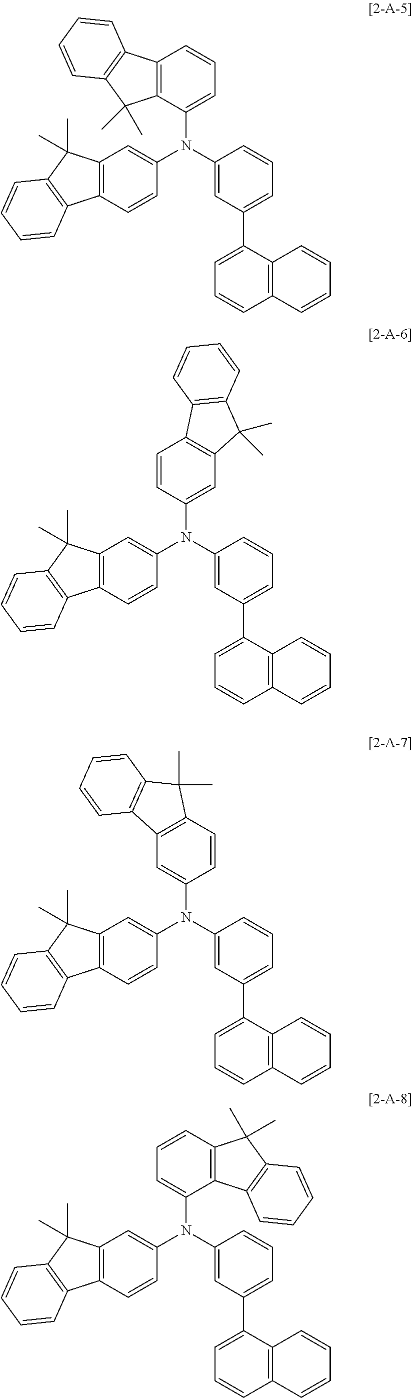

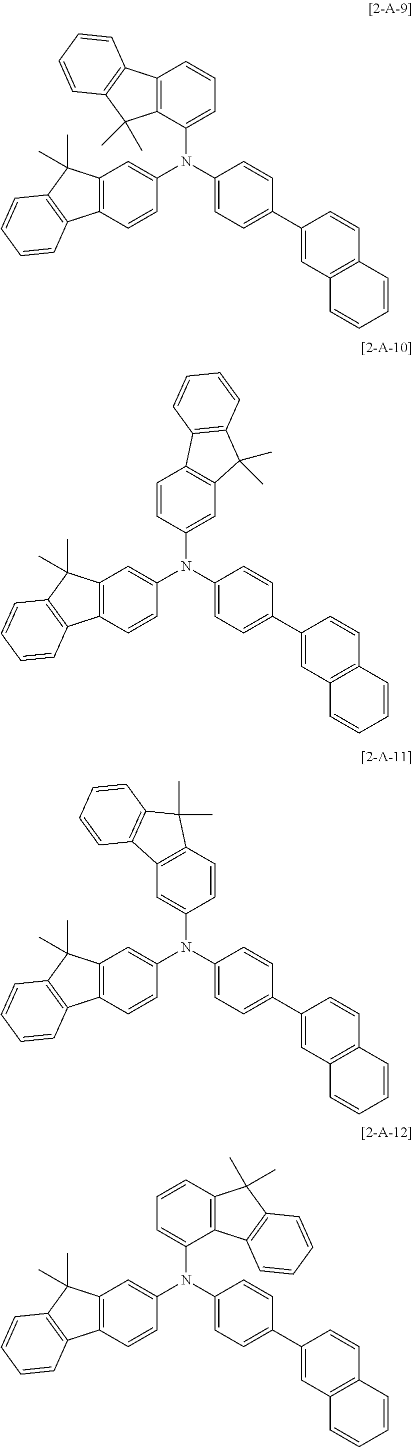 [SMILES: CC1(C)c2ccccc2-c2ccc(N(c3ccc(-c4ccc5ccccc5c4)cc3)c3cccc4c3C(C)(C)c3ccccc3-4)cc21 ; CC1(C)c2ccccc2-c2ccc(N(c3ccc(-c4ccc5ccccc5c4)cc3)c3ccc4c(c3)C(C)(C)c3ccccc3-4)cc21 ; CC1(C)c2ccccc2-c2cc(N(c3ccc(-c4ccc5ccccc5c4)cc3)c3ccc4c(c3)C(C)(C)c3ccccc3-4)ccc21 ; CC1(C)c2ccccc2-c2ccc(N(c3ccc(-c4ccc5ccccc5c4)cc3)c3cccc4c3-c3ccccc3C4(C)C)cc21]

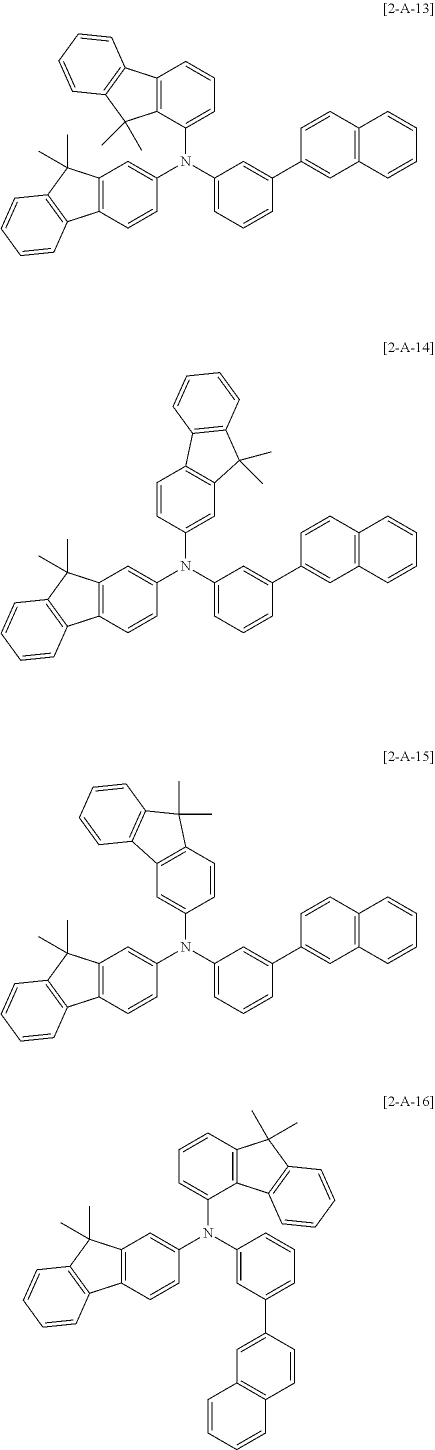 [SMILES: CC1(C)c2ccccc2-c2ccc(N(c3cccc(-c4ccc5ccccc5c4)c3)c3cccc4c3C(C)(C)c3ccccc3-4)cc21 ; CC1(C)c2ccccc2-c2ccc(N(c3cccc(-c4ccc5ccccc5c4)c3)c3ccc4c(c3)C(C)(C)c3ccccc3-4)cc21 ; CC1(C)c2ccccc2-c2cc(N(c3cccc(-c4ccc5ccccc5c4)c3)c3ccc4c(c3)C(C)(C)c3ccccc3-4)ccc21 ; CC1(C)c2ccccc2-c2ccc(N(c3cccc(-c4ccc5ccccc5c4)c3)c3cccc4c3-c3ccccc3C4(C)C)cc21]

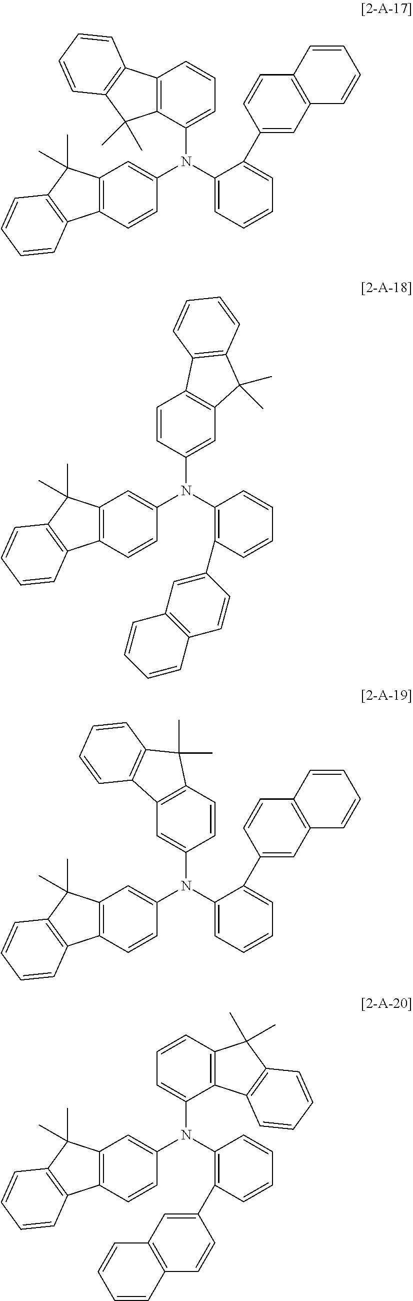 [SMILES: CC1(C)c2ccccc2-c2ccc(N(c3ccccc3-c3ccc4ccccc4c3)c3cccc4c3C(C)(C)c3ccccc3-4)cc21 ; CC1(C)c2ccccc2-c2ccc(N(c3ccc4c(c3)C(C)(C)c3ccccc3-4)c3ccccc3-c3ccc4ccccc4c3)cc21 ; CC1(C)c2ccccc2-c2cc(N(c3ccc4c(c3)C(C)(C)c3ccccc3-4)c3ccccc3-c3ccc4ccccc4c3)ccc21 ; CC1(C)c2ccccc2-c2ccc(N(c3ccccc3-c3ccc4ccccc4c3)c3cccc4c3-c3ccccc3C4(C)C)cc21]

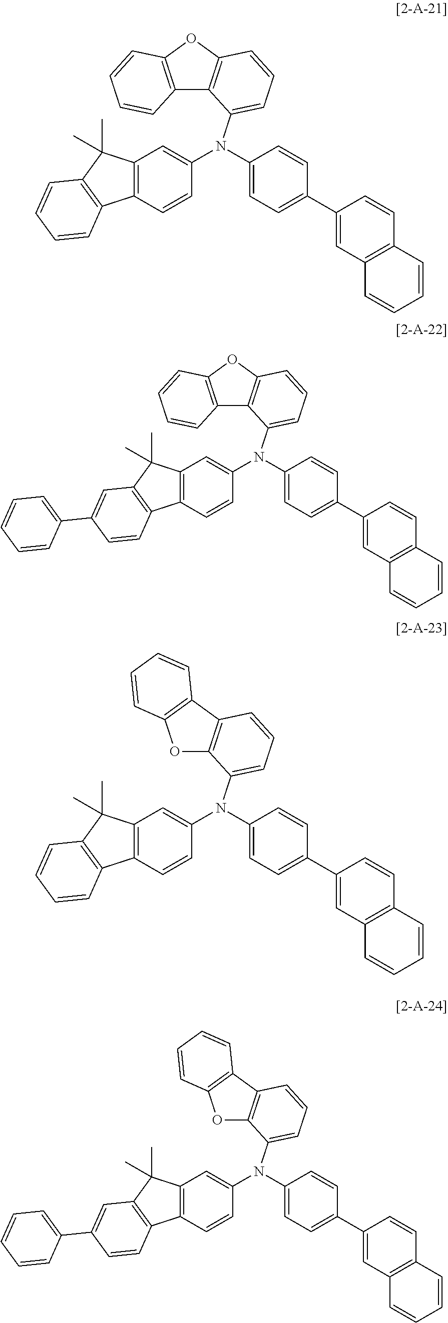 [SMILES: CC1(C)c2ccccc2-c2ccc(N(c3ccc(-c4ccc5ccccc5c4)cc3)c3cccc4oc5ccccc5c34)cc21 ; CC1(C)c2cc(-c3ccccc3)ccc2-c2ccc(N(c3ccc(-c4ccc5ccccc5c4)cc3)c3cccc4oc5ccccc5c34)cc21 ; CC1(C)c2ccccc2-c2ccc(N(c3ccc(-c4ccc5ccccc5c4)cc3)c3cccc4c3oc3ccccc34)cc21 ; CC1(C)c2cc(-c3ccccc3)ccc2-c2ccc(N(c3ccc(-c4ccc5ccccc5c4)cc3)c3cccc4c3oc3ccccc34)cc21]

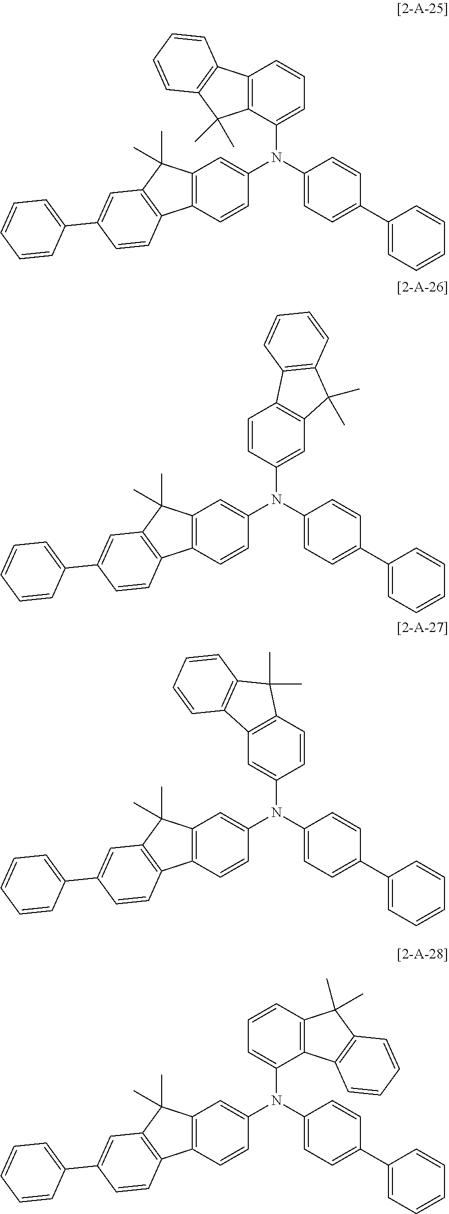 [SMILES: CC1(C)c2cc(-c3ccccc3)ccc2-c2ccc(N(c3ccc(-c4ccccc4)cc3)c3cccc4c3C(C)(C)c3ccccc3-4)cc21 ; CC1(C)c2ccccc2-c2ccc(N(c3ccc(-c4ccccc4)cc3)c3ccc4c(c3)C(C)(C)c3cc(-c5ccccc5)ccc3-4)cc21 ; CC1(C)c2ccccc2-c2cc(N(c3ccc(-c4ccccc4)cc3)c3ccc4c(c3)C(C)(C)c3cc(-c5ccccc5)ccc3-4)ccc21 ; CC1(C)c2cc(-c3ccccc3)ccc2-c2ccc(N(c3ccc(-c4ccccc4)cc3)c3cccc4c3-c3ccccc3C4(C)C)cc21]

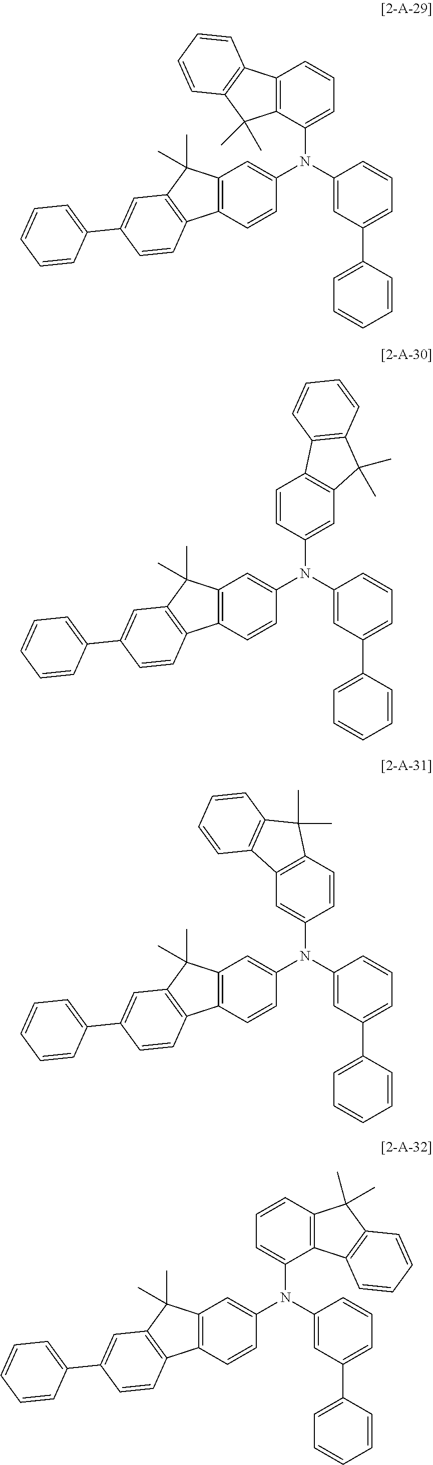 [SMILES: CC1(C)c2cc(-c3ccccc3)ccc2-c2ccc(N(c3cccc(-c4ccccc4)c3)c3cccc4c3C(C)(C)c3ccccc3-4)cc21 ; CC1(C)c2ccccc2-c2ccc(N(c3cccc(-c4ccccc4)c3)c3ccc4c(c3)C(C)(C)c3cc(-c5ccccc5)ccc3-4)cc21 ; CC1(C)c2ccccc2-c2cc(N(c3cccc(-c4ccccc4)c3)c3ccc4c(c3)C(C)(C)c3cc(-c5ccccc5)ccc3-4)ccc21 ; CC1(C)c2cc(-c3ccccc3)ccc2-c2ccc(N(c3cccc(-c4ccccc4)c3)c3cccc4c3-c3ccccc3C4(C)C)cc21]

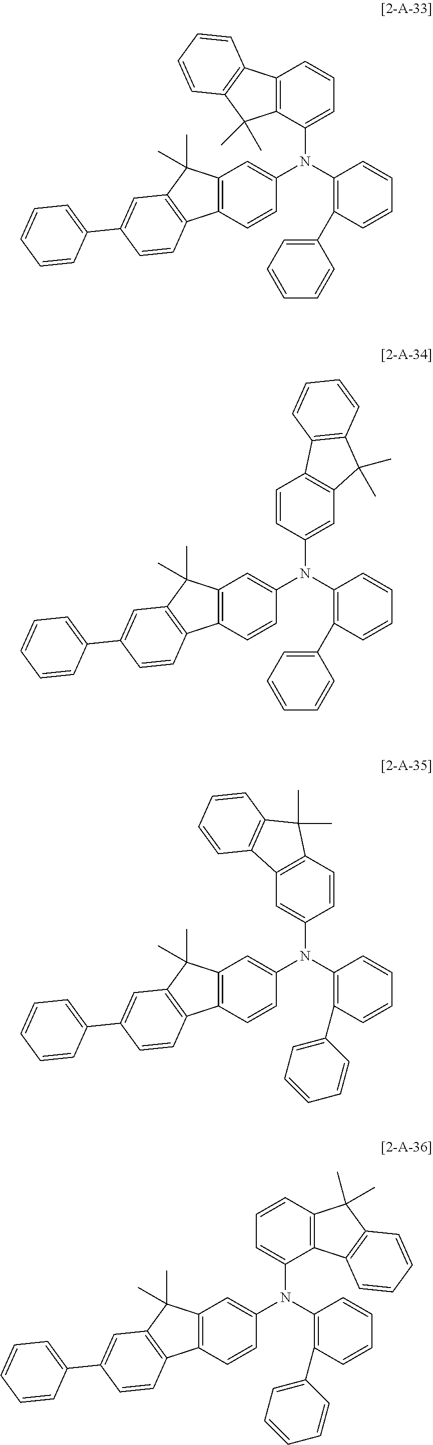 [SMILES: CC1(C)c2cc(-c3ccccc3)ccc2-c2ccc(N(c3ccccc3-c3ccccc3)c3cccc4c3C(C)(C)c3ccccc3-4)cc21 ; CC1(C)c2ccccc2-c2ccc(N(c3ccc4c(c3)C(C)(C)c3cc(-c5ccccc5)ccc3-4)c3ccccc3-c3ccccc3)cc21 ; CC1(C)c2ccccc2-c2cc(N(c3ccc4c(c3)C(C)(C)c3cc(-c5ccccc5)ccc3-4)c3ccccc3-c3ccccc3)ccc21 ; CC1(C)c2cc(-c3ccccc3)ccc2-c2ccc(N(c3ccccc3-c3ccccc3)c3cccc4c3-c3ccccc3C4(C)C)cc21]

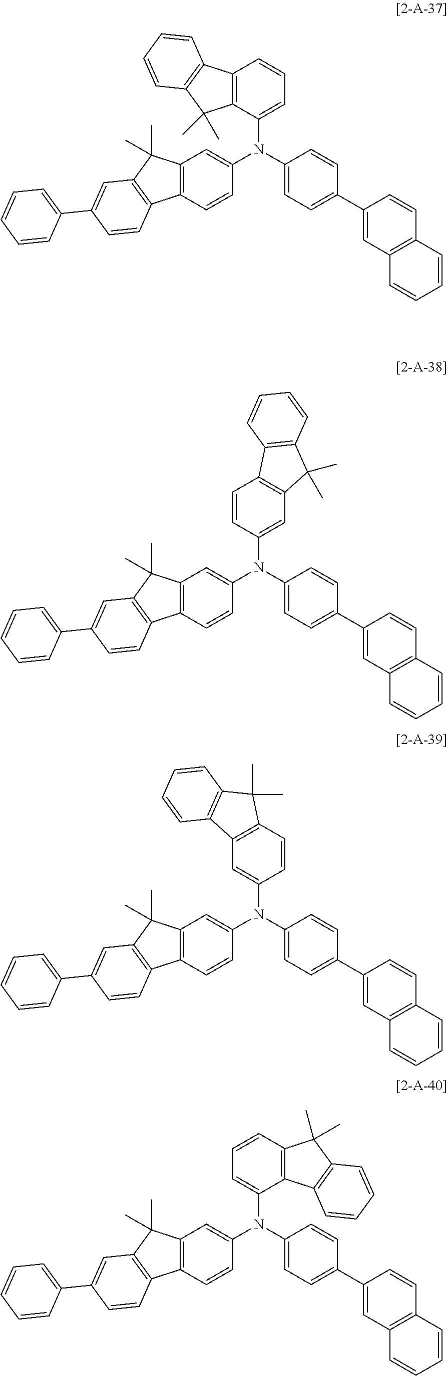 [SMILES: CC1(C)c2cc(-c3ccccc3)ccc2-c2ccc(N(c3ccc(-c4ccc5ccccc5c4)cc3)c3cccc4c3C(C)(C)c3ccccc3-4)cc21 ; CC1(C)c2ccccc2-c2ccc(N(c3ccc(-c4ccc5ccccc5c4)cc3)c3ccc4c(c3)C(C)(C)c3cc(-c5ccccc5)ccc3-4)cc21 ; CC1(C)c2ccccc2-c2cc(N(c3ccc(-c4ccc5ccccc5c4)cc3)c3ccc4c(c3)C(C)(C)c3cc(-c5ccccc5)ccc3-4)ccc21 ; CC1(C)c2cc(-c3ccccc3)ccc2-c2ccc(N(c3ccc(-c4ccc5ccccc5c4)cc3)c3cccc4c3-c3ccccc3C4(C)C)cc21]

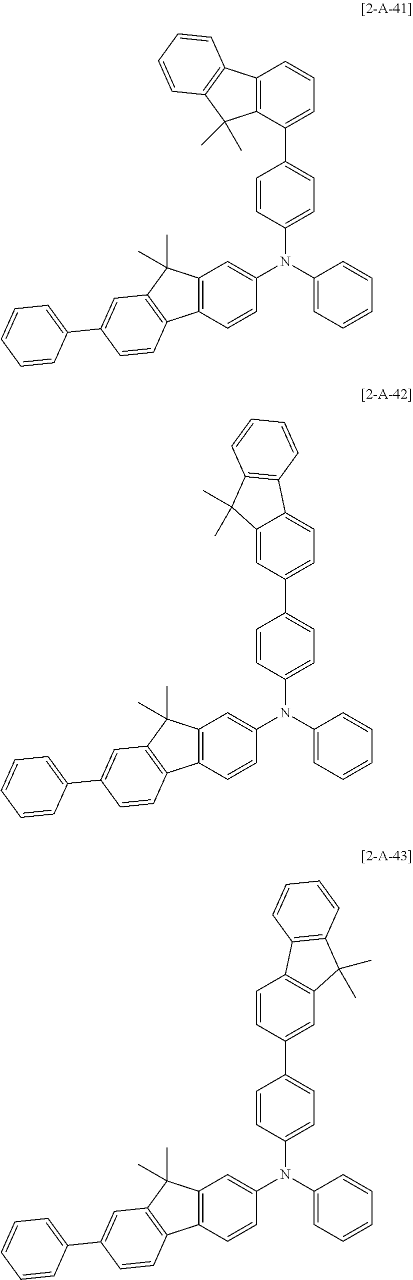 [SMILES: CC1(C)c2cc(-c3ccccc3)ccc2-c2ccc(N(c3ccccc3)c3ccc(-c4cccc5c4C(C)(C)c4ccccc4-5)cc3)cc21 ; CC1(C)c2ccccc2-c2ccc(-c3ccc(N(c4ccccc4)c4ccc5c(c4)C(C)(C)c4cc(-c6ccccc6)ccc4-5)cc3)cc21 ; CC1(C)c2ccccc2-c2ccc(-c3ccc(N(c4ccccc4)c4ccc5c(c4)C(C)(C)c4cc(-c6ccccc6)ccc4-5)cc3)cc21]

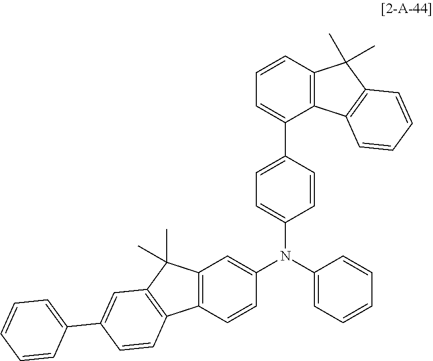 [SMILES: CC1(C)c2cc(-c3ccccc3)ccc2-c2ccc(N(c3ccccc3)c3ccc(-c4cccc5c4-c4ccccc4C5(C)C)cc3)cc21]

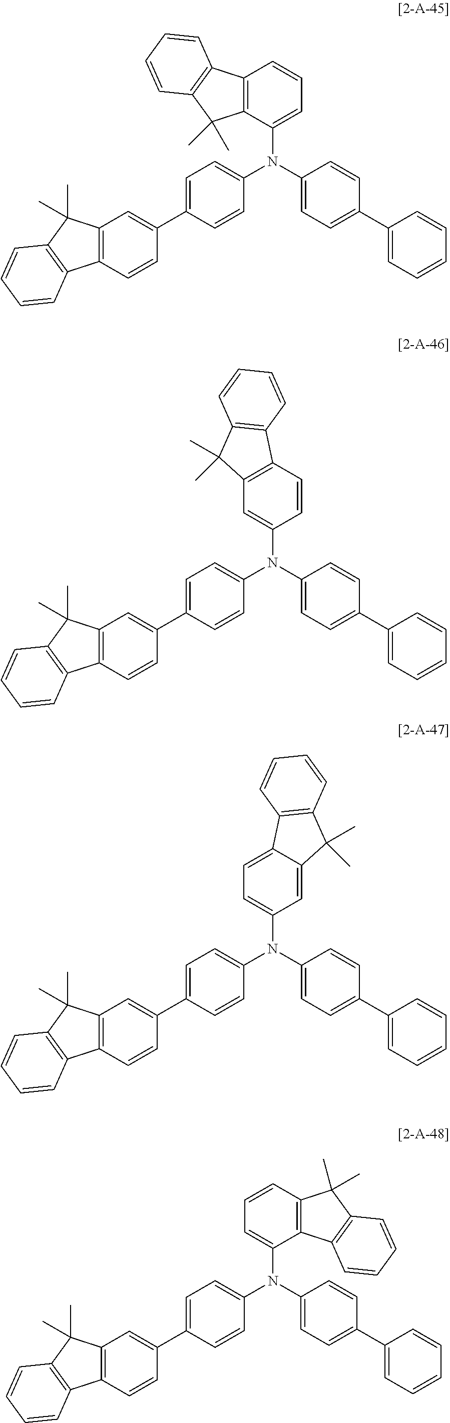 [SMILES: CC1(C)c2ccccc2-c2ccc(-c3ccc(N(c4ccc(-c5ccccc5)cc4)c4cccc5c4C(C)(C)c4ccccc4-5)cc3)cc21 ; CC1(C)c2ccccc2-c2ccc(-c3ccc(N(c4ccc(-c5ccccc5)cc4)c4ccc5c(c4)C(C)(C)c4ccccc4-5)cc3)cc21 ; CC1(C)c2ccccc2-c2ccc(-c3ccc(N(c4ccc(-c5ccccc5)cc4)c4ccc5c(c4)C(C)(C)c4ccccc4-5)cc3)cc21 ; CC1(C)c2ccccc2-c2ccc(-c3ccc(N(c4ccc(-c5ccccc5)cc4)c4cccc5c4-c4ccccc4C5(C)C)cc3)cc21]

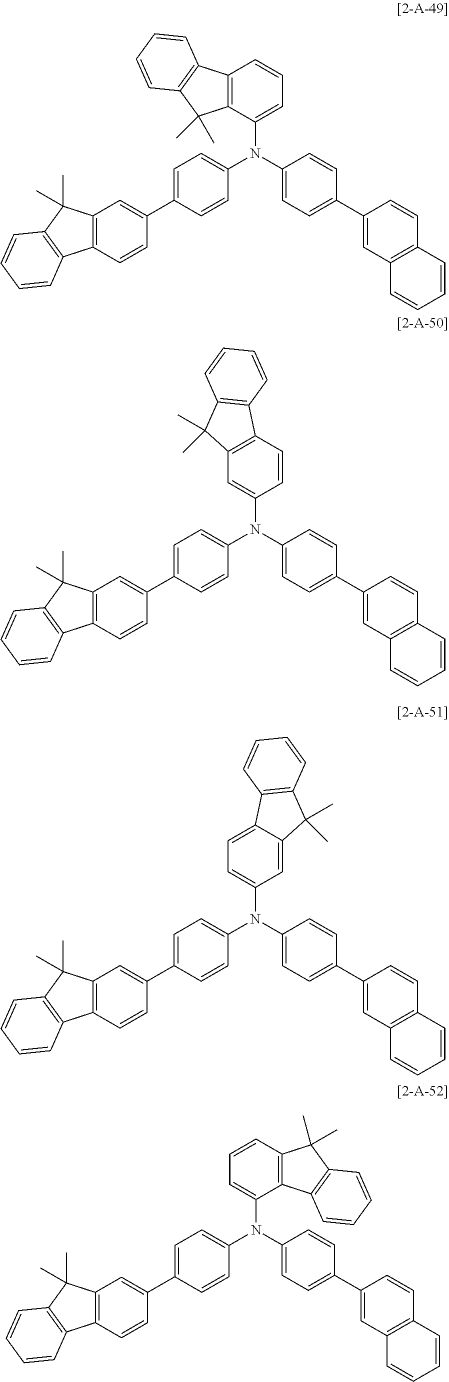 [SMILES: CC1(C)c2ccccc2-c2ccc(-c3ccc(N(c4ccc(-c5ccc6ccccc6c5)cc4)c4cccc5c4C(C)(C)c4ccccc4-5)cc3)cc21 ; CC1(C)c2ccccc2-c2ccc(-c3ccc(N(c4ccc(-c5ccc6ccccc6c5)cc4)c4ccc5c(c4)C(C)(C)c4ccccc4-5)cc3)cc21 ; CC1(C)c2ccccc2-c2ccc(-c3ccc(N(c4ccc(-c5ccc6ccccc6c5)cc4)c4ccc5c(c4)C(C)(C)c4ccccc4-5)cc3)cc21 ; CC1(C)c2ccccc2-c2ccc(-c3ccc(N(c4ccc(-c5ccc6ccccc6c5)cc4)c4cccc5c4-c4ccccc4C5(C)C)cc3)cc21]

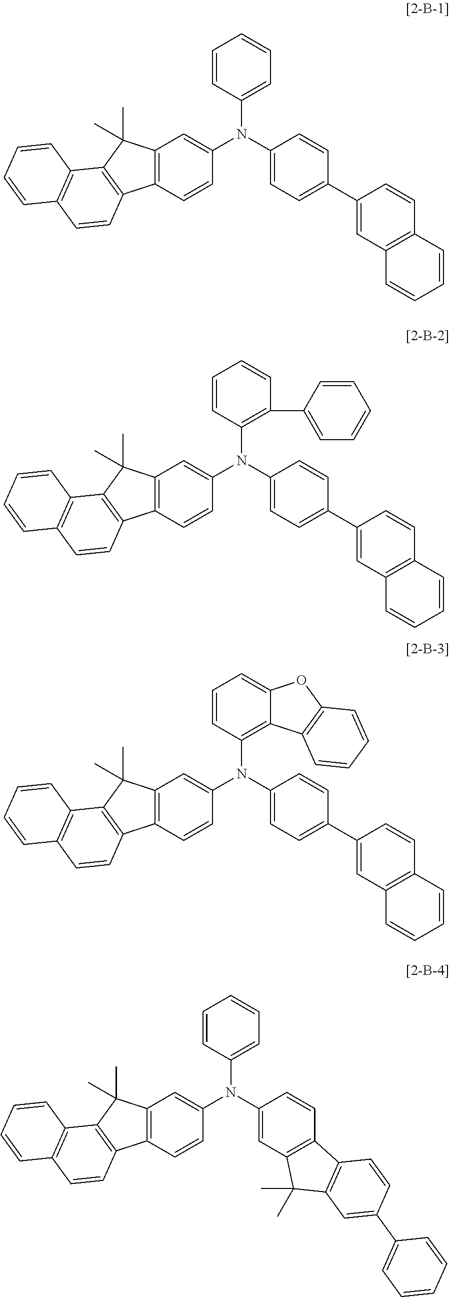 [SMILES: CC1(C)c2cc(N(c3ccccc3)c3ccc(-c4ccc5ccccc5c4)cc3)ccc2-c2ccc3ccccc3c21 ; CC1(C)c2cc(N(c3ccc(-c4ccc5ccccc5c4)cc3)c3ccccc3-c3ccccc3)ccc2-c2ccc3ccccc3c21 ; CC1(C)c2cc(N(c3ccc(-c4ccc5ccccc5c4)cc3)c3cccc4oc5ccccc5c34)ccc2-c2ccc3ccccc3c21 ; CC1(C)c2cc(-c3ccccc3)ccc2-c2ccc(N(c3ccccc3)c3ccc4c(c3)C(C)(C)c3c-4ccc4ccccc34)cc21]

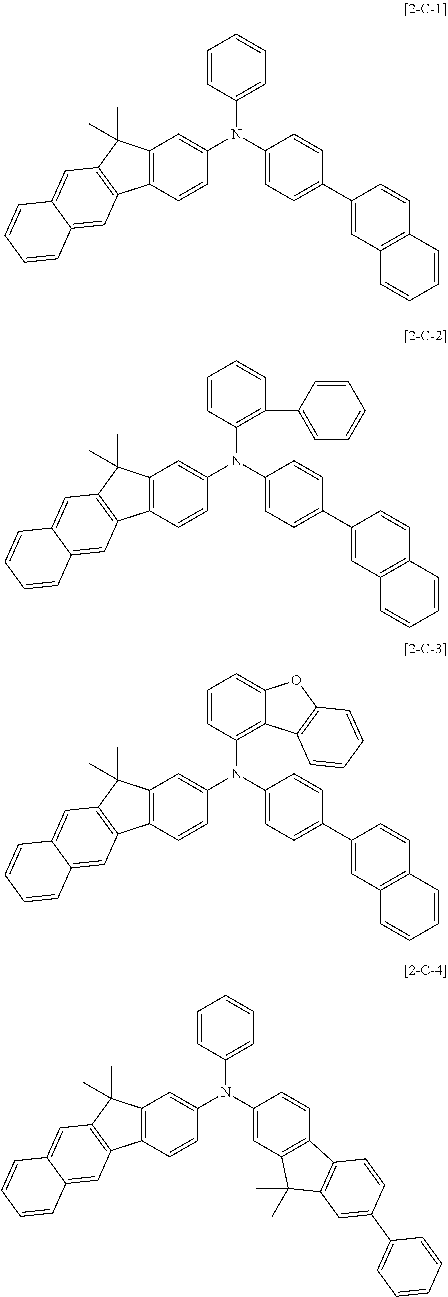 [SMILES: CC1(C)c2cc(N(c3ccccc3)c3ccc(-c4ccc5ccccc5c4)cc3)ccc2-c2cc3ccccc3cc21 ; CC1(C)c2cc(N(c3ccc(-c4ccc5ccccc5c4)cc3)c3ccccc3-c3ccccc3)ccc2-c2cc3ccccc3cc21 ; CC1(C)c2cc(N(c3ccc(-c4ccc5ccccc5c4)cc3)c3cccc4oc5ccccc5c34)ccc2-c2cc3ccccc3cc21 ; CC1(C)c2cc(-c3ccccc3)ccc2-c2ccc(N(c3ccccc3)c3ccc4c(c3)C(C)(C)c3cc5ccccc5cc3-4)cc21]

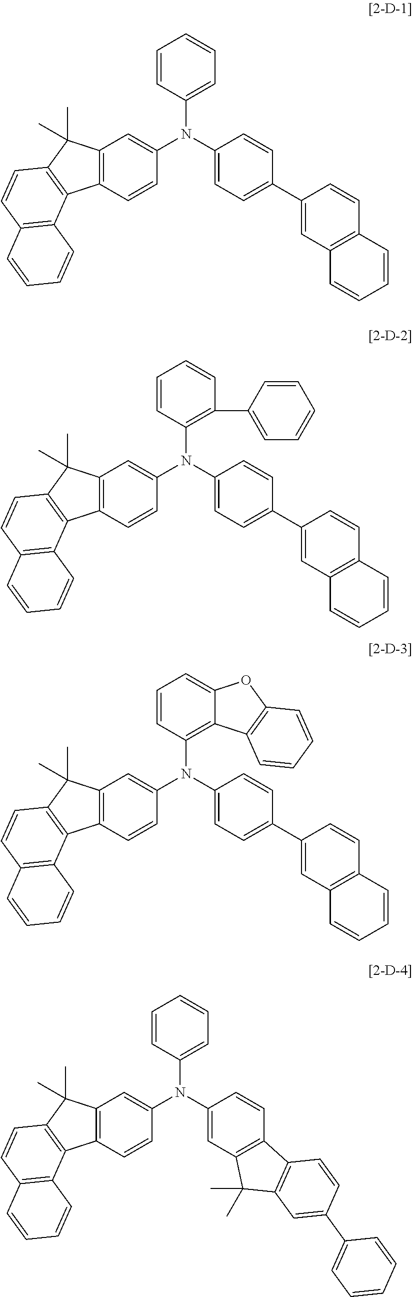 [SMILES: CC1(C)c2cc(N(c3ccccc3)c3ccc(-c4ccc5ccccc5c4)cc3)ccc2-c2c1ccc1ccccc21 ; CC1(C)c2cc(N(c3ccc(-c4ccc5ccccc5c4)cc3)c3ccccc3-c3ccccc3)ccc2-c2c1ccc1ccccc21 ; CC1(C)c2cc(N(c3ccc(-c4ccc5ccccc5c4)cc3)c3cccc4oc5ccccc5c34)ccc2-c2c1ccc1ccccc21 ; CC1(C)c2cc(-c3ccccc3)ccc2-c2ccc(N(c3ccccc3)c3ccc4c(c3)C(C)(C)c3ccc5ccccc5c3-4)cc21]

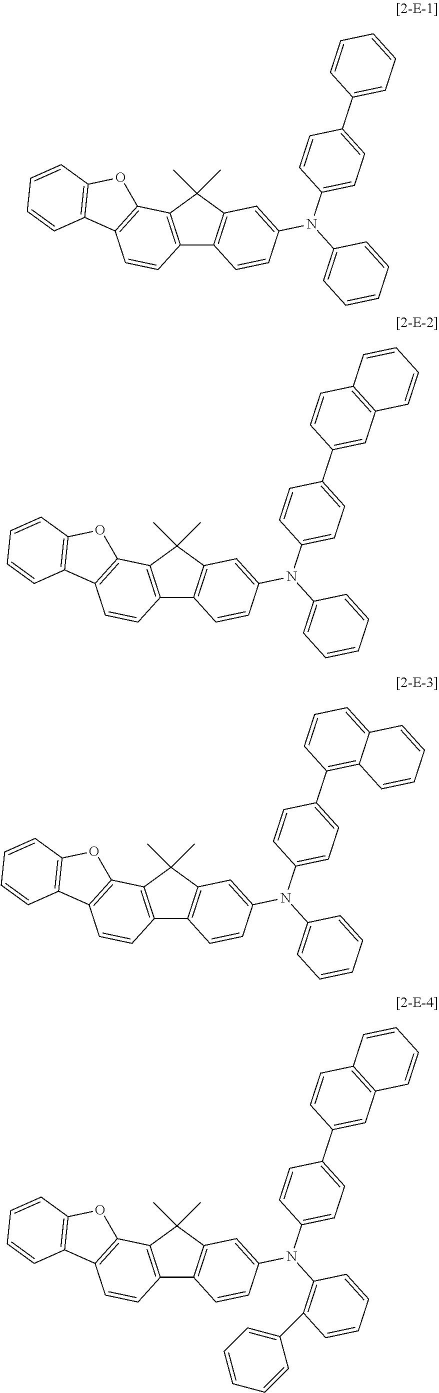 [SMILES: CC1(C)c2cc(N(c3ccccc3)c3ccc(-c4ccccc4)cc3)ccc2-c2ccc3c(oc4ccccc43)c21 ; CC1(C)c2cc(N(c3ccccc3)c3ccc(-c4ccc5ccccc5c4)cc3)ccc2-c2ccc3c(oc4ccccc43)c21 ; CC1(C)c2cc(N(c3ccccc3)c3ccc(-c4cccc5ccccc45)cc3)ccc2-c2ccc3c(oc4ccccc43)c21 ; CC1(C)c2cc(N(c3ccc(-c4ccc5ccccc5c4)cc3)c3ccccc3-c3ccccc3)ccc2-c2ccc3c(oc4ccccc43)c21]

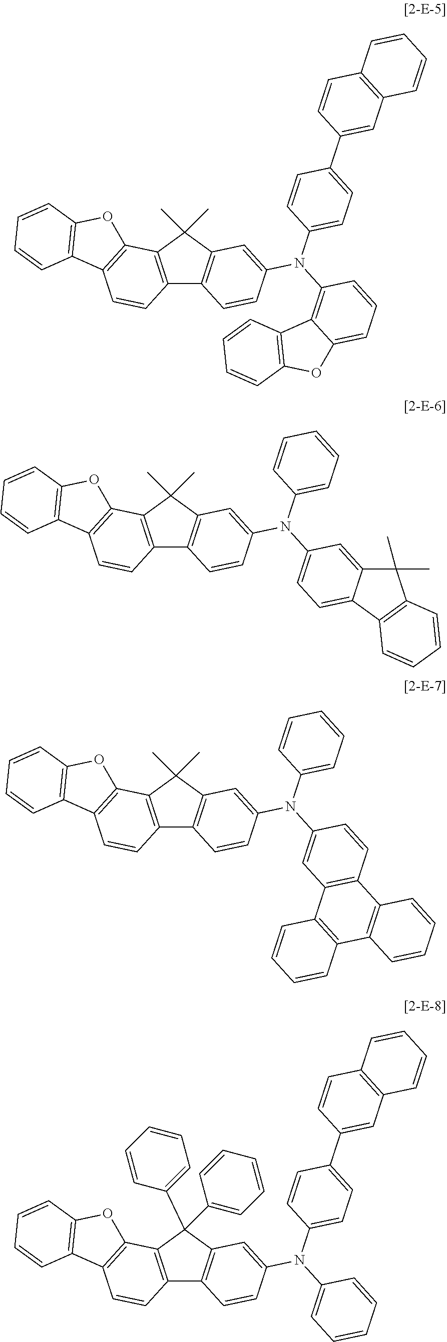 [SMILES: CC1(C)c2cc(N(c3ccc(-c4ccc5ccccc5c4)cc3)c3cccc4oc5ccccc5c34)ccc2-c2ccc3c(oc4ccccc43)c21 ; CC1(C)c2ccccc2-c2ccc(N(c3ccccc3)c3ccc4c(c3)C(C)(C)c3c-4ccc4c3oc3ccccc34)cc21 ; CC1(C)c2cc(N(c3ccccc3)c3ccc4c5ccccc5c5ccccc5c4c3)ccc2-c2ccc3c(oc4ccccc43)c21 ; c1ccc(N(c2ccc(-c3ccc4ccccc4c3)cc2)c2ccc3c(c2)C(c2ccccc2)(c2ccccc2)c2c-3ccc3c2oc2ccccc23)cc1]

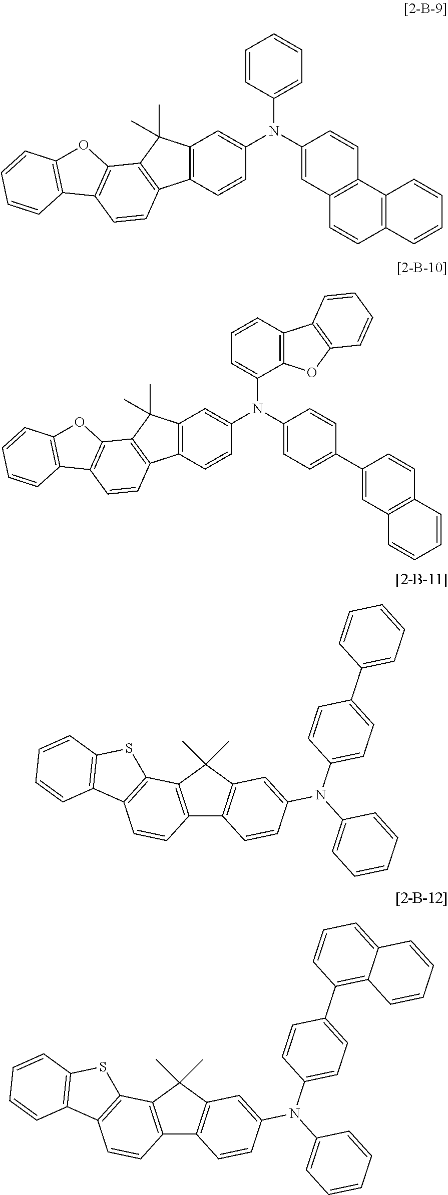 [SMILES: CC1(C)c2cc(N(c3ccccc3)c3ccc4c(ccc5ccccc54)c3)ccc2-c2ccc3c(oc4ccccc43)c21 ; CC1(C)c2cc(N(c3ccc(-c4ccc5ccccc5c4)cc3)c3cccc4c3oc3ccccc34)ccc2-c2ccc3c(oc4ccccc43)c21 ; CC1(C)c2cc(N(c3ccccc3)c3ccc(-c4ccccc4)cc3)ccc2-c2ccc3c(sc4ccccc43)c21 ; CC1(C)c2cc(N(c3ccccc3)c3ccc(-c4cccc5ccccc45)cc3)ccc2-c2ccc3c(sc4ccccc43)c21]

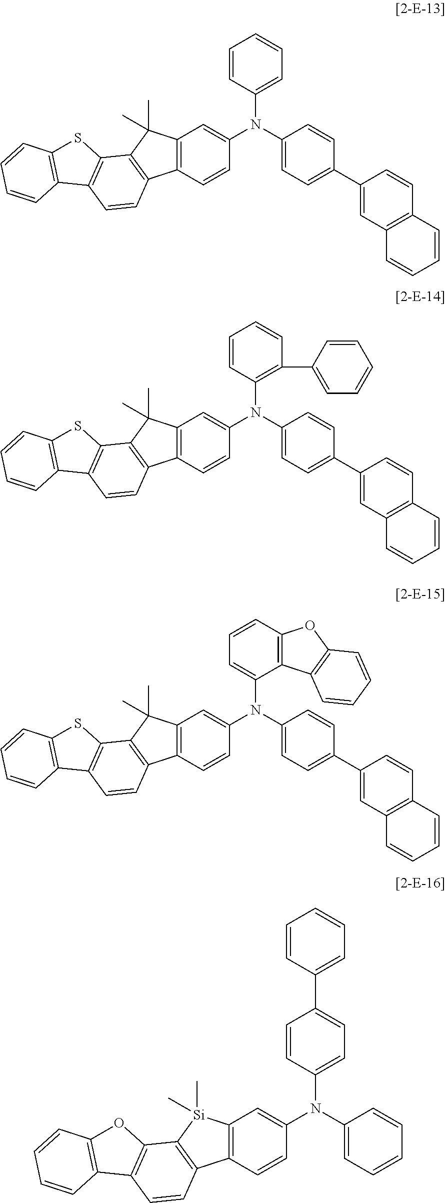 [SMILES: CC1(C)c2cc(N(c3ccccc3)c3ccc(-c4ccc5ccccc5c4)cc3)ccc2-c2ccc3c(sc4ccccc43)c21 ; CC1(C)c2cc(N(c3ccc(-c4ccc5ccccc5c4)cc3)c3ccccc3-c3ccccc3)ccc2-c2ccc3c(sc4ccccc43)c21 ; CC1(C)c2cc(N(c3ccc(-c4ccc5ccccc5c4)cc3)c3cccc4oc5ccccc5c34)ccc2-c2ccc3c(sc4ccccc43)c21 ; C[Si]1(C)c2cc(N(c3ccccc3)c3ccc(-c4ccccc4)cc3)ccc2-c2ccc3c(oc4ccccc43)c21]

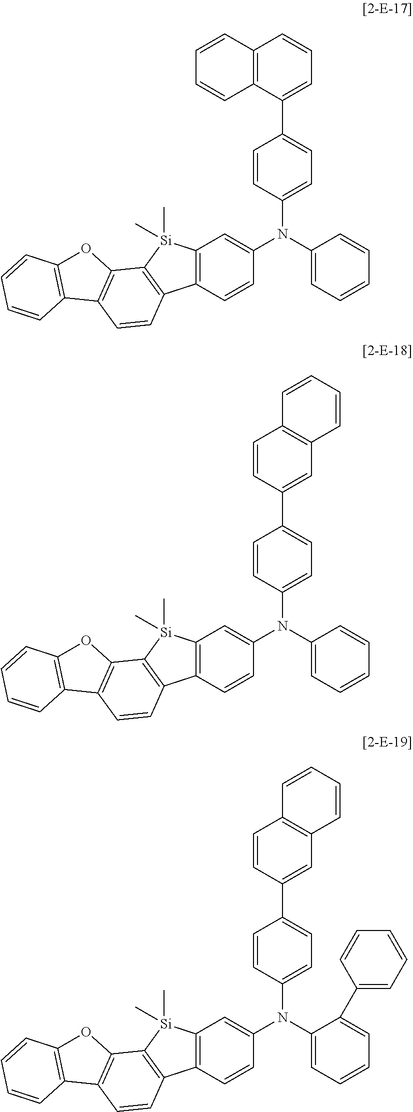 [SMILES: C[Si]1(C)c2cc(N(c3ccccc3)c3ccc(-c4cccc5ccccc45)cc3)ccc2-c2ccc3c(oc4ccccc43)c21 ; C[Si]1(C)c2cc(N(c3ccccc3)c3ccc(-c4ccc5ccccc5c4)cc3)ccc2-c2ccc3c(oc4ccccc43)c21 ; C[Si]1(C)c2cc(N(c3ccc(-c4ccc5ccccc5c4)cc3)c3ccccc3-c3ccccc3)ccc2-c2ccc3c(oc4ccccc43)c21]

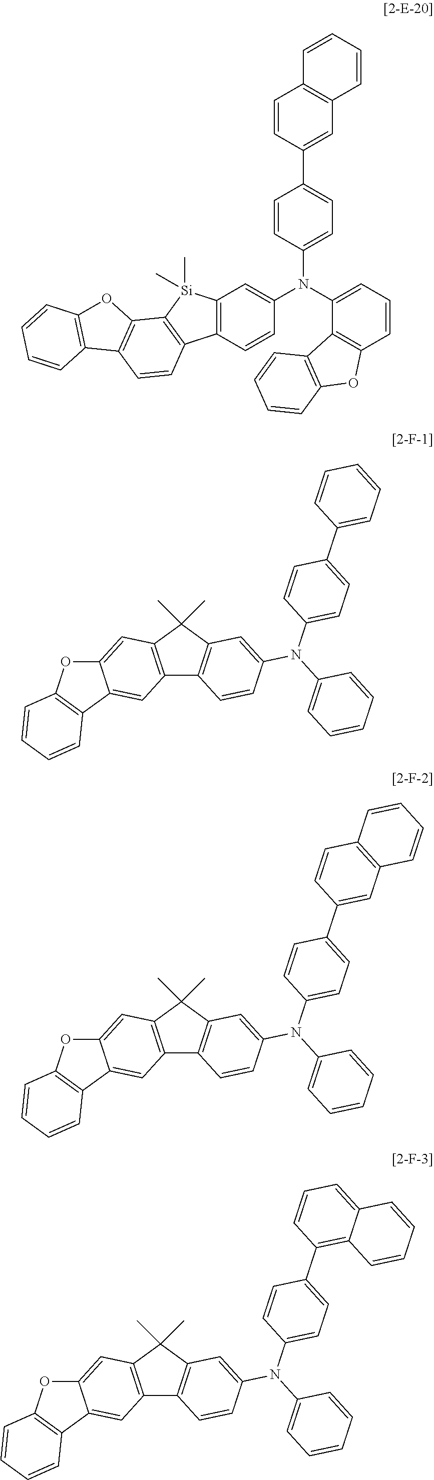 [SMILES: C[Si]1(C)c2cc(N(c3ccc(-c4ccc5ccccc5c4)cc3)c3cccc4oc5ccccc5c34)ccc2-c2ccc3c(oc4ccccc43)c21 ; CC1(C)c2cc(N(c3ccccc3)c3ccc(-c4ccccc4)cc3)ccc2-c2cc3c(cc21)oc1ccccc13 ; CC1(C)c2cc(N(c3ccccc3)c3ccc(-c4ccc5ccccc5c4)cc3)ccc2-c2cc3c(cc21)oc1ccccc13 ; CC1(C)c2cc(N(c3ccccc3)c3ccc(-c4cccc5ccccc45)cc3)ccc2-c2cc3c(cc21)oc1ccccc13]

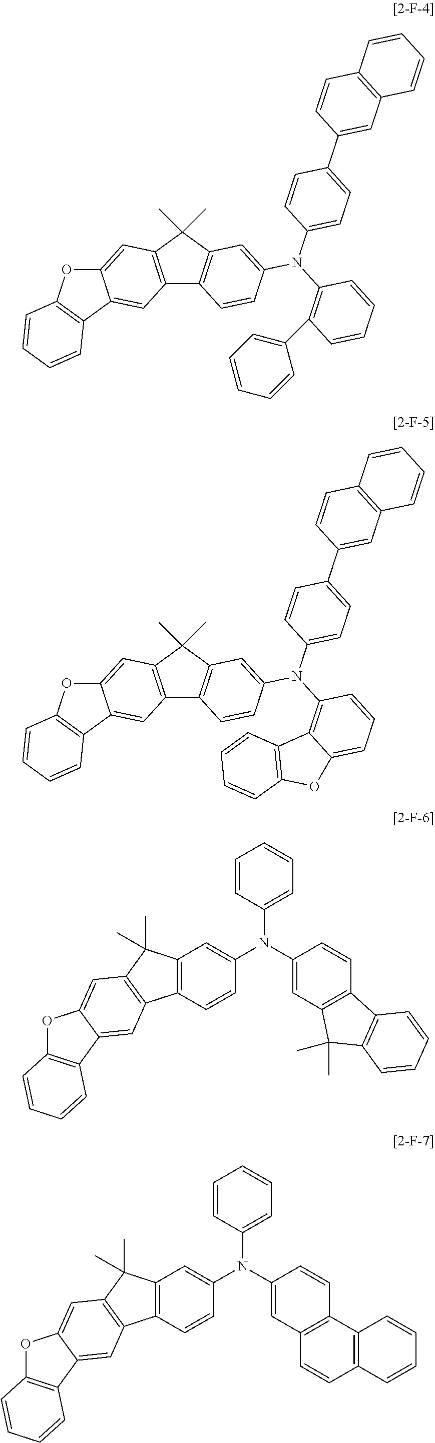

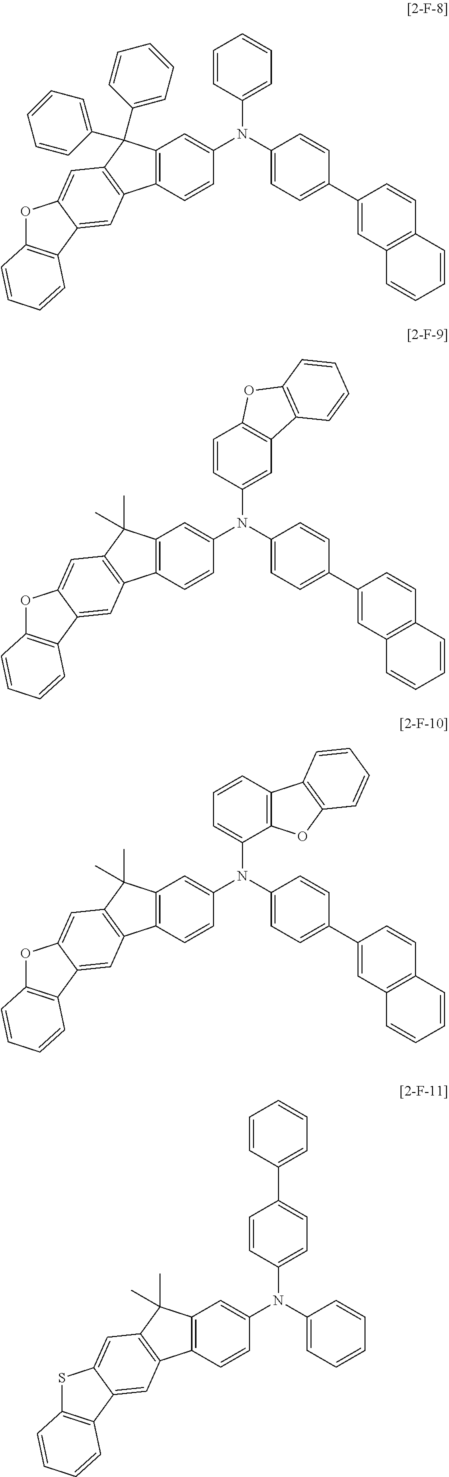 [SMILES: c1ccc(N(c2ccc(-c3ccc4ccccc4c3)cc2)c2ccc3c(c2)C(c2ccccc2)(c2ccccc2)c2cc4oc5ccccc5c4cc2-3)cc1 ; CC1(C)c2cc(N(c3ccc(-c4ccc5ccccc5c4)cc3)c3ccc4oc5ccccc5c4c3)ccc2-c2cc3c(cc21)oc1ccccc13 ; CC1(C)c2cc(N(c3ccc(-c4ccc5ccccc5c4)cc3)c3cccc4c3oc3ccccc34)ccc2-c2cc3c(cc21)oc1ccccc13 ; CC1(C)c2cc(N(c3ccccc3)c3ccc(-c4ccccc4)cc3)ccc2-c2cc3c(cc21)sc1ccccc13]

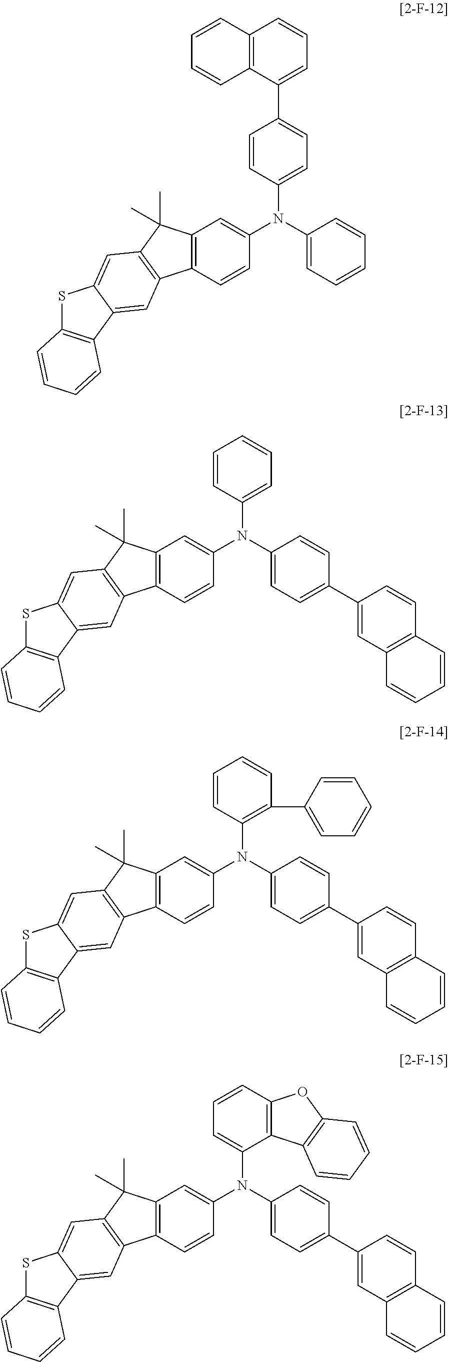 [SMILES: CC1(C)c2cc(N(c3ccccc3)c3ccc(-c4cccc5ccccc45)cc3)ccc2-c2cc3c(cc21)sc1ccccc13 ; CC1(C)c2cc(N(c3ccccc3)c3ccc(-c4ccc5ccccc5c4)cc3)ccc2-c2cc3c(cc21)sc1ccccc13 ; CC1(C)c2cc(N(c3ccc(-c4ccc5ccccc5c4)cc3)c3ccccc3-c3ccccc3)ccc2-c2cc3c(cc21)sc1ccccc13 ; CC1(C)c2cc(N(c3ccc(-c4ccc5ccccc5c4)cc3)c3cccc4oc5ccccc5c34)ccc2-c2cc3c(cc21)sc1ccccc13]

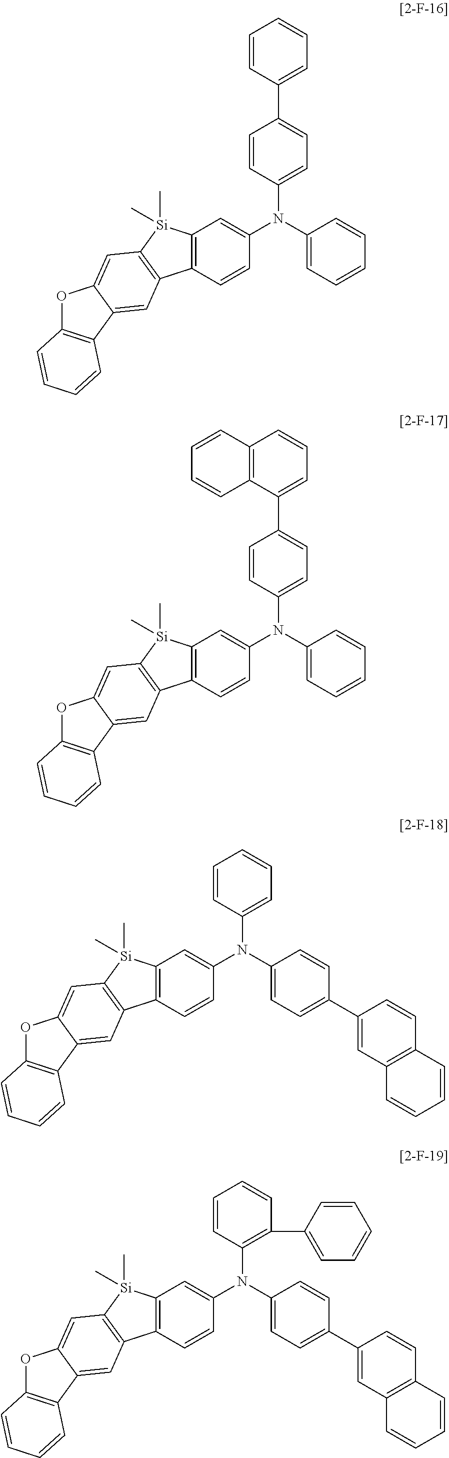 [SMILES: C[Si]1(C)c2cc(N(c3ccccc3)c3ccc(-c4ccccc4)cc3)ccc2-c2cc3c(cc21)oc1ccccc13 ; C[Si]1(C)c2cc(N(c3ccccc3)c3ccc(-c4cccc5ccccc45)cc3)ccc2-c2cc3c(cc21)oc1ccccc13 ; C[Si]1(C)c2cc(N(c3ccccc3)c3ccc(-c4ccc5ccccc5c4)cc3)ccc2-c2cc3c(cc21)oc1ccccc13 ; C[Si]1(C)c2cc(N(c3ccc(-c4ccc5ccccc5c4)cc3)c3ccccc3-c3ccccc3)ccc2-c2cc3c(cc21)oc1ccccc13]

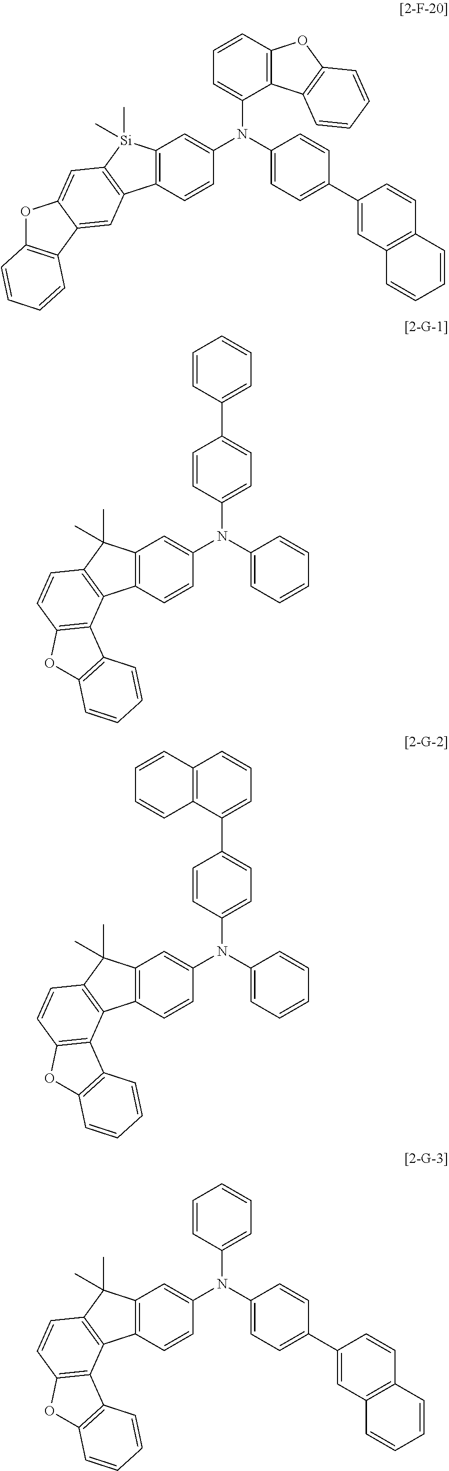 [SMILES: C[Si]1(C)c2cc(N(c3ccc(-c4ccc5ccccc5c4)cc3)c3cccc4oc5ccccc5c34)ccc2-c2cc3c(cc21)oc1ccccc13 ; CC1(C)c2cc(N(c3ccccc3)c3ccc(-c4ccccc4)cc3)ccc2-c2c1ccc1oc3ccccc3c21 ; CC1(C)c2cc(N(c3ccccc3)c3ccc(-c4cccc5ccccc45)cc3)ccc2-c2c1ccc1oc3ccccc3c21 ; CC1(C)c2cc(N(c3ccccc3)c3ccc(-c4ccc5ccccc5c4)cc3)ccc2-c2c1ccc1oc3ccccc3c21]

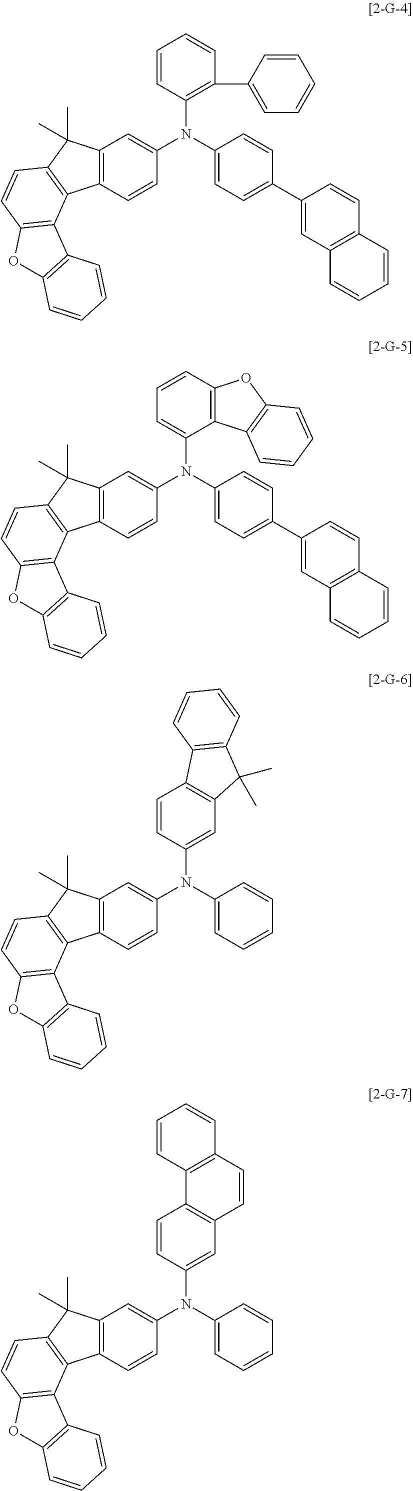 [SMILES: CC1(C)c2cc(N(c3ccc(-c4ccc5ccccc5c4)cc3)c3ccccc3-c3ccccc3)ccc2-c2c1ccc1oc3ccccc3c21 ; CC1(C)c2cc(N(c3ccc(-c4ccc5ccccc5c4)cc3)c3cccc4oc5ccccc5c34)ccc2-c2c1ccc1oc3ccccc3c21 ; CC1(C)c2ccccc2-c2ccc(N(c3ccccc3)c3ccc4c(c3)C(C)(C)c3ccc5oc6ccccc6c5c3-4)cc21 ; CC1(C)c2cc(N(c3ccccc3)c3ccc4c(ccc5ccccc54)c3)ccc2-c2c1ccc1oc3ccccc3c21]

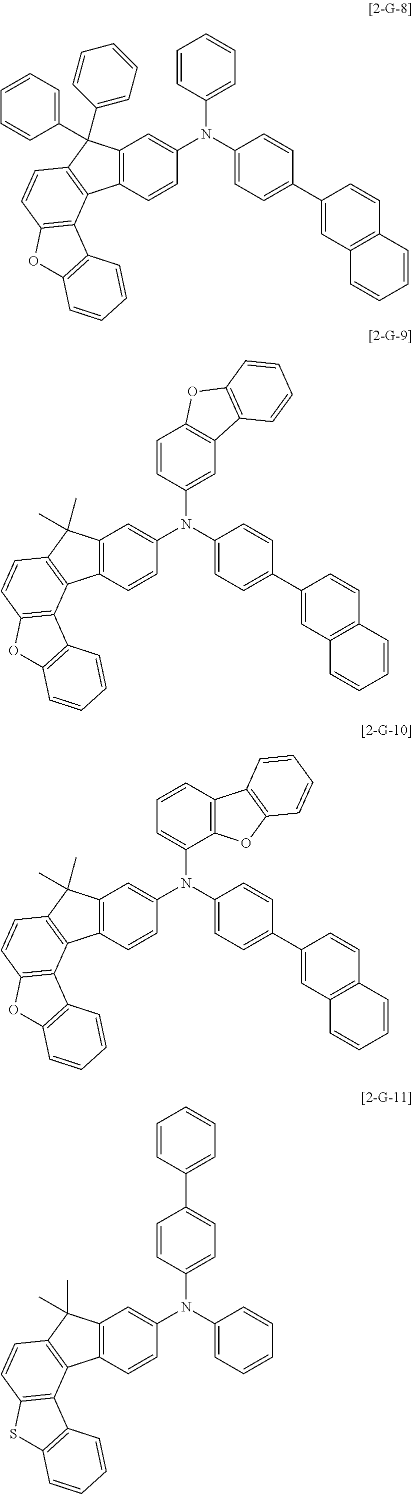 [SMILES: c1ccc(N(c2ccc(-c3ccc4ccccc4c3)cc2)c2ccc3c(c2)C(c2ccccc2)(c2ccccc2)c2ccc4oc5ccccc5c4c2-3)cc1 ; CC1(C)c2cc(N(c3ccc(-c4ccc5ccccc5c4)cc3)c3ccc4oc5ccccc5c4c3)ccc2-c2c1ccc1oc3ccccc3c21 ; CC1(C)c2cc(N(c3ccc(-c4ccc5ccccc5c4)cc3)c3cccc4c3oc3ccccc34)ccc2-c2c1ccc1oc3ccccc3c21 ; CC1(C)c2cc(N(c3ccccc3)c3ccc(-c4ccccc4)cc3)ccc2-c2c1ccc1sc3ccccc3c21]

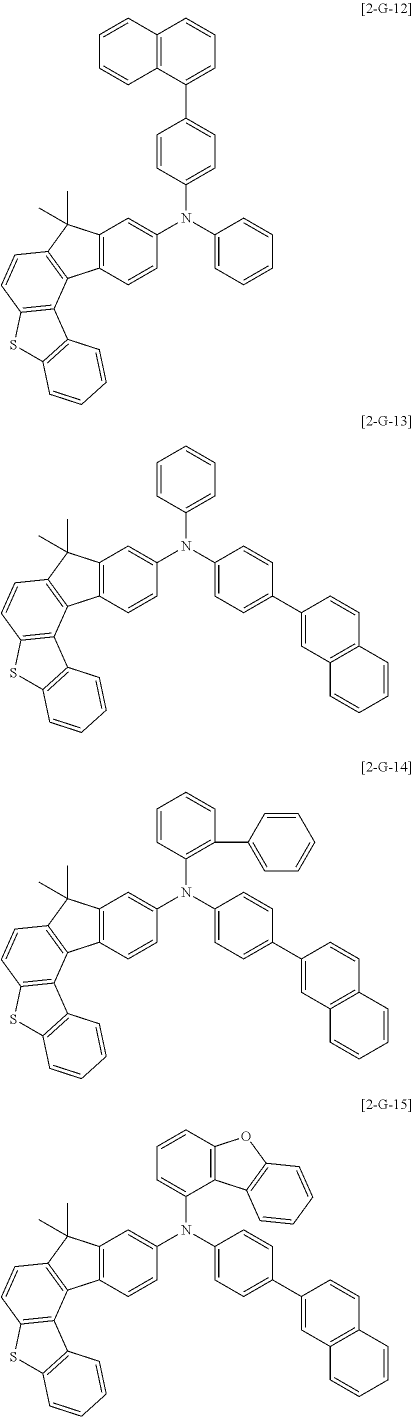 [SMILES: CC1(C)c2cc(N(c3ccccc3)c3ccc(-c4cccc5ccccc45)cc3)ccc2-c2c1ccc1sc3ccccc3c21 ; CC1(C)c2cc(N(c3ccccc3)c3ccc(-c4ccc5ccccc5c4)cc3)ccc2-c2c1ccc1sc3ccccc3c21 ; CC1(C)c2cc(N(c3ccc(-c4ccc5ccccc5c4)cc3)c3ccccc3-c3ccccc3)ccc2-c2c1ccc1sc3ccccc3c21 ; CC1(C)c2cc(N(c3ccc(-c4ccc5ccccc5c4)cc3)c3cccc4oc5ccccc5c34)ccc2-c2c1ccc1sc3ccccc3c21]

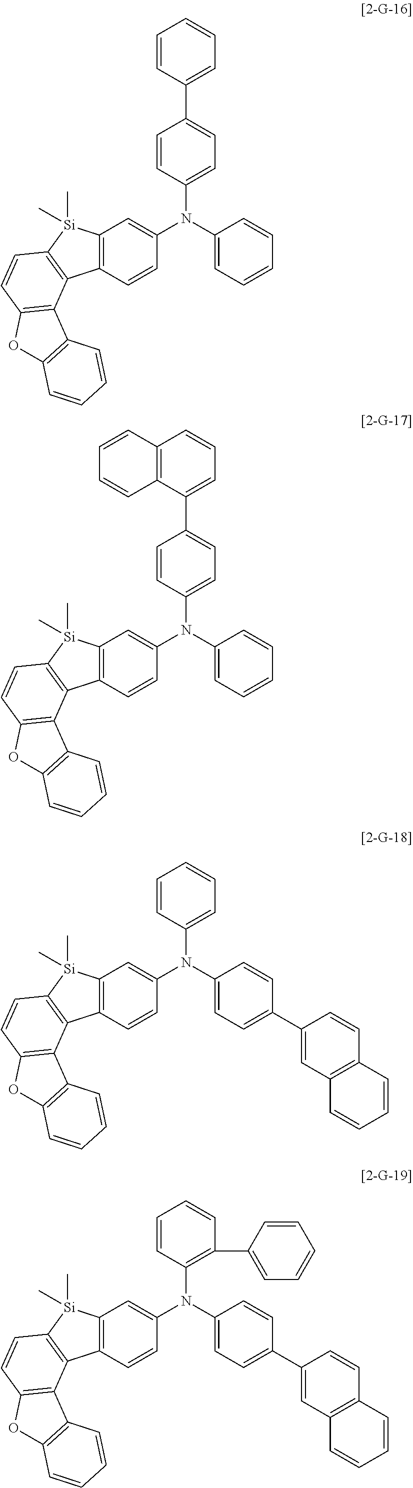

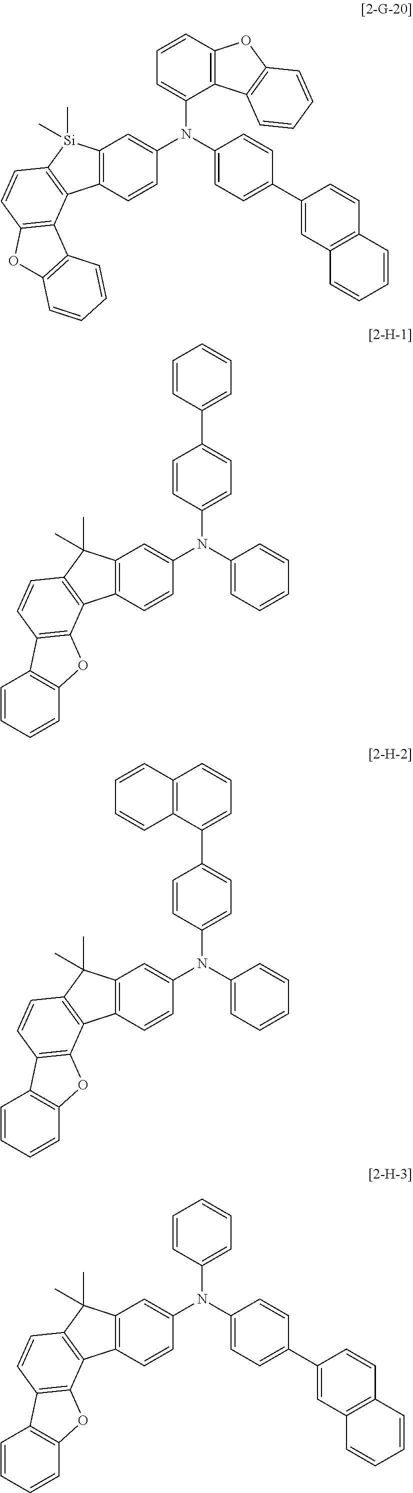 [SMILES: C[Si]1(C)c2cc(N(c3ccc(-c4ccc5ccccc5c4)cc3)c3cccc4oc5ccccc5c34)ccc2-c2c1ccc1oc3ccccc3c21 ; CC1(C)c2cc(N(c3ccccc3)c3ccc(-c4ccccc4)cc3)ccc2-c2c1ccc1c2oc2ccccc21 ; CC1(C)c2cc(N(c3ccccc3)c3ccc(-c4cccc5ccccc45)cc3)ccc2-c2c1ccc1c2oc2ccccc21 ; CC1(C)c2cc(N(c3ccccc3)c3ccc(-c4ccc5ccccc5c4)cc3)ccc2-c2c1ccc1c2oc2ccccc21]

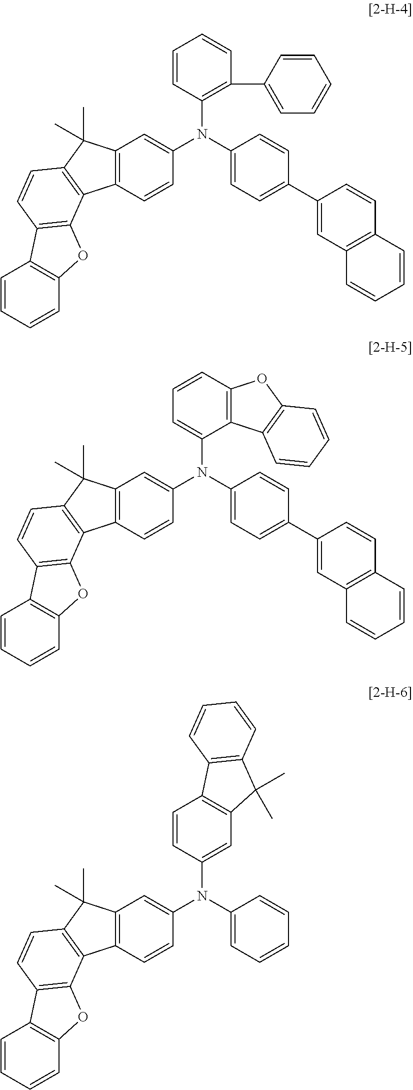 [SMILES: CC1(C)c2cc(N(c3ccc(-c4ccc5ccccc5c4)cc3)c3ccccc3-c3ccccc3)ccc2-c2c1ccc1c2oc2ccccc21 ; CC1(C)c2cc(N(c3ccc(-c4ccc5ccccc5c4)cc3)c3cccc4oc5ccccc5c34)ccc2-c2c1ccc1c2oc2ccccc21 ; CC1(C)c2ccccc2-c2ccc(N(c3ccccc3)c3ccc4c(c3)C(C)(C)c3ccc5c(oc6ccccc65)c3-4)cc21]

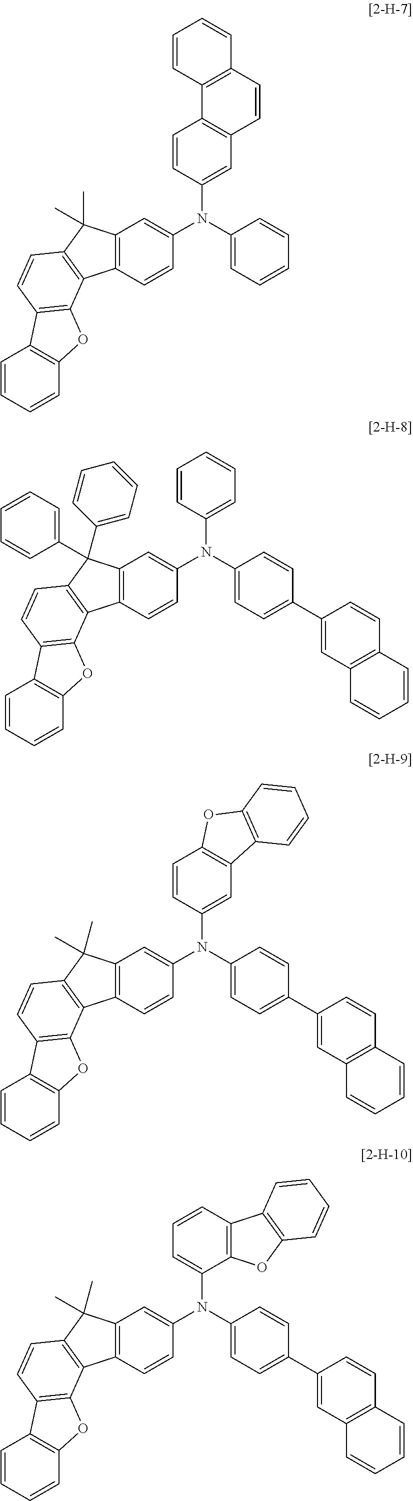 [SMILES: CC1(C)c2cc(N(c3ccccc3)c3ccc4c(ccc5ccccc54)c3)ccc2-c2c1ccc1c2oc2ccccc21 ; c1ccc(N(c2ccc(-c3ccc4ccccc4c3)cc2)c2ccc3c(c2)C(c2ccccc2)(c2ccccc2)c2ccc4c(oc5ccccc54)c2-3)cc1 ; CC1(C)c2cc(N(c3ccc(-c4ccc5ccccc5c4)cc3)c3ccc4oc5ccccc5c4c3)ccc2-c2c1ccc1c2oc2ccccc21 ; CC1(C)c2cc(N(c3ccc(-c4ccc5ccccc5c4)cc3)c3cccc4c3oc3ccccc34)ccc2-c2c1ccc1c2oc2ccccc21]

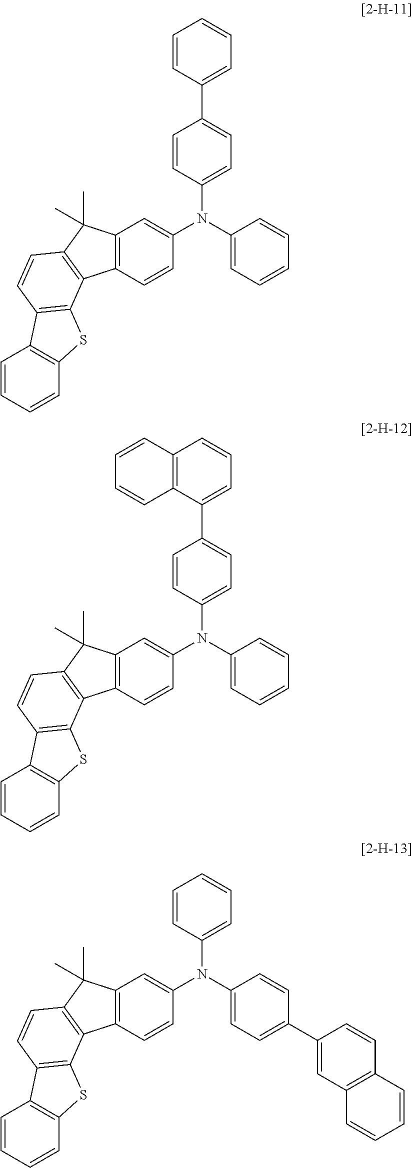 [SMILES: CC1(C)c2cc(N(c3ccccc3)c3ccc(-c4ccccc4)cc3)ccc2-c2c1ccc1c2sc2ccccc21 ; CC1(C)c2cc(N(c3ccccc3)c3ccc(-c4cccc5ccccc45)cc3)ccc2-c2c1ccc1c2sc2ccccc21 ; CC1(C)c2cc(N(c3ccccc3)c3ccc(-c4ccc5ccccc5c4)cc3)ccc2-c2c1ccc1c2sc2ccccc21]

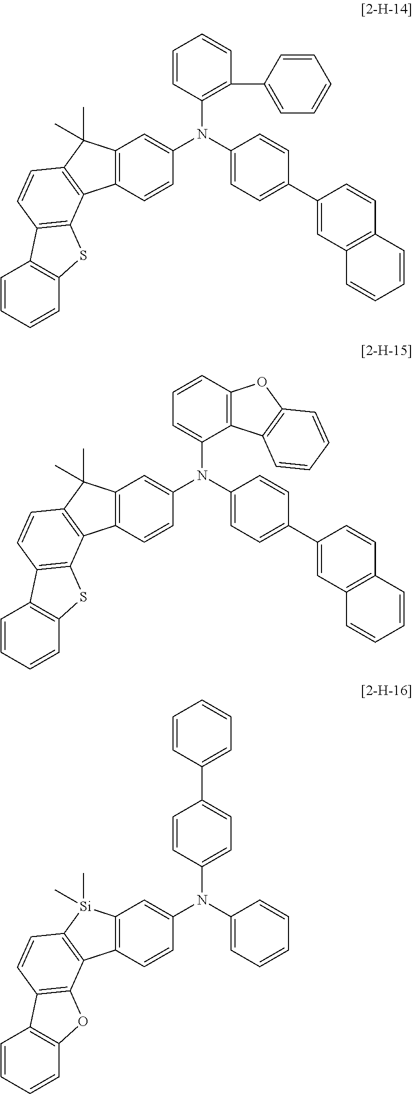 [SMILES: CC1(C)c2cc(N(c3ccc(-c4ccc5ccccc5c4)cc3)c3ccccc3-c3ccccc3)ccc2-c2c1ccc1c2sc2ccccc21 ; CC1(C)c2cc(N(c3ccc(-c4ccc5ccccc5c4)cc3)c3cccc4oc5ccccc5c34)ccc2-c2c1ccc1c2sc2ccccc21 ; C[Si]1(C)c2cc(N(c3ccccc3)c3ccc(-c4ccccc4)cc3)ccc2-c2c1ccc1c2oc2ccccc21]

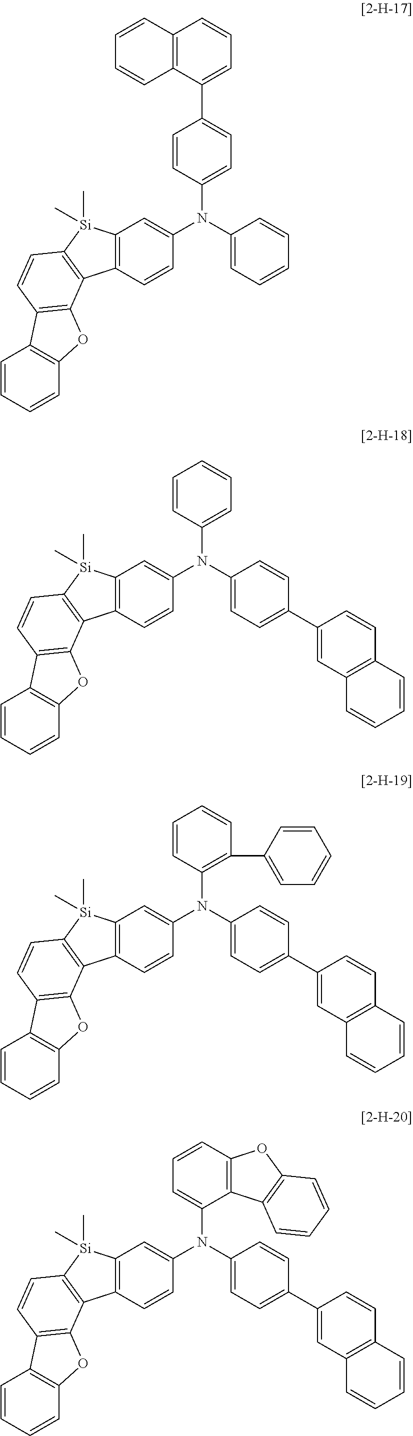 [SMILES: C[Si]1(C)c2cc(N(c3ccccc3)c3ccc(-c4cccc5ccccc45)cc3)ccc2-c2c1ccc1c2oc2ccccc21 ; C[Si]1(C)c2cc(N(c3ccccc3)c3ccc(-c4ccc5ccccc5c4)cc3)ccc2-c2c1ccc1c2oc2ccccc21 ; C[Si]1(C)c2cc(N(c3ccc(-c4ccc5ccccc5c4)cc3)c3ccccc3-c3ccccc3)ccc2-c2c1ccc1c2oc2ccccc21 ; C[Si]1(C)c2cc(N(c3ccc(-c4ccc5ccccc5c4)cc3)c3cccc4oc5ccccc5c34)ccc2-c2c1ccc1c2oc2ccccc21]

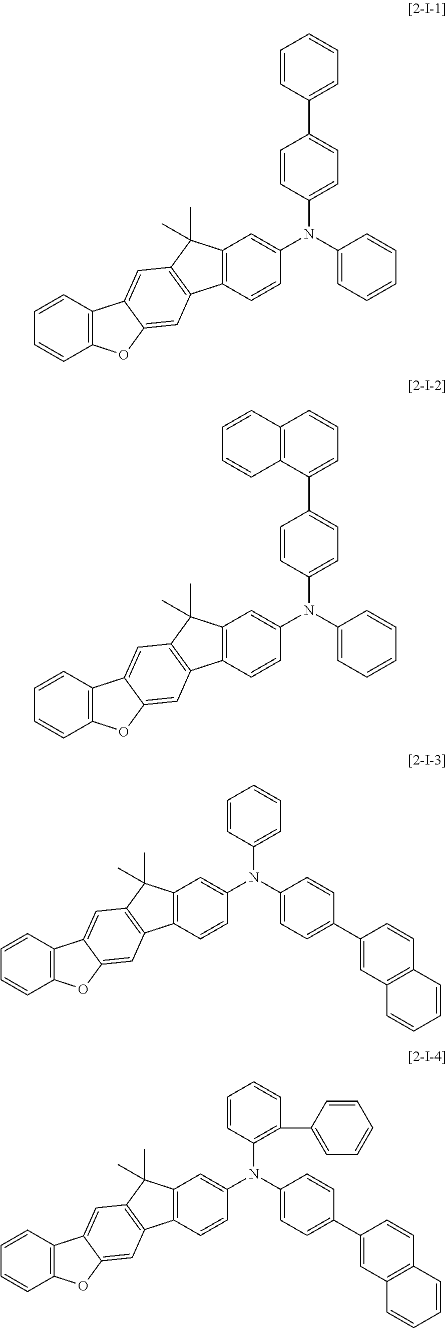 [SMILES: CC1(C)c2cc(N(c3ccccc3)c3ccc(-c4ccccc4)cc3)ccc2-c2cc3oc4ccccc4c3cc21 ; CC1(C)c2cc(N(c3ccccc3)c3ccc(-c4cccc5ccccc45)cc3)ccc2-c2cc3oc4ccccc4c3cc21 ; CC1(C)c2cc(N(c3ccccc3)c3ccc(-c4ccc5ccccc5c4)cc3)ccc2-c2cc3oc4ccccc4c3cc21 ; CC1(C)c2cc(N(c3ccc(-c4ccc5ccccc5c4)cc3)c3ccccc3-c3ccccc3)ccc2-c2cc3oc4ccccc4c3cc21]

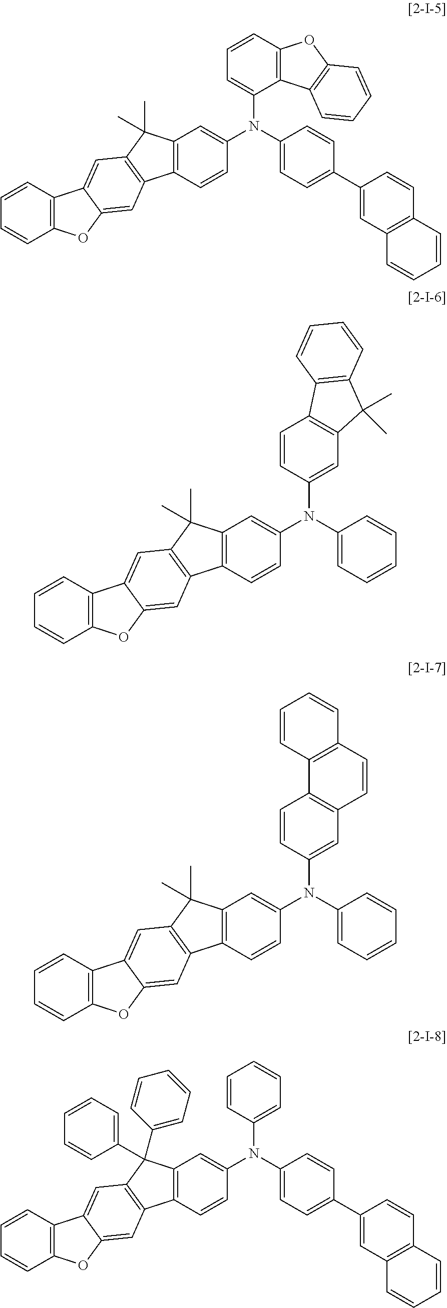 [SMILES: CC1(C)c2cc(N(c3ccc(-c4ccc5ccccc5c4)cc3)c3cccc4oc5ccccc5c34)ccc2-c2cc3oc4ccccc4c3cc21 ; CC1(C)c2ccccc2-c2ccc(N(c3ccccc3)c3ccc4c(c3)C(C)(C)c3cc5c(cc3-4)oc3ccccc35)cc21 ; CC1(C)c2cc(N(c3ccccc3)c3ccc4c(ccc5ccccc54)c3)ccc2-c2cc3oc4ccccc4c3cc21 ; c1ccc(N(c2ccc(-c3ccc4ccccc4c3)cc2)c2ccc3c(c2)C(c2ccccc2)(c2ccccc2)c2cc4c(cc2-3)oc2ccccc24)cc1]

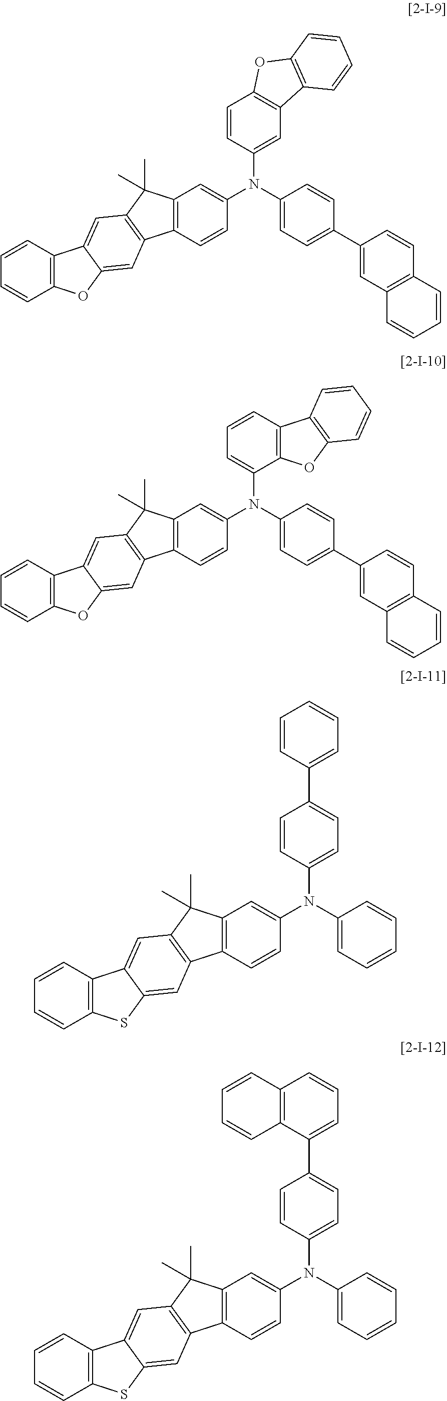 [SMILES: CC1(C)c2cc(N(c3ccc(-c4ccc5ccccc5c4)cc3)c3ccc4oc5ccccc5c4c3)ccc2-c2cc3oc4ccccc4c3cc21 ; CC1(C)c2cc(N(c3ccc(-c4ccc5ccccc5c4)cc3)c3cccc4c3oc3ccccc34)ccc2-c2cc3oc4ccccc4c3cc21 ; CC1(C)c2cc(N(c3ccccc3)c3ccc(-c4ccccc4)cc3)ccc2-c2cc3sc4ccccc4c3cc21 ; CC1(C)c2cc(N(c3ccccc3)c3ccc(-c4cccc5ccccc45)cc3)ccc2-c2cc3sc4ccccc4c3cc21]

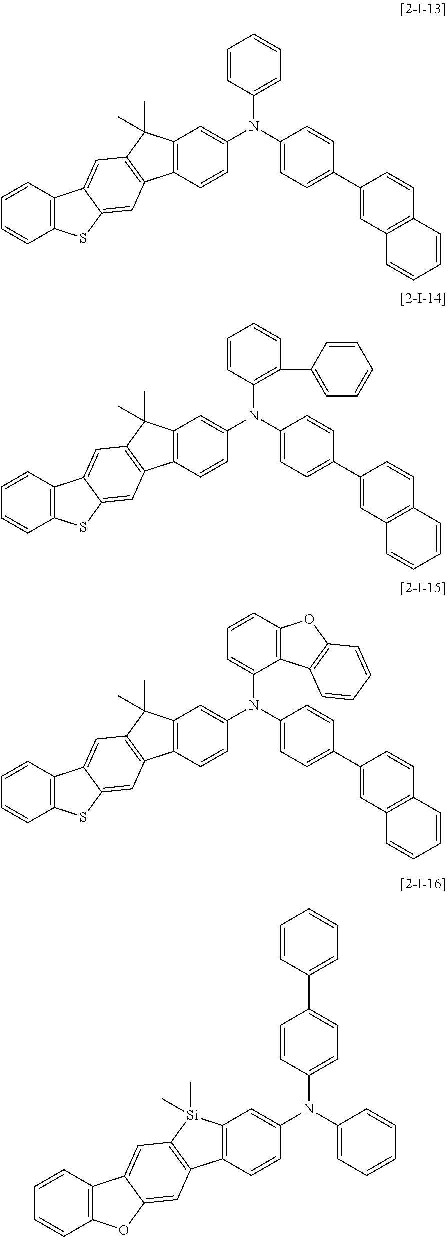 [SMILES: CC1(C)c2cc(N(c3ccccc3)c3ccc(-c4ccc5ccccc5c4)cc3)ccc2-c2cc3sc4ccccc4c3cc21 ; CC1(C)c2cc(N(c3ccc(-c4ccc5ccccc5c4)cc3)c3ccccc3-c3ccccc3)ccc2-c2cc3sc4ccccc4c3cc21 ; CC1(C)c2cc(N(c3ccc(-c4ccc5ccccc5c4)cc3)c3cccc4oc5ccccc5c34)ccc2-c2cc3sc4ccccc4c3cc21 ; C[Si]1(C)c2cc(N(c3ccccc3)c3ccc(-c4ccccc4)cc3)ccc2-c2cc3oc4ccccc4c3cc21]

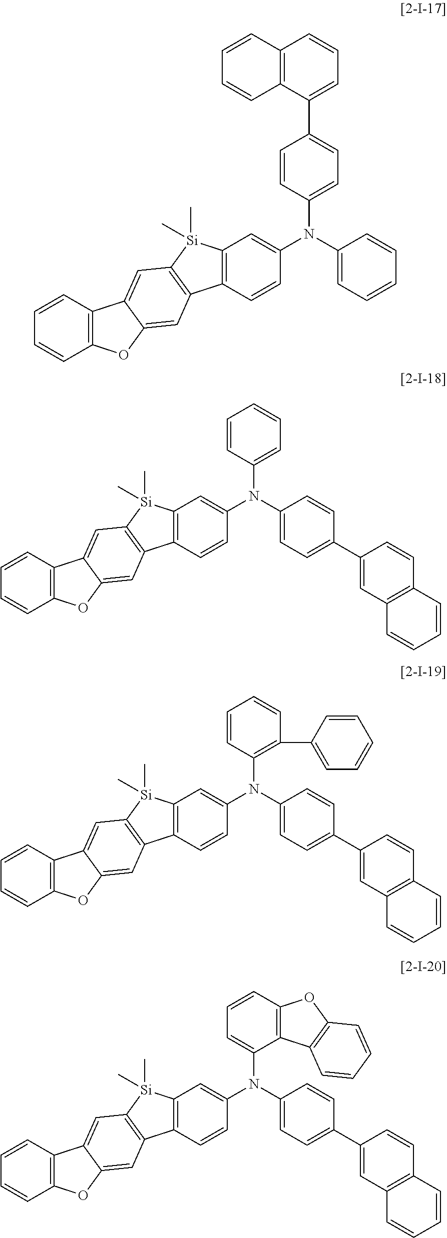 [SMILES: C[Si]1(C)c2cc(N(c3ccccc3)c3ccc(-c4cccc5ccccc45)cc3)ccc2-c2cc3oc4ccccc4c3cc21 ; C[Si]1(C)c2cc(N(c3ccccc3)c3ccc(-c4ccc5ccccc5c4)cc3)ccc2-c2cc3oc4ccccc4c3cc21 ; C[Si]1(C)c2cc(N(c3ccc(-c4ccc5ccccc5c4)cc3)c3ccccc3-c3ccccc3)ccc2-c2cc3oc4ccccc4c3cc21 ; C[Si]1(C)c2cc(N(c3ccc(-c4ccc5ccccc5c4)cc3)c3cccc4oc5ccccc5c34)ccc2-c2cc3oc4ccccc4c3cc21]

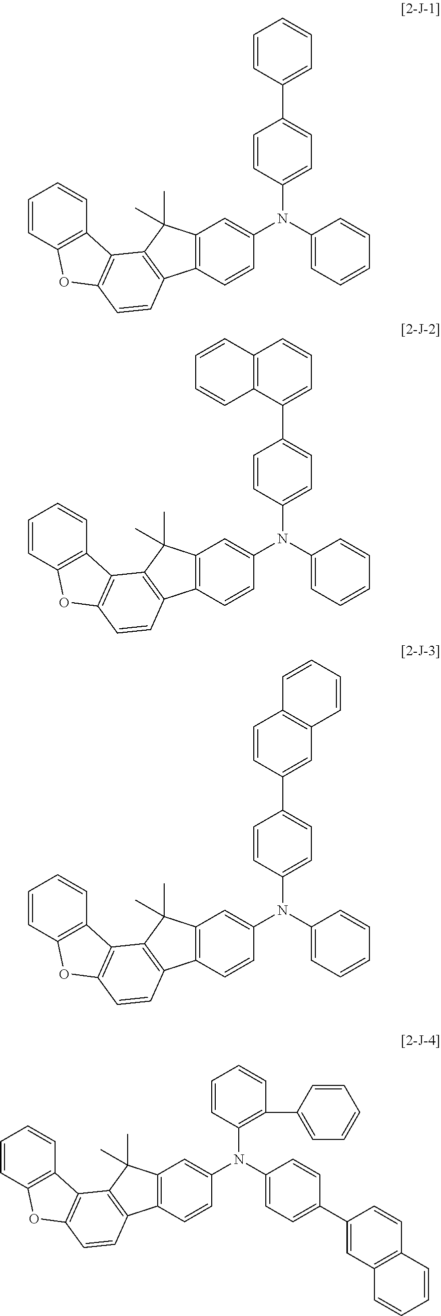 [SMILES: CC1(C)c2cc(N(c3ccccc3)c3ccc(-c4ccccc4)cc3)ccc2-c2ccc3oc4ccccc4c3c21 ; CC1(C)c2cc(N(c3ccccc3)c3ccc(-c4cccc5ccccc45)cc3)ccc2-c2ccc3oc4ccccc4c3c21 ; CC1(C)c2cc(N(c3ccccc3)c3ccc(-c4ccc5ccccc5c4)cc3)ccc2-c2ccc3oc4ccccc4c3c21 ; CC1(C)c2cc(N(c3ccc(-c4ccc5ccccc5c4)cc3)c3ccccc3-c3ccccc3)ccc2-c2ccc3oc4ccccc4c3c21]

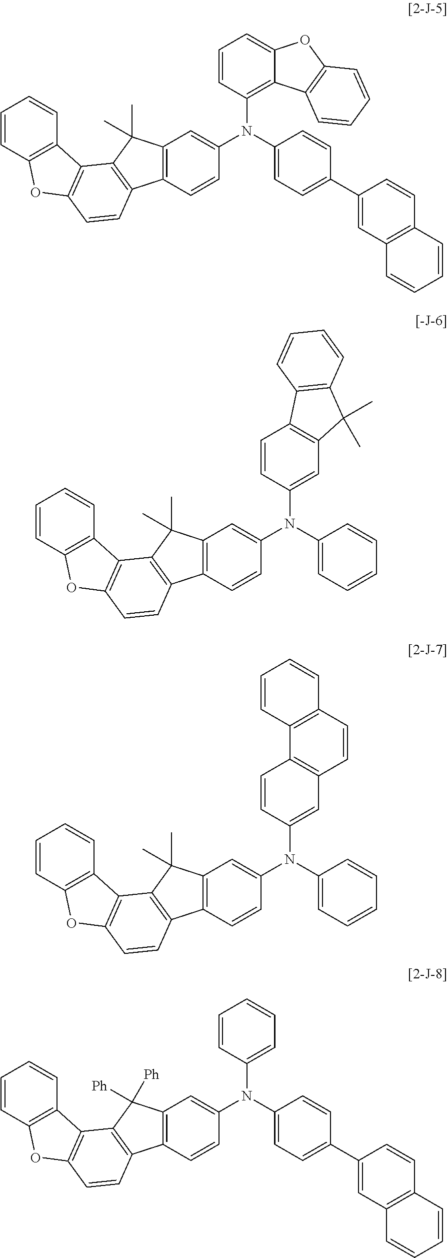 [SMILES: CC1(C)c2cc(N(c3ccc(-c4ccc5ccccc5c4)cc3)c3cccc4oc5ccccc5c34)ccc2-c2ccc3oc4ccccc4c3c21 ; CC1(C)c2ccccc2-c2ccc(N(c3ccccc3)c3ccc4c(c3)C(C)(C)c3c-4ccc4oc5ccccc5c34)cc21 ; CC1(C)c2cc(N(c3ccccc3)c3ccc4c(ccc5ccccc54)c3)ccc2-c2ccc3oc4ccccc4c3c21 ; c1ccc(N(c2ccc(-c3ccc4ccccc4c3)cc2)c2ccc3c(c2)C(c2ccccc2)(c2ccccc2)c2c-3ccc3oc4ccccc4c23)cc1]

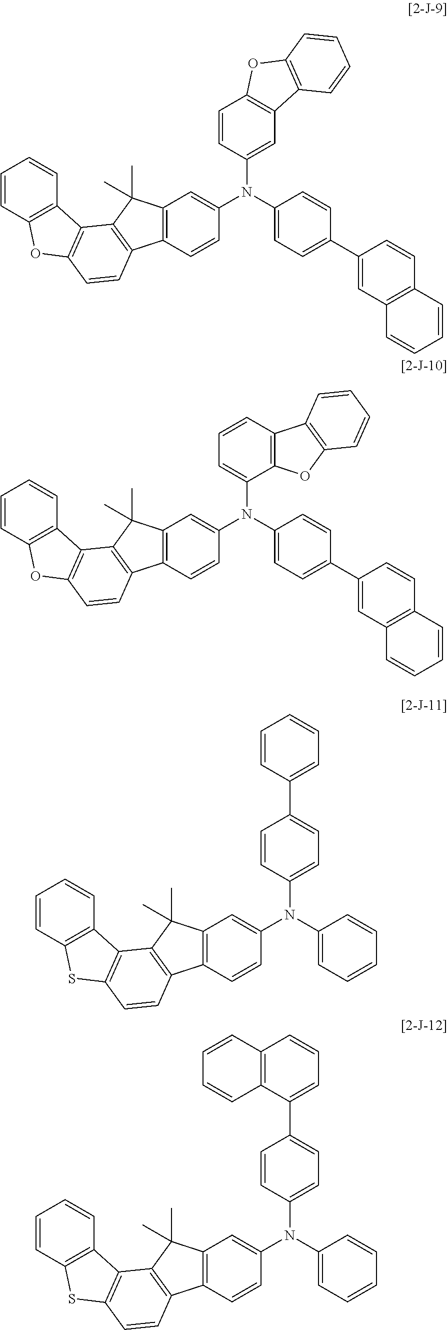 [SMILES: CC1(C)c2cc(N(c3ccc(-c4ccc5ccccc5c4)cc3)c3ccc4oc5ccccc5c4c3)ccc2-c2ccc3oc4ccccc4c3c21 ; CC1(C)c2cc(N(c3ccc(-c4ccc5ccccc5c4)cc3)c3cccc4c3oc3ccccc34)ccc2-c2ccc3oc4ccccc4c3c21 ; CC1(C)c2cc(N(c3ccccc3)c3ccc(-c4ccccc4)cc3)ccc2-c2ccc3sc4ccccc4c3c21 ; CC1(C)c2cc(N(c3ccccc3)c3ccc(-c4cccc5ccccc45)cc3)ccc2-c2ccc3sc4ccccc4c3c21]

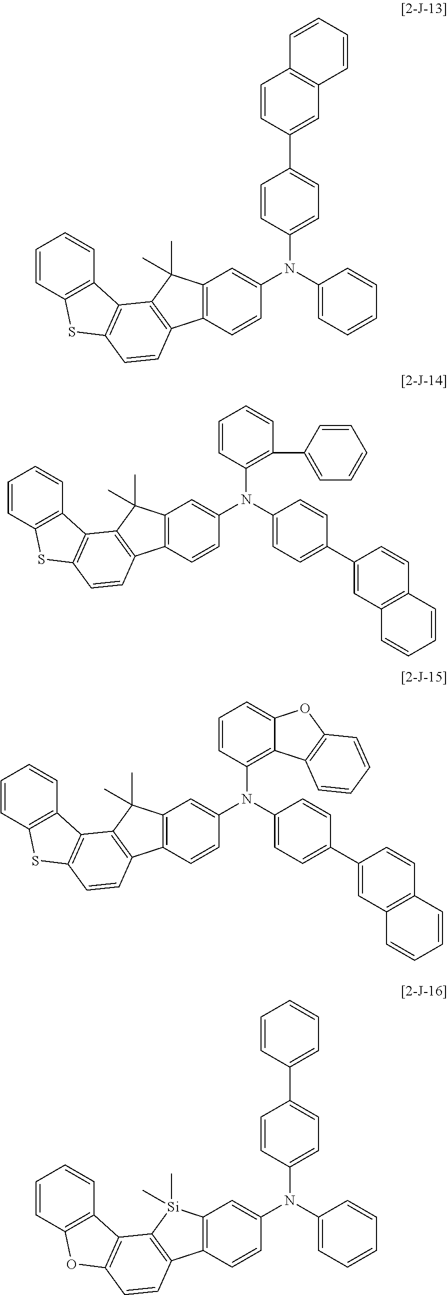 [SMILES: CC1(C)c2cc(N(c3ccccc3)c3ccc(-c4ccc5ccccc5c4)cc3)ccc2-c2ccc3sc4ccccc4c3c21 ; CC1(C)c2cc(N(c3ccc(-c4ccc5ccccc5c4)cc3)c3ccccc3-c3ccccc3)ccc2-c2ccc3sc4ccccc4c3c21 ; CC1(C)c2cc(N(c3ccc(-c4ccc5ccccc5c4)cc3)c3cccc4oc5ccccc5c34)ccc2-c2ccc3sc4ccccc4c3c21 ; C[Si]1(C)c2cc(N(c3ccccc3)c3ccc(-c4ccccc4)cc3)ccc2-c2ccc3oc4ccccc4c3c21]

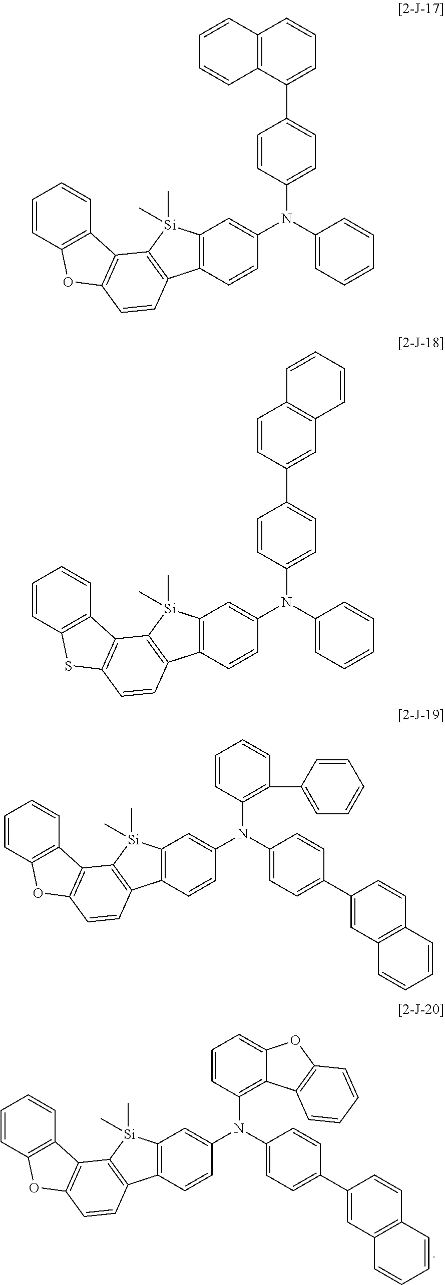 [SMILES: C[Si]1(C)c2cc(N(c3ccccc3)c3ccc(-c4cccc5ccccc45)cc3)ccc2-c2ccc3oc4ccccc4c3c21 ; C[Si]1(C)c2cc(N(c3ccccc3)c3ccc(-c4ccc5ccccc5c4)cc3)ccc2-c2ccc3sc4ccccc4c3c21 ; C[Si]1(C)c2cc(N(c3ccc(-c4ccc5ccccc5c4)cc3)c3ccccc3-c3ccccc3)ccc2-c2ccc3oc4ccccc4c3c21 ; C[Si]1(C)c2cc(N(c3ccc(-c4ccc5ccccc5c4)cc3)c3cccc4oc5ccccc5c34)ccc2-c2ccc3oc4ccccc4c3c21]

The first compound and the second compound may be included (e.g., mixed) in the composition, e.g., in a weight ratio of about 1:99 to about 99:1. Within the above range, an appropriate weight ratio may be adjusted using the electron transport capability of the first compound and the hole transport capability of the second compound to implement bipolar characteristics and to improve the efficiency and life-span. Within the above range, e.g., they may be included in a weight ratio of about 90:10 to about 10:90, about 80:20 to about 10:90, about 70:30 to about 10:90, about 60:40 to about 10:90 or about 60:40 to about 20:80. In an implementation, they may be included in a weight ratio of about 60:40 to about 30:70, e.g., about 60:40 or about 50:50.

In an implementation, the first compound and the second compound may each be included as a host of the light emitting layer, e.g., a phosphorescent host.

Hereinafter, an organic optoelectronic device including the aforementioned composition for an organic optoelectronic device is described.

The organic optoelectronic device may be a suitable device to convert electrical energy into photoenergy and vice versa, and may be, e.g., an organic photoelectric device, an organic light emitting diode, an organic solar cell, and an organic photoconductor drum.

Herein, an organic light emitting diode as one example of an organic optoelectronic device is described referring to drawings.

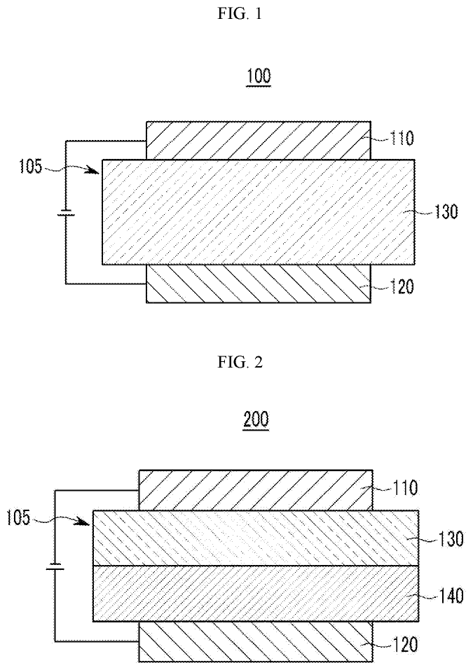

FIGS. 1 to 4 are cross-sectional views of organic light emitting diodes according to embodiments.

Referring to FIG. 1, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may include, e.g., a metal, a metal oxide or a conductive polymer. In an implementation, the anode 120 may include, e.g., a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may include, e.g., a metal, a metal oxide, or a conductive polymer. In an implementation, the cathode 110 may include, e.g., a metal such as magnesium, calcium, sodium, potassium, titanium, indium, yttrium, lithium, gadolinium, aluminum silver, tin, lead, cesium, barium, or the like, or an alloy thereof; a multi-layer structure material such as LiF/Al, LiO₂/Al, LiF/Ca, or BaF₂/Ca.

The organic layer 105 may include the aforementioned composition for an organic optoelectronic device.

The organic layer 105 may include the light emitting layer 130, and the light emitting layer 130 may include the aforementioned composition for an organic optoelectronic device.

The light emitting layer 130 may include, e.g., the aforementioned composition for an organic optoelectronic device as a phosphorescent host.

The light emitting layer may further include one or more compounds in addition to the aforementioned host.

The light emitting layer may further include a dopant. The dopant may be, e.g., a phosphorescent dopant, for example a phosphorescent dopant of red, green or blue, and may be, for example, a red phosphorescent dopant.

The composition for an organic optoelectronic device further including the dopant may be, e.g., a red light emitting composition.

The dopant is a material mixed with the compound or composition for an organic optoelectronic device in a trace amount to cause light emission, and may be a material such as a metal complex that emits light by multiple excitation into a triplet or more. The dopant may be, e.g., an inorganic, organic, or organic-inorganic compound, and one or more types thereof may be used.

Examples of the dopant may be a phosphorescent dopant and examples of the phosphorescent dopant may include an organic metal compound including Ir, Pt, Os, Ti, Zr, Hf, Eu, Tb, Tm, Fe, Co, Ni, Ru, Rh, Pd, or a combination thereof. In an implementation, the phosphorescent dopant may include, e.g., a compound represented by Chemical Formula Z.

L⁵MX² [Chemical Formula Z]

In Chemical Formula Z, M may be, e.g., a metal, and L⁵ and X² may each independently be, e.g., ligands forming a complex with M.

M may be, e.g., Ir, Pt, Os, Ti, Zr, Hf, Eu, Tb, Tm, Fe, Co, Ni, Ru, Rh, Pd, or a combination thereof and L⁵ and X² may be, e.g., a bidentate ligand.

The organic layer may further include a charge transport region in addition to the light emitting layer.

The charge transport region may be, e.g., the hole transport region 140 (see, e.g., FIG. 2).

Referring to FIG. 2, an organic light emitting diode 200 may further includes the hole transport region 140 in addition to the light emitting layer 130. The hole transport region 140 may help further increase hole injection and/or hole mobility between the anode 120 and the light emitting layer 130 and block electrons. In an implementation, the hole transport region 140 may include a hole transport layer between the anode 120 and the light emitting layer 130, and a hole transport auxiliary layer between the light emitting layer 130 and the hole transport layer, and a compound of Group A may be included in at least one of the hole transport layer and the hole transport auxiliary layer.

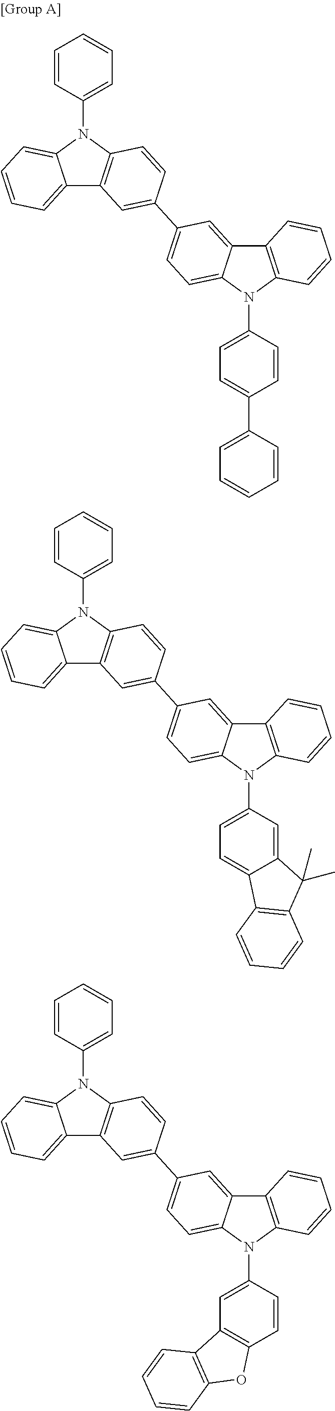 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

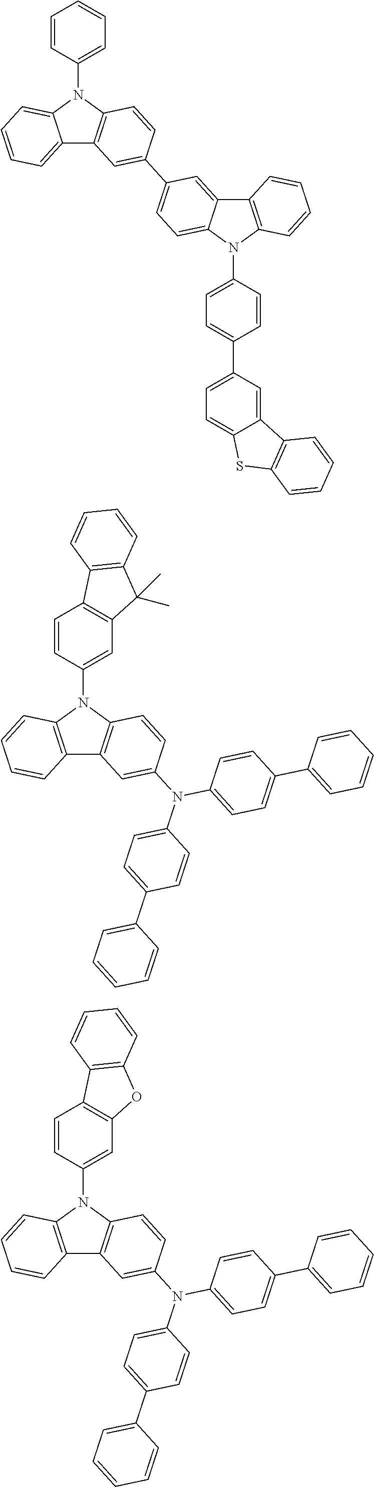

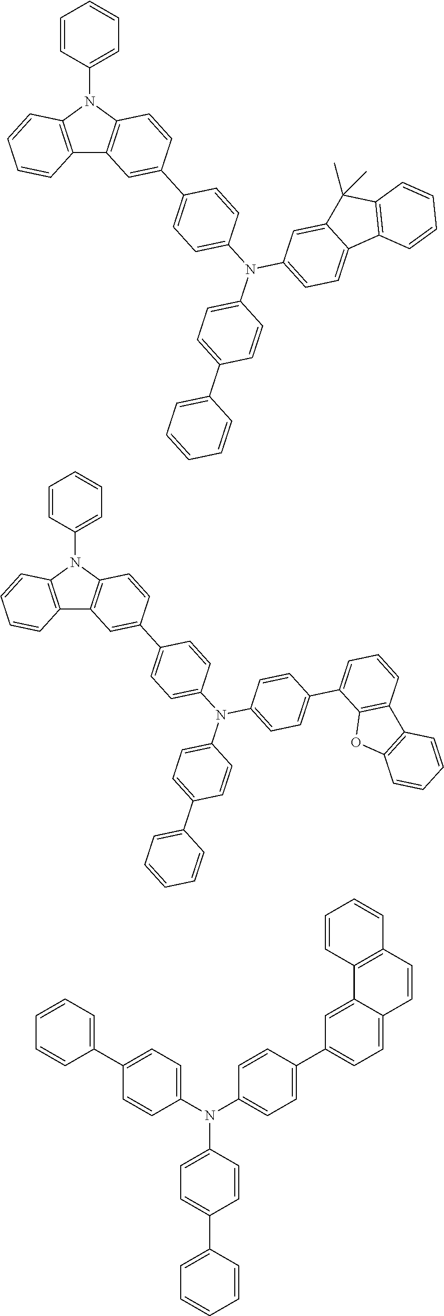 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1 ; c1ccc(-c2ccc(N(c3ccc(-c4ccccc4)cc3)c3ccc(-c4ccc5ccc6ccccc6c5c4)cc3)cc2)cc1]

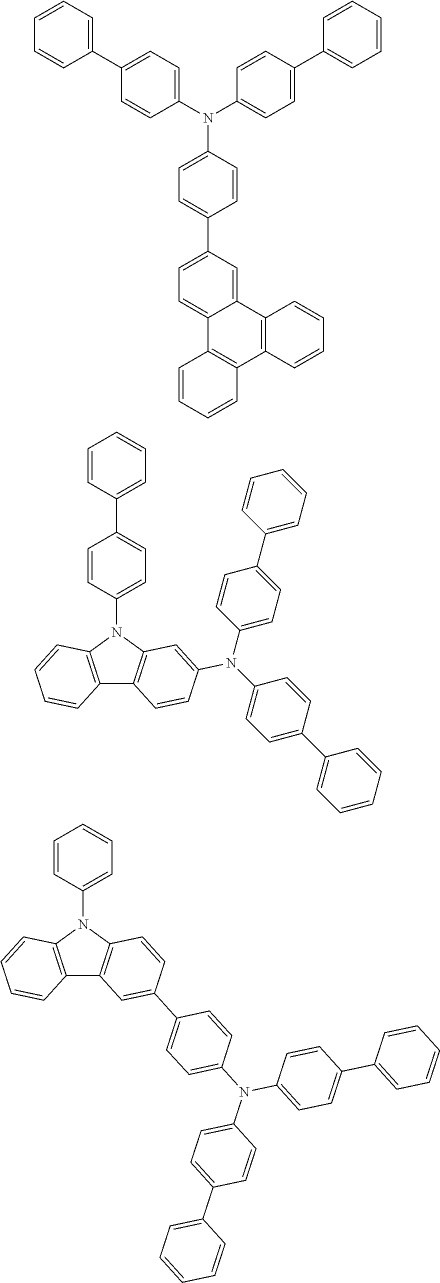 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

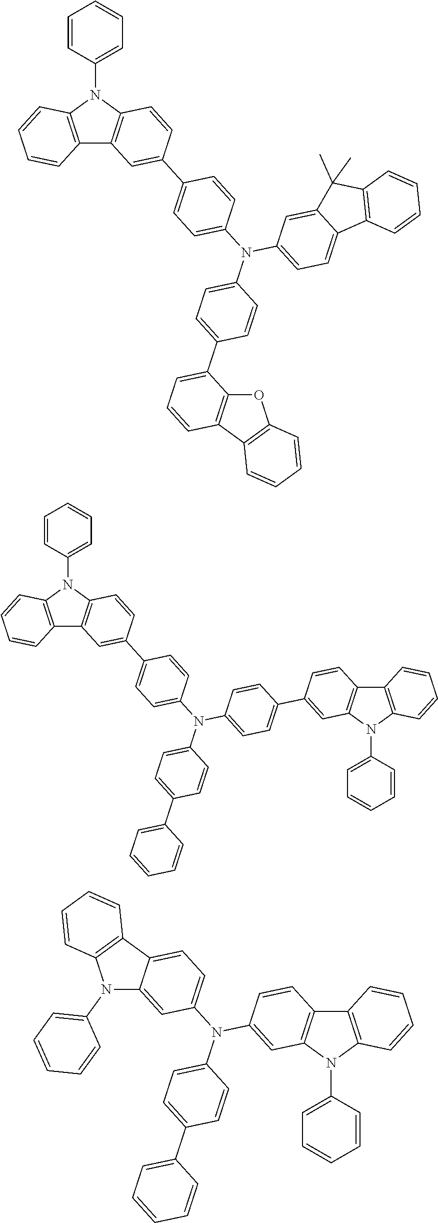

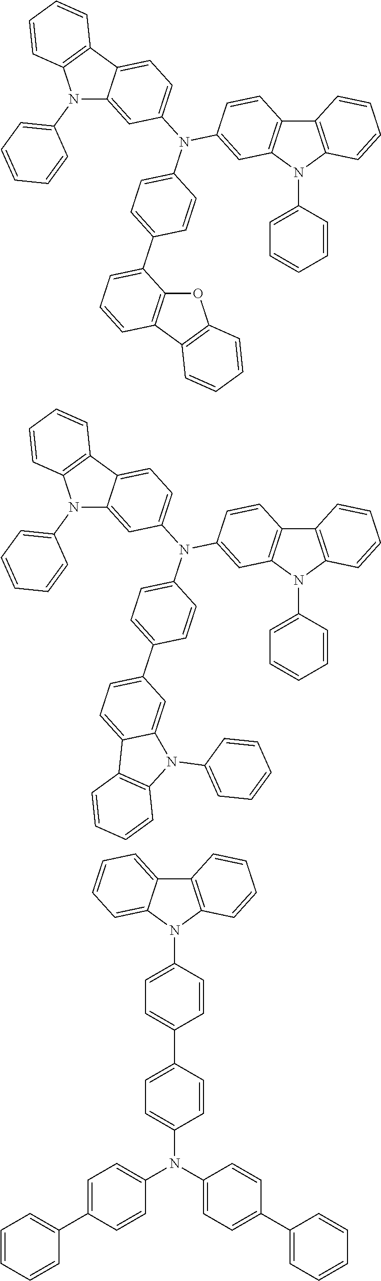

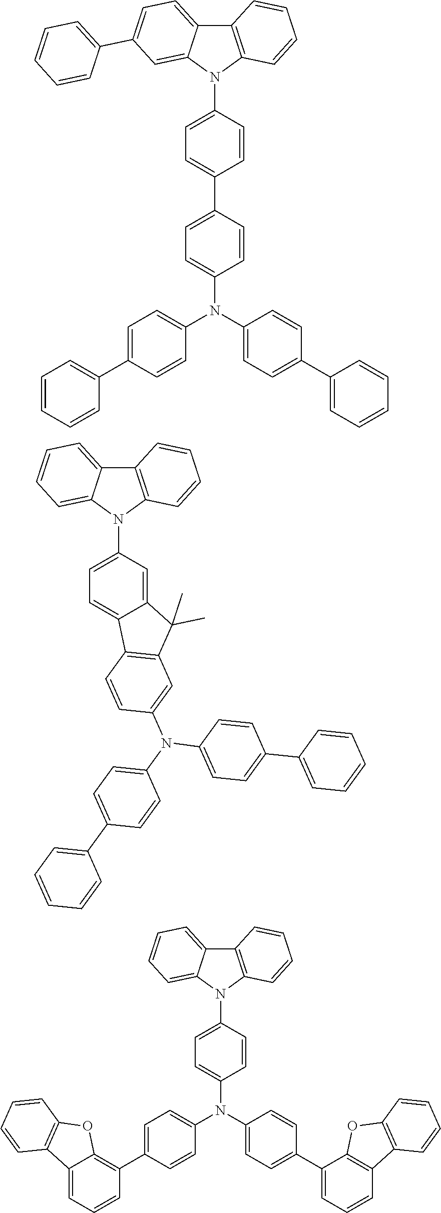

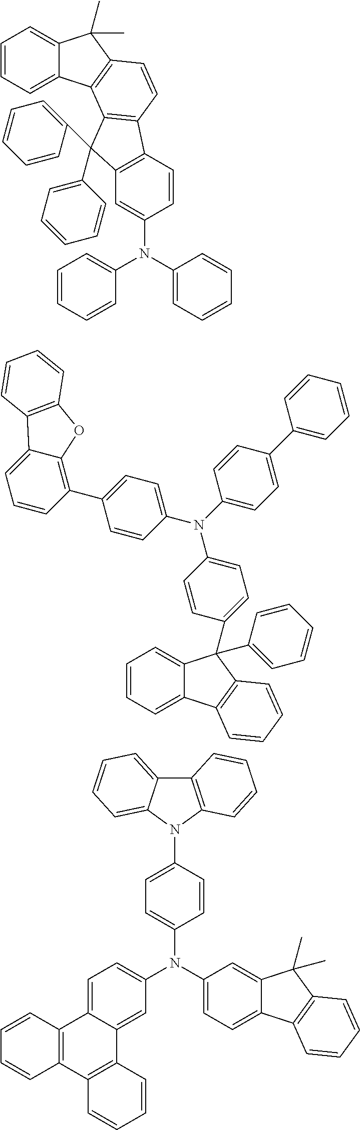

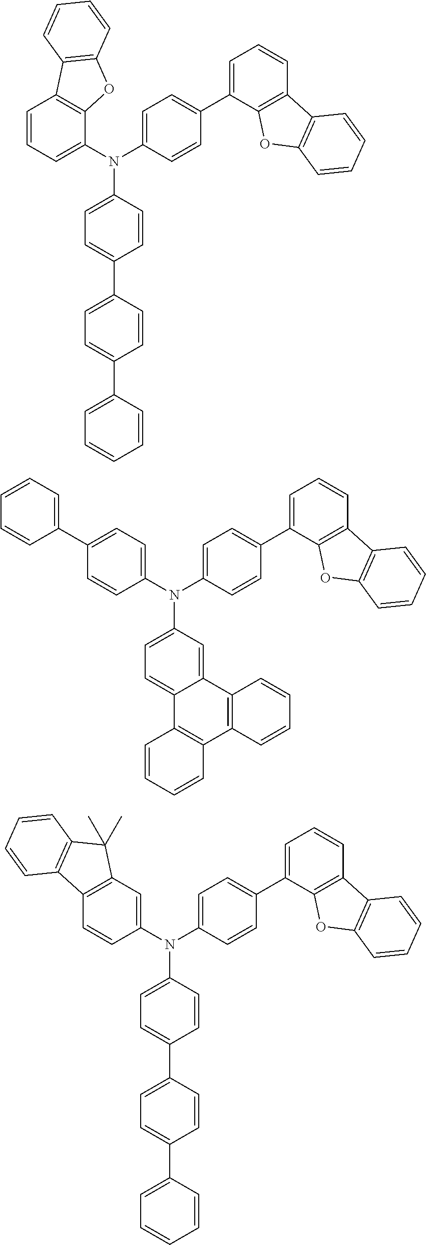

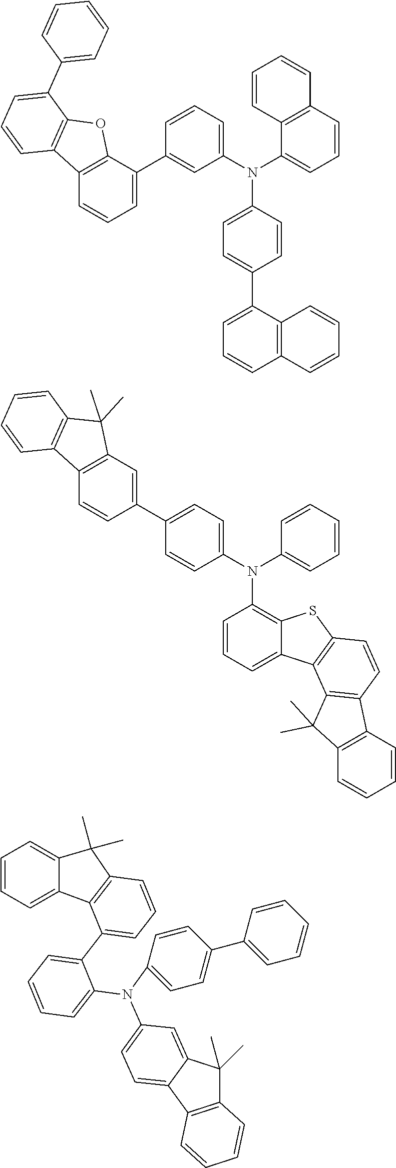 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

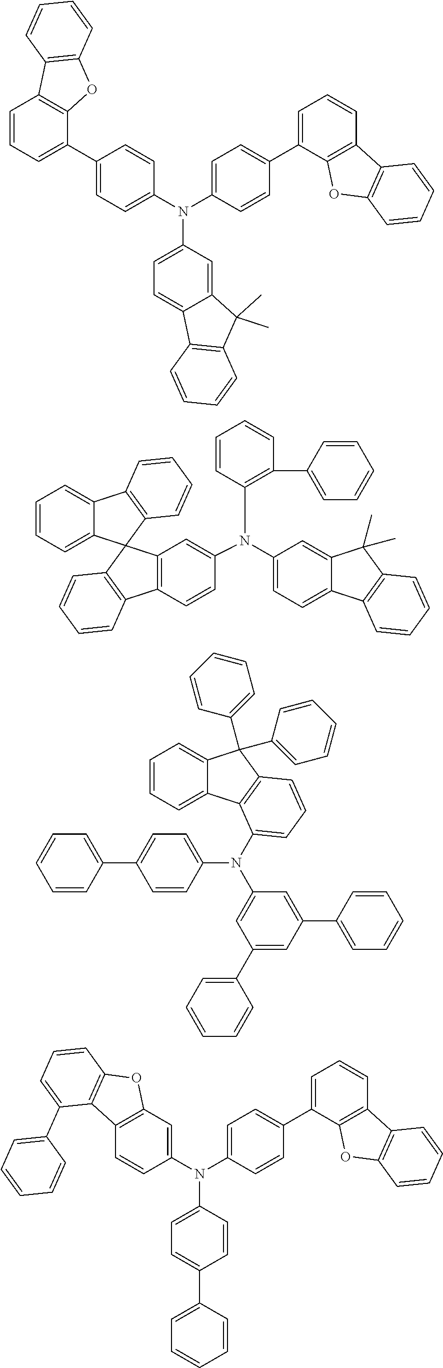

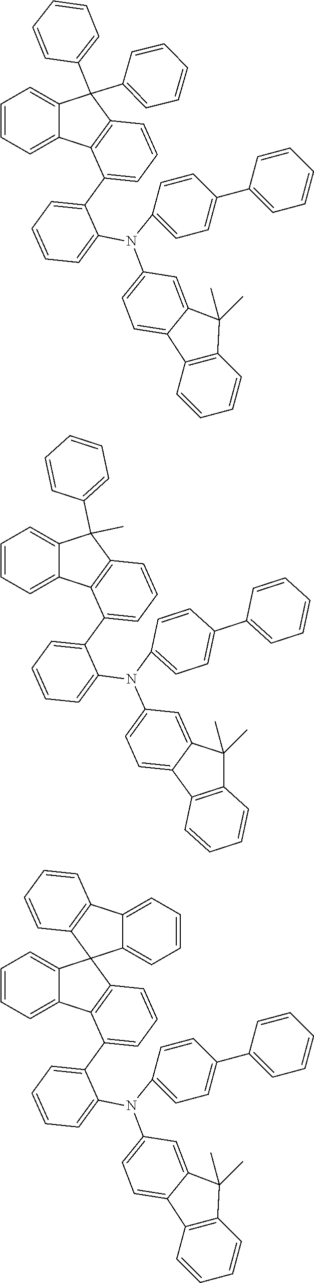 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

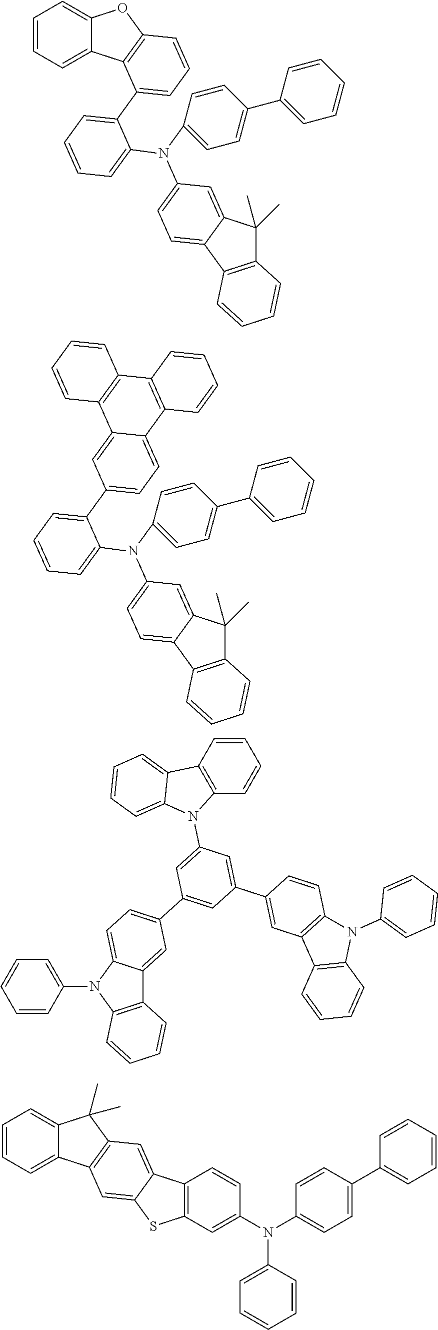

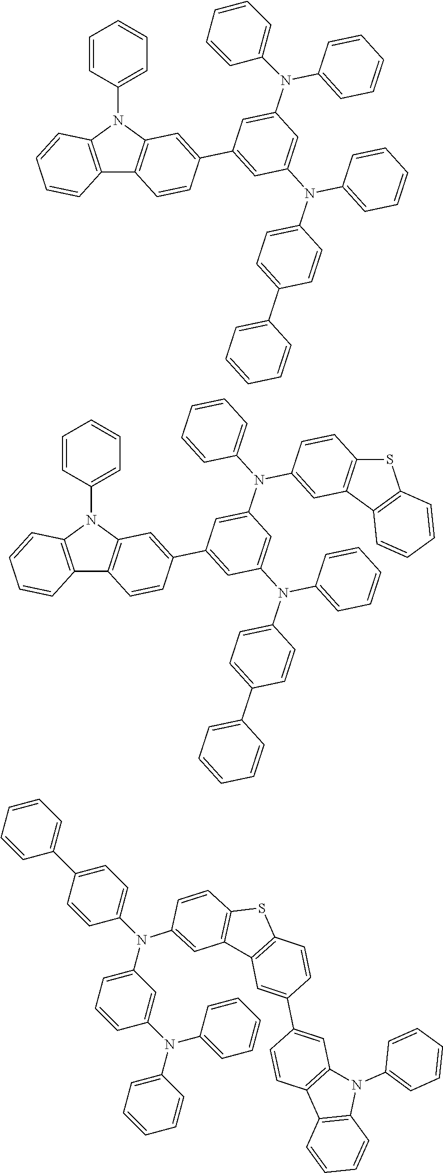

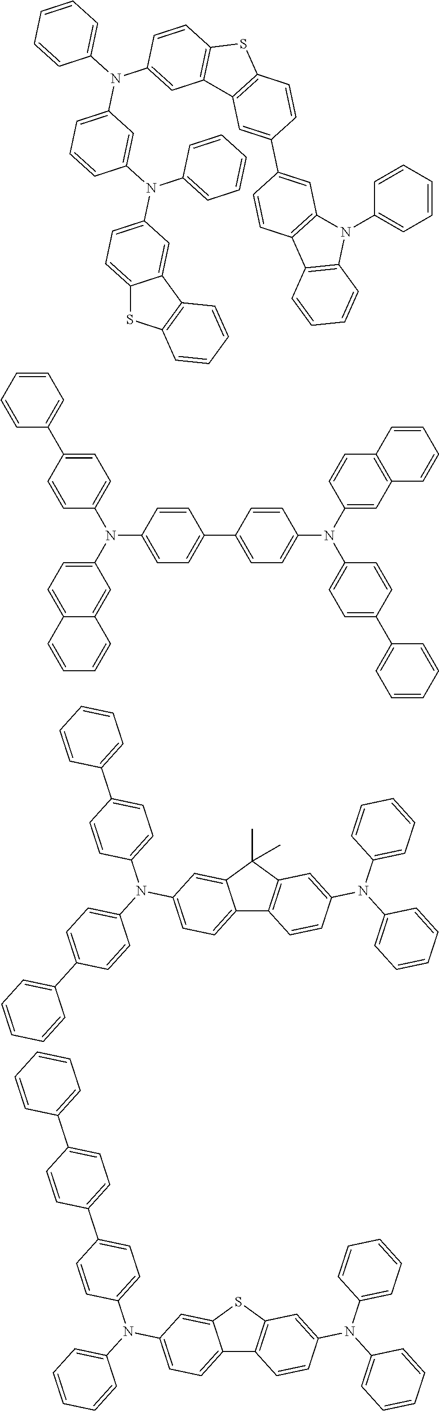

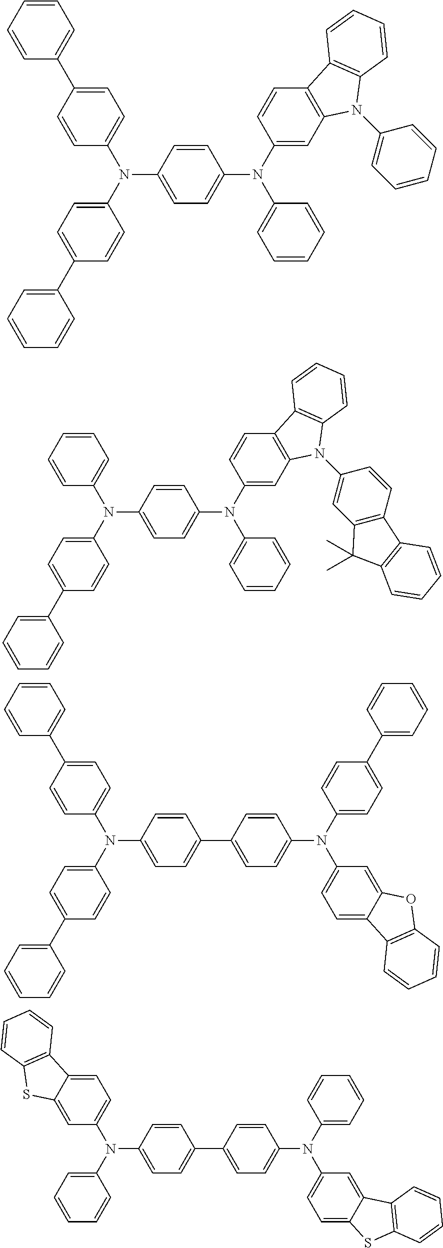

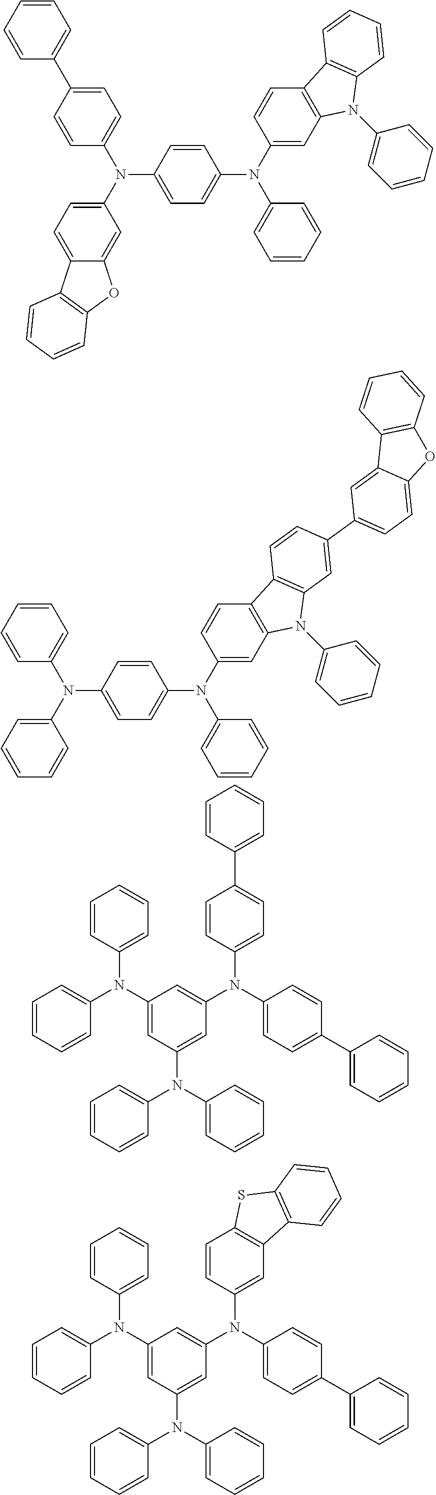

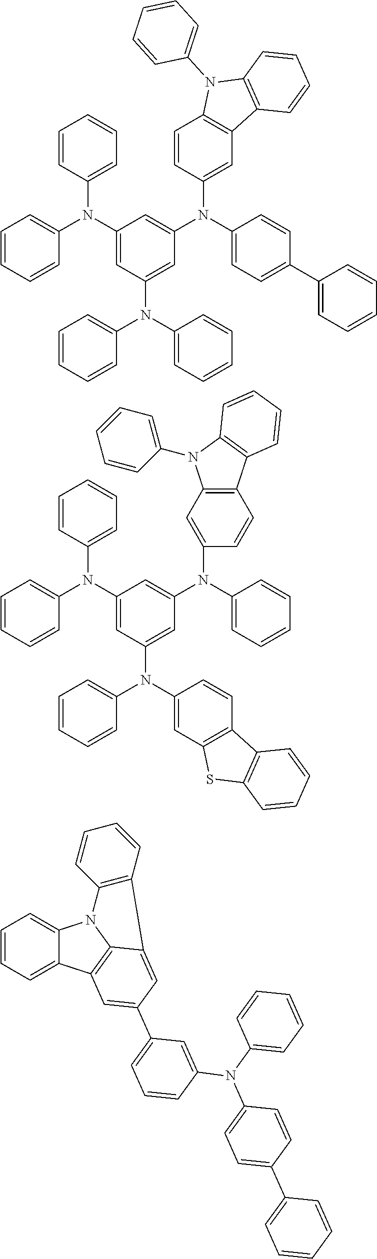

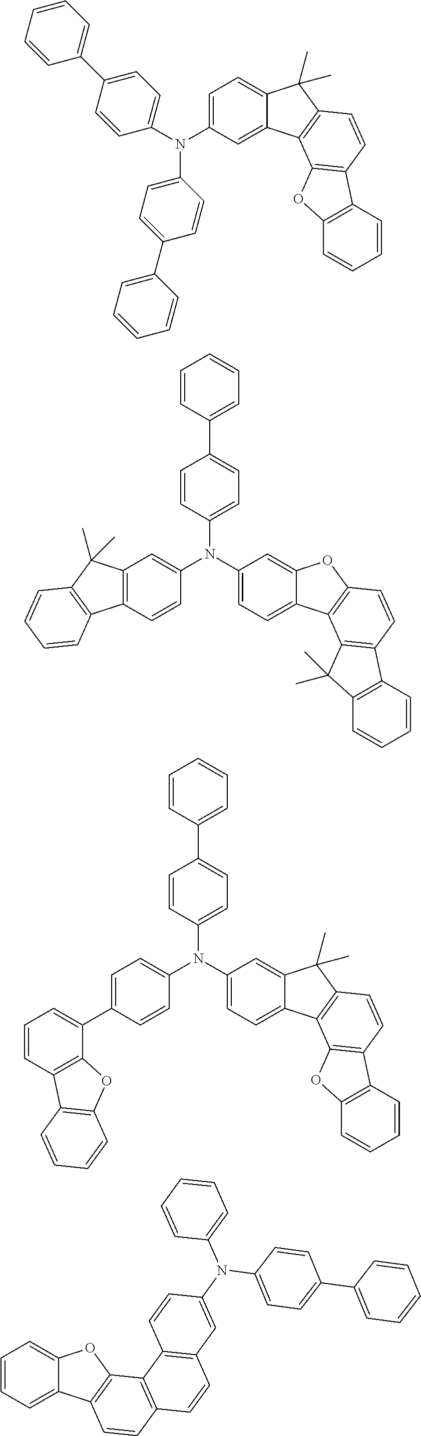

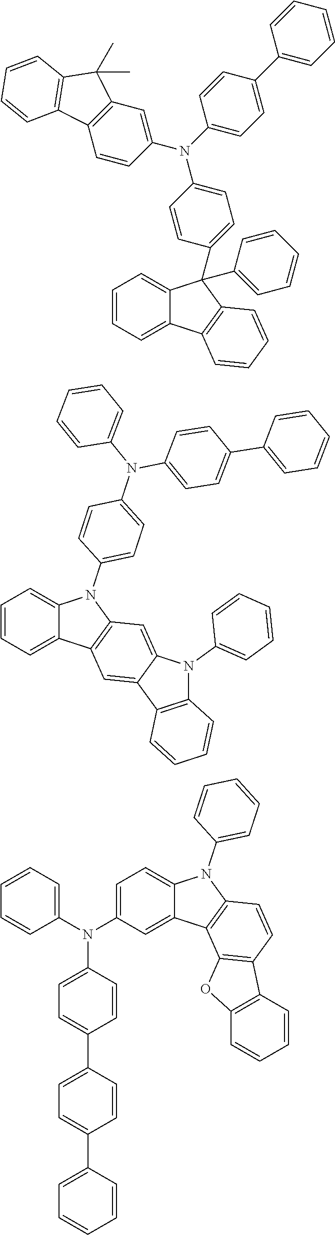

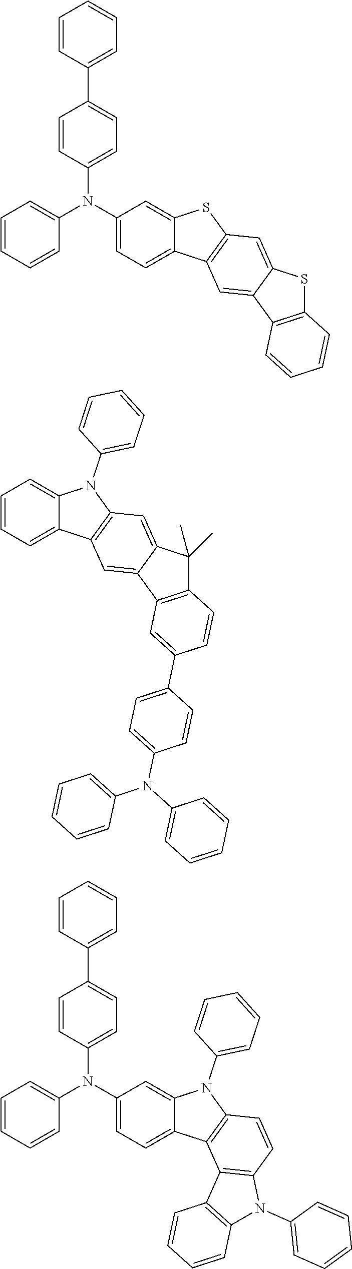

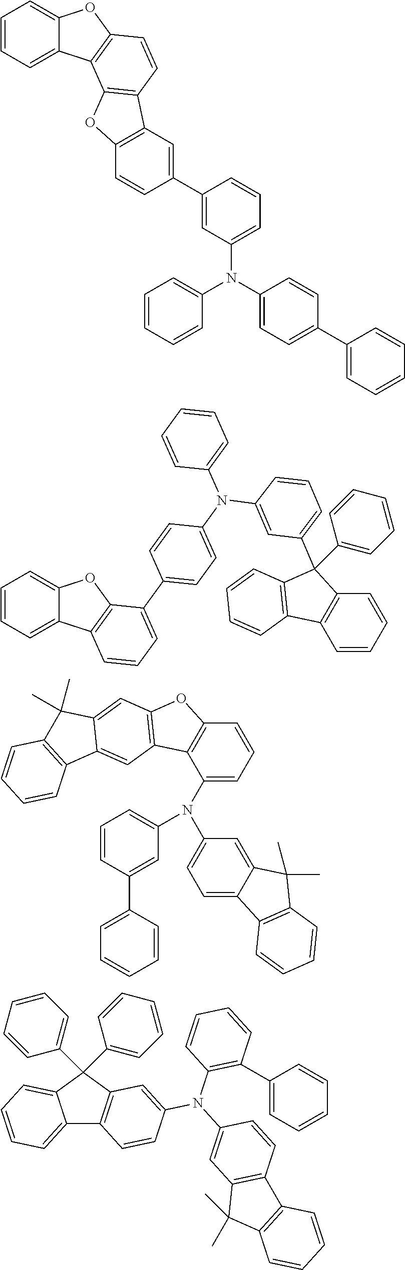

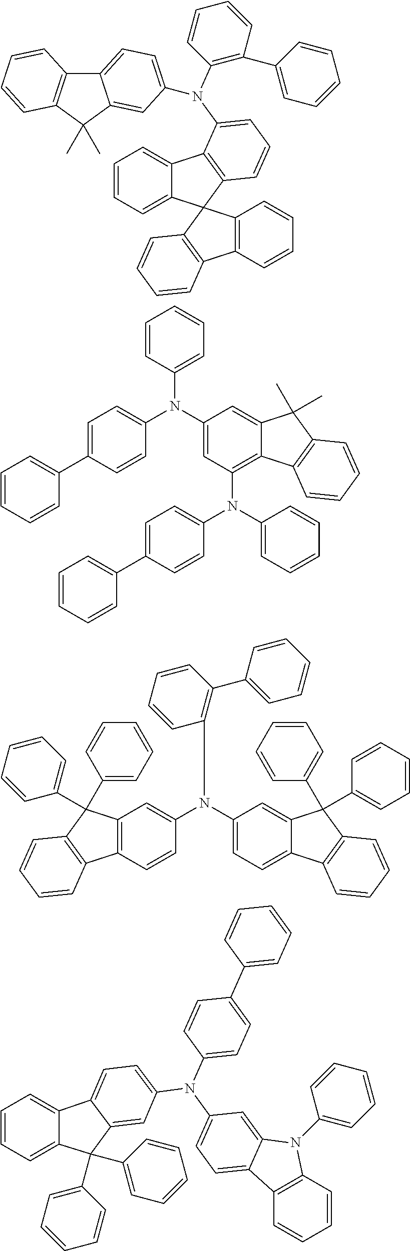

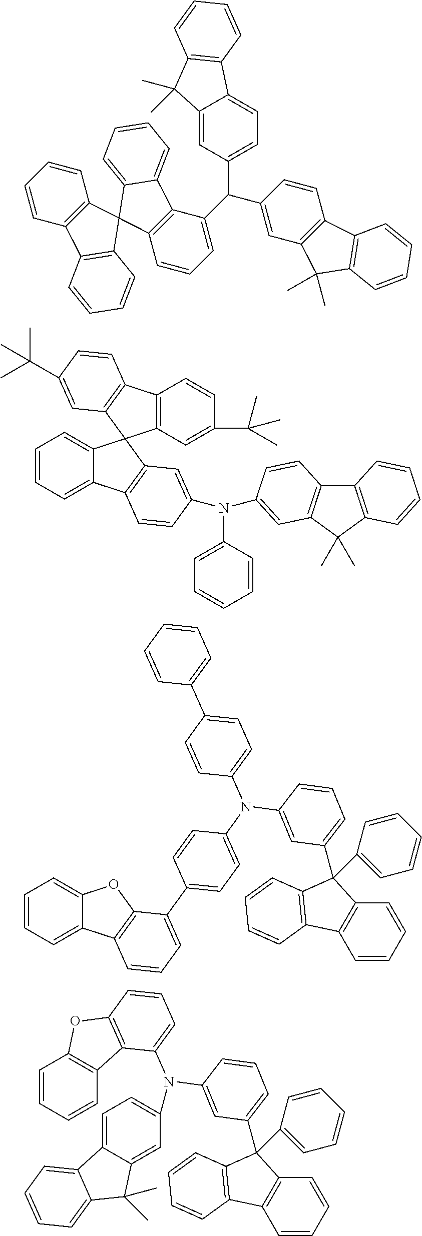

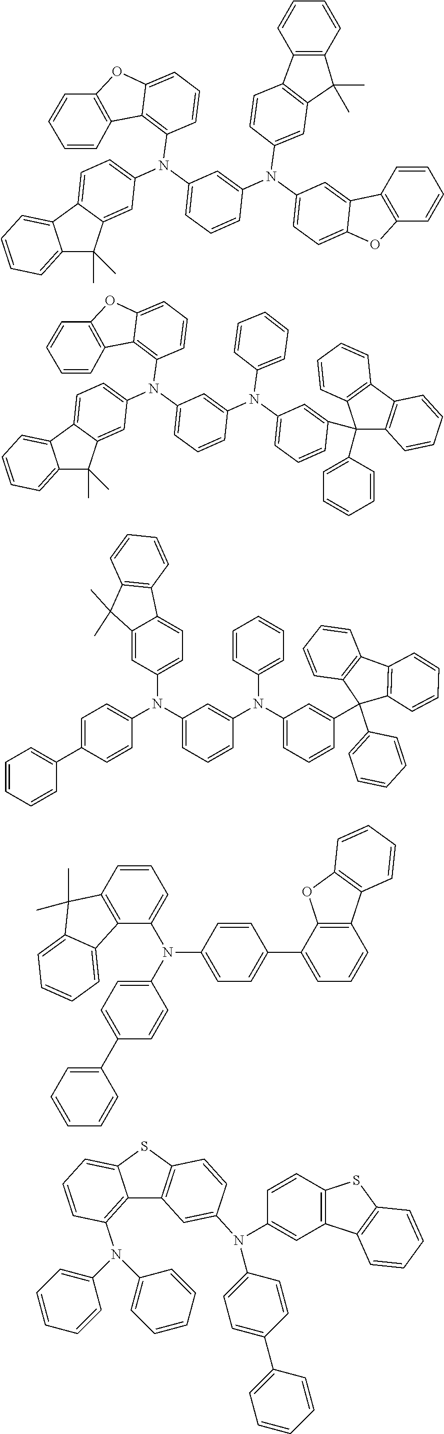

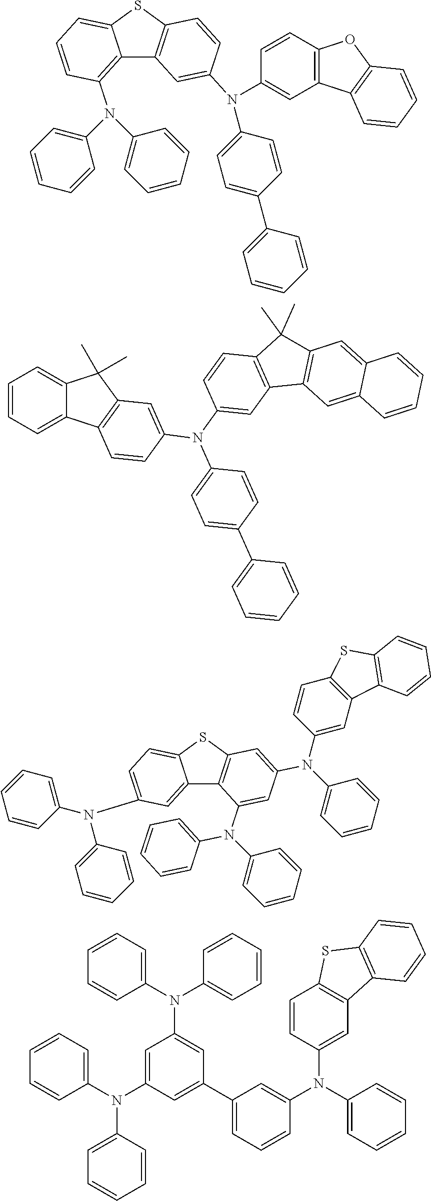

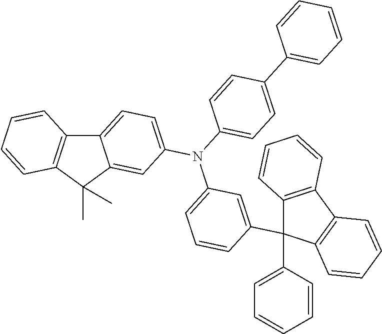 [SMILES: CC1(C)c2ccccc2-c2ccc(N(c3ccc(-c4ccccc4)cc3)c3cccc(C4(c5ccccc5)c5ccccc5-c5ccccc54)c3)cc21]

In the hole transport region, in addition to the compounds described above, other suitable compounds may also be used.

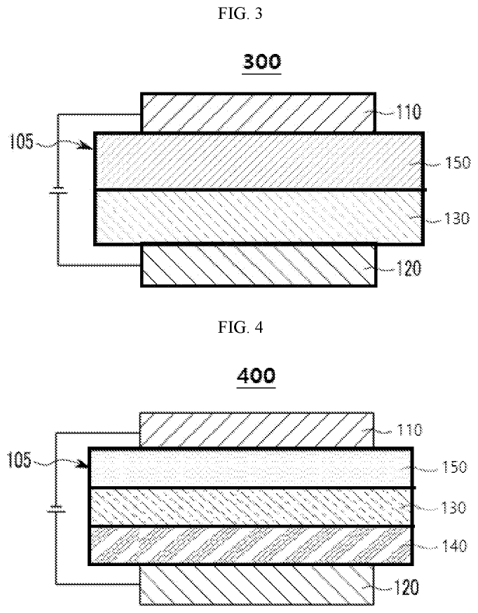

In an implementation, the charge transport region may be, e.g., the electron transport region 150 (see, e.g., FIG. 3).

Referring to FIG. 3, the organic light emitting diode 300 may further include an electron transport region 150 in addition to the light emitting layer 130. The electron transport region 150 may further increase electron injection or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer. In an implementation, a compound of Group B may be included in at least one of the electron transport layer and the electron transport auxiliary layer.

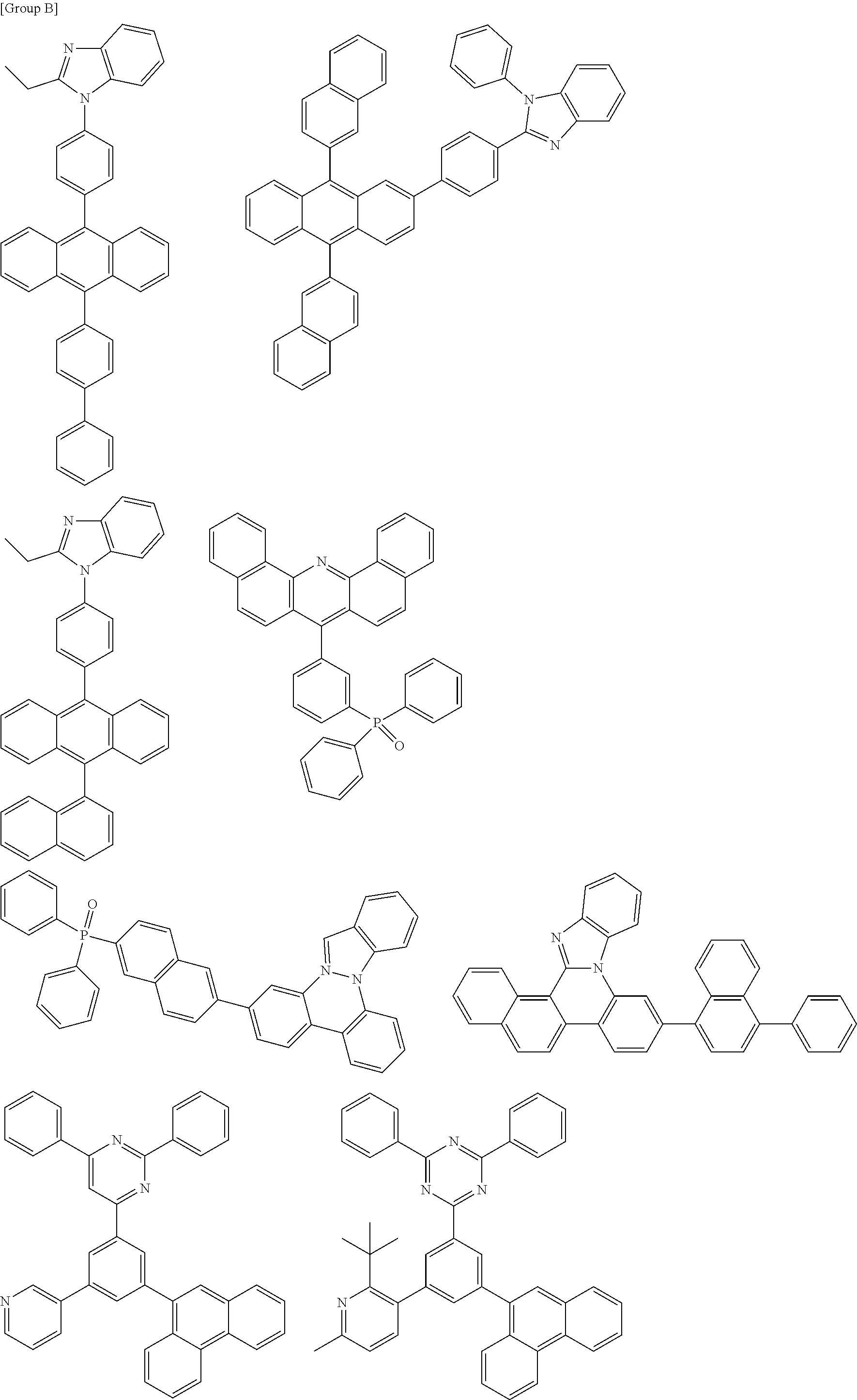

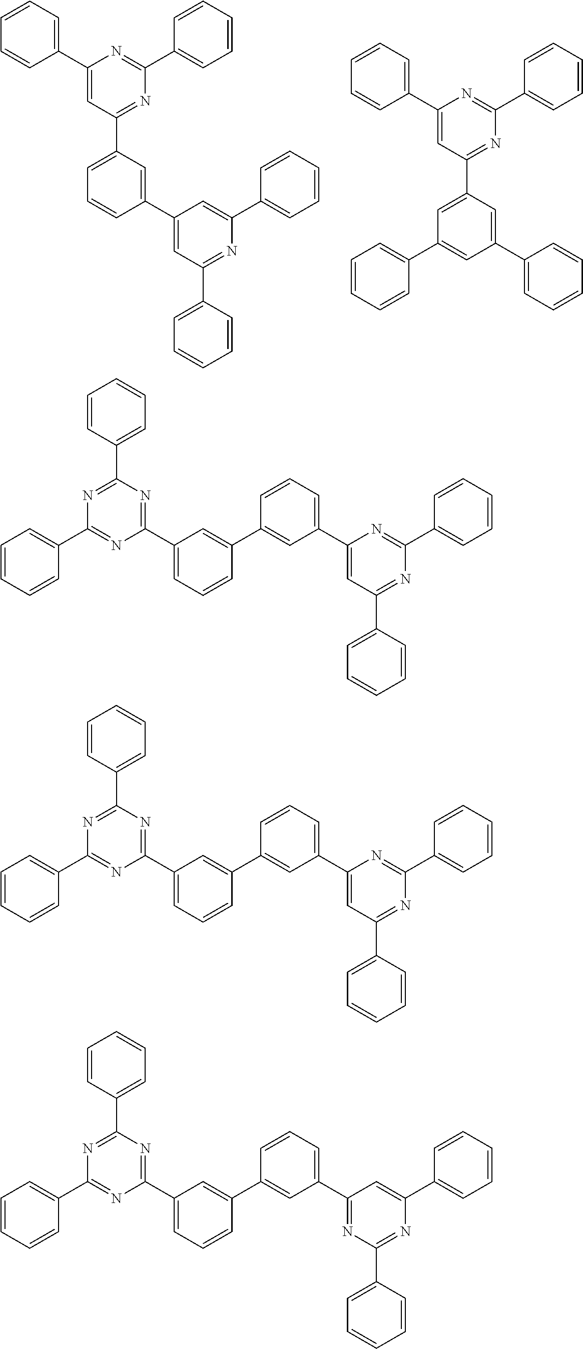 [SMILES: c1ccc(-c2cc(-c3cccc(-c4cc(-c5ccccc5)nc(-c5ccccc5)n4)c3)cc(-c3ccccc3)n2)cc1 ; c1ccc(-c2cc(-c3ccccc3)cc(-c3cc(-c4ccccc4)nc(-c4ccccc4)n3)c2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

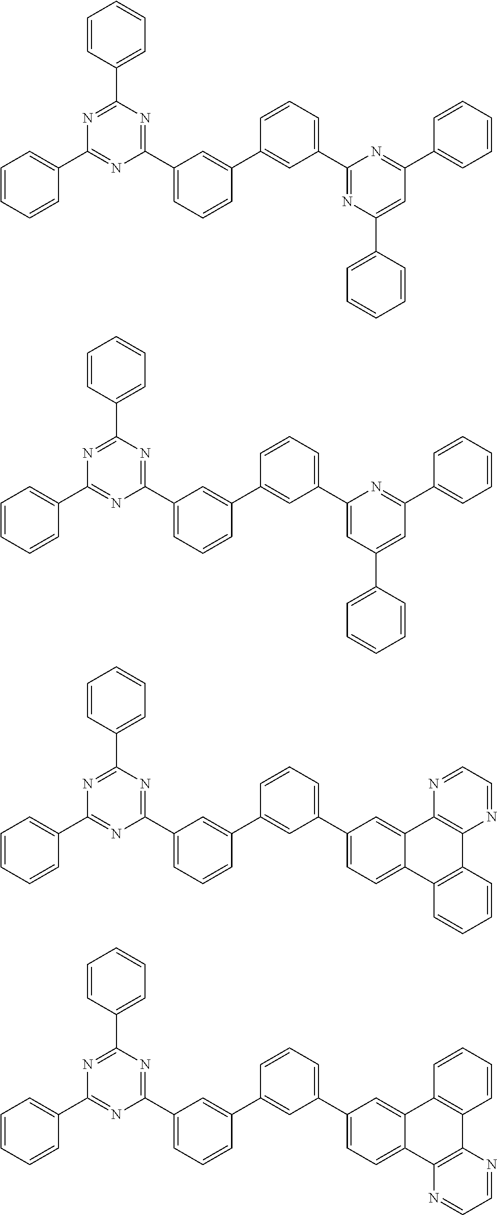 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

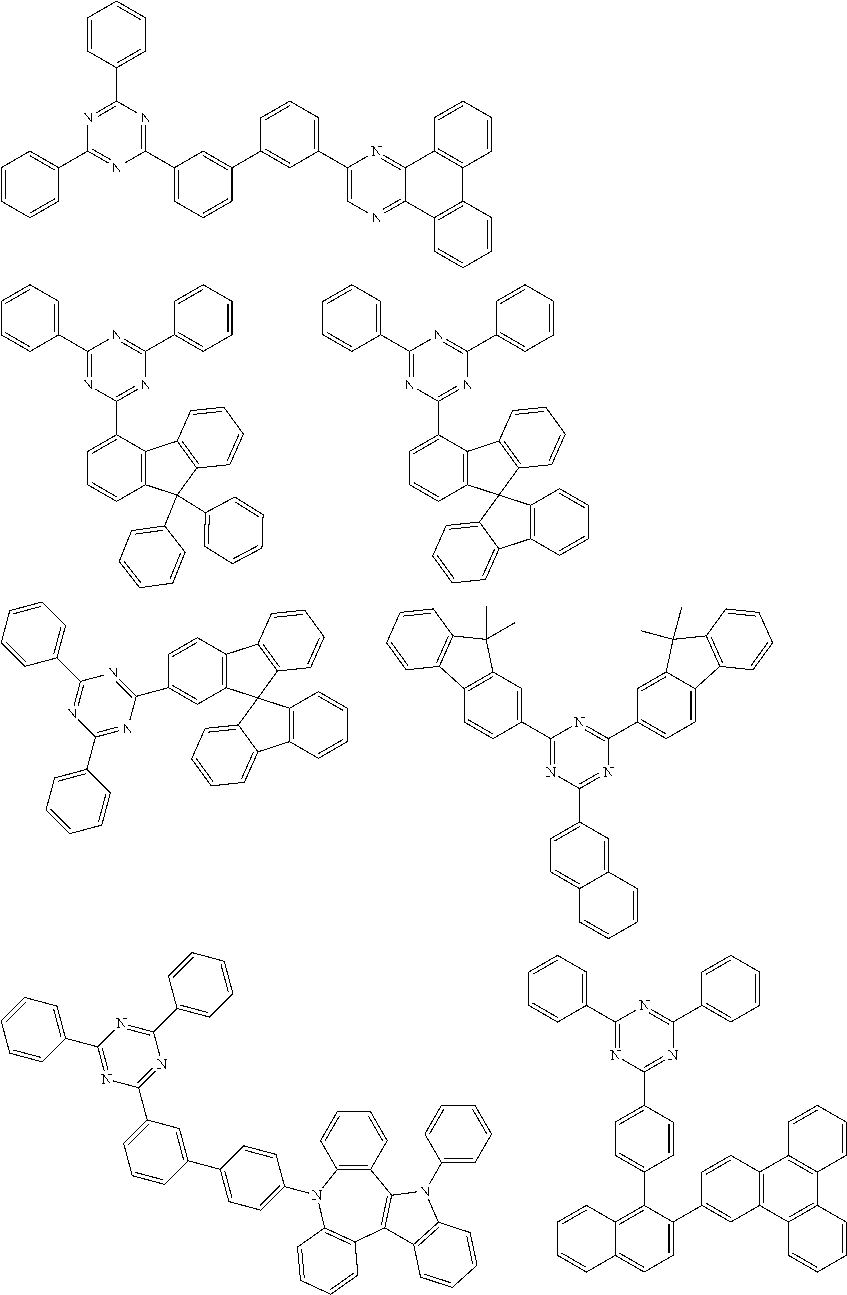

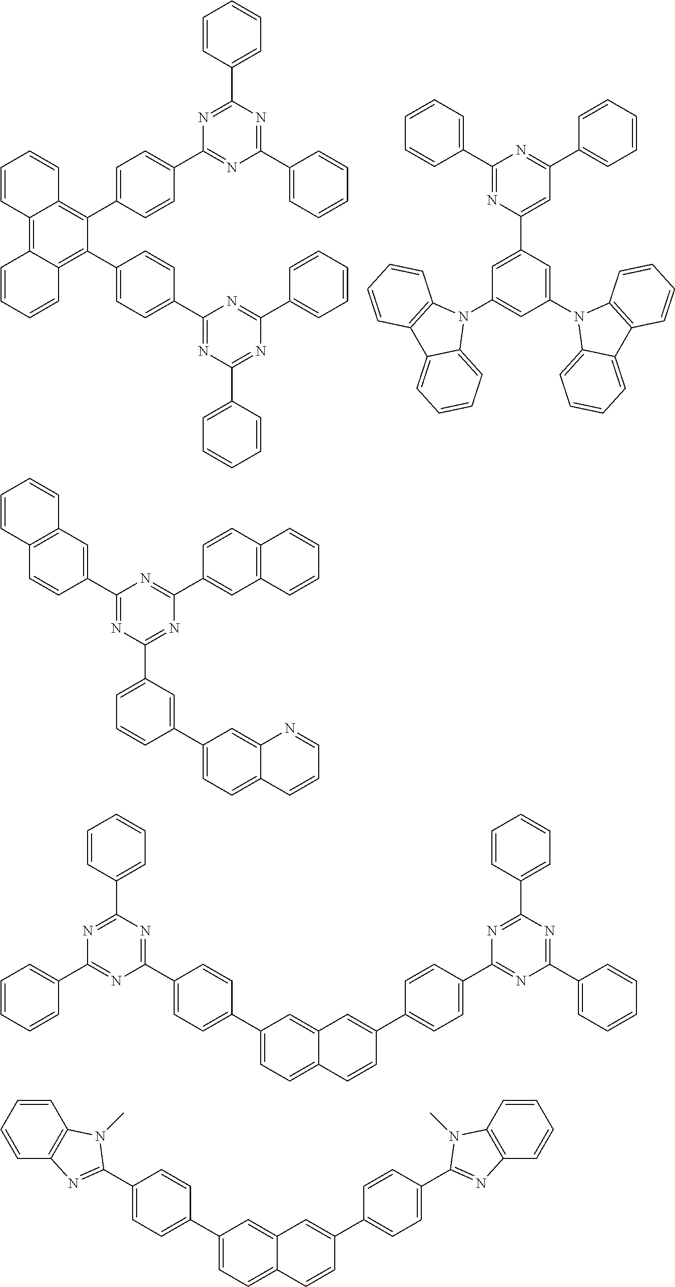

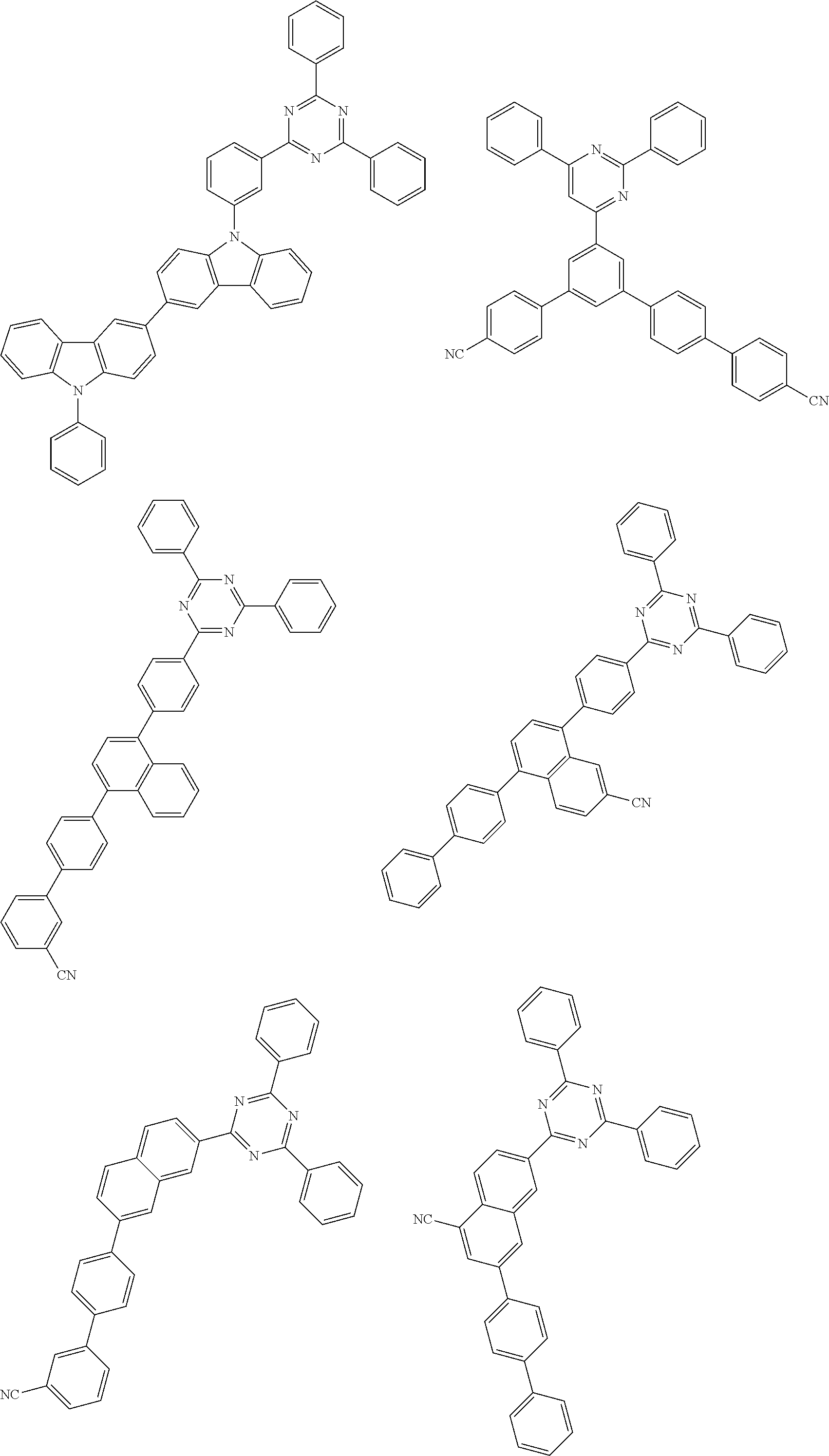

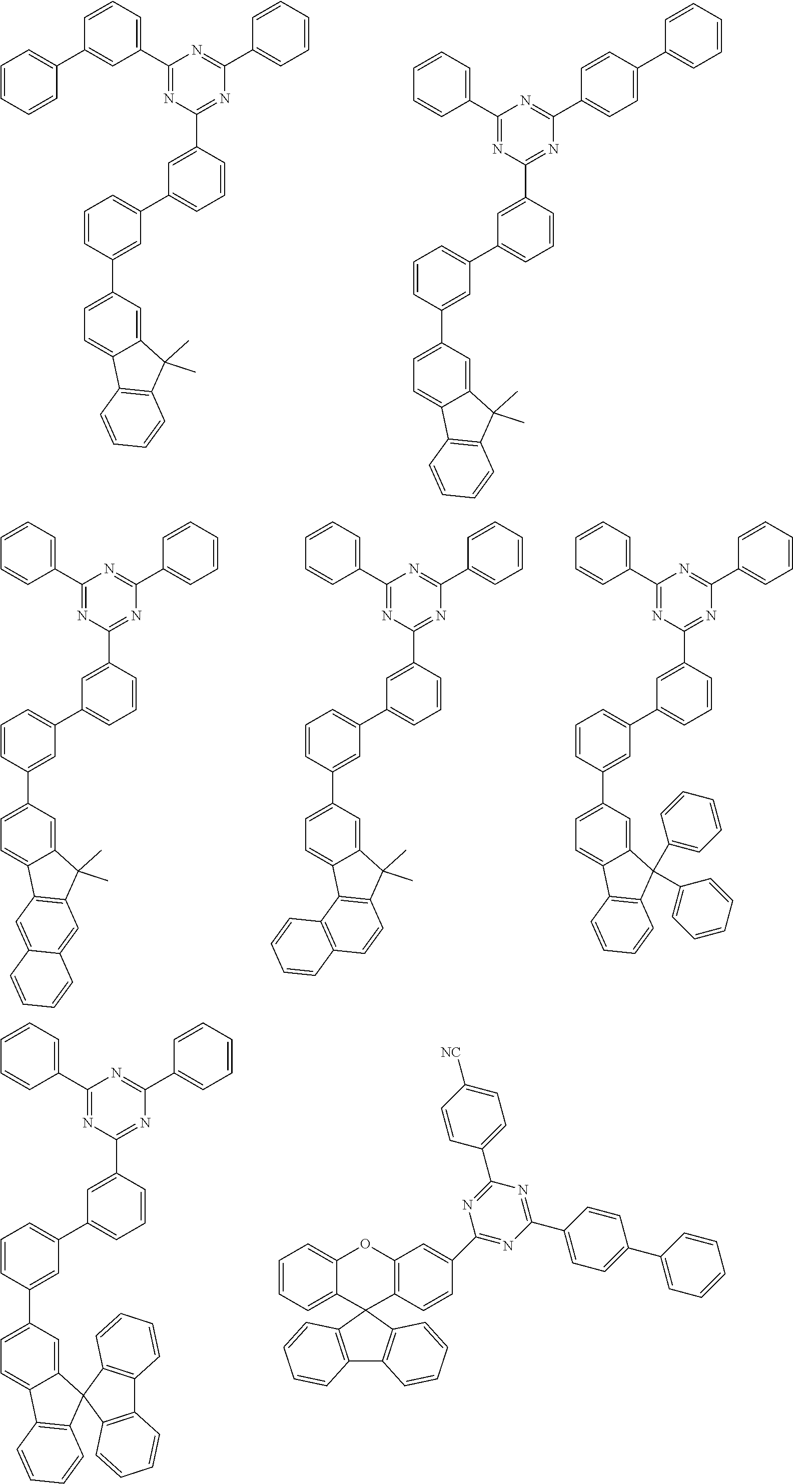

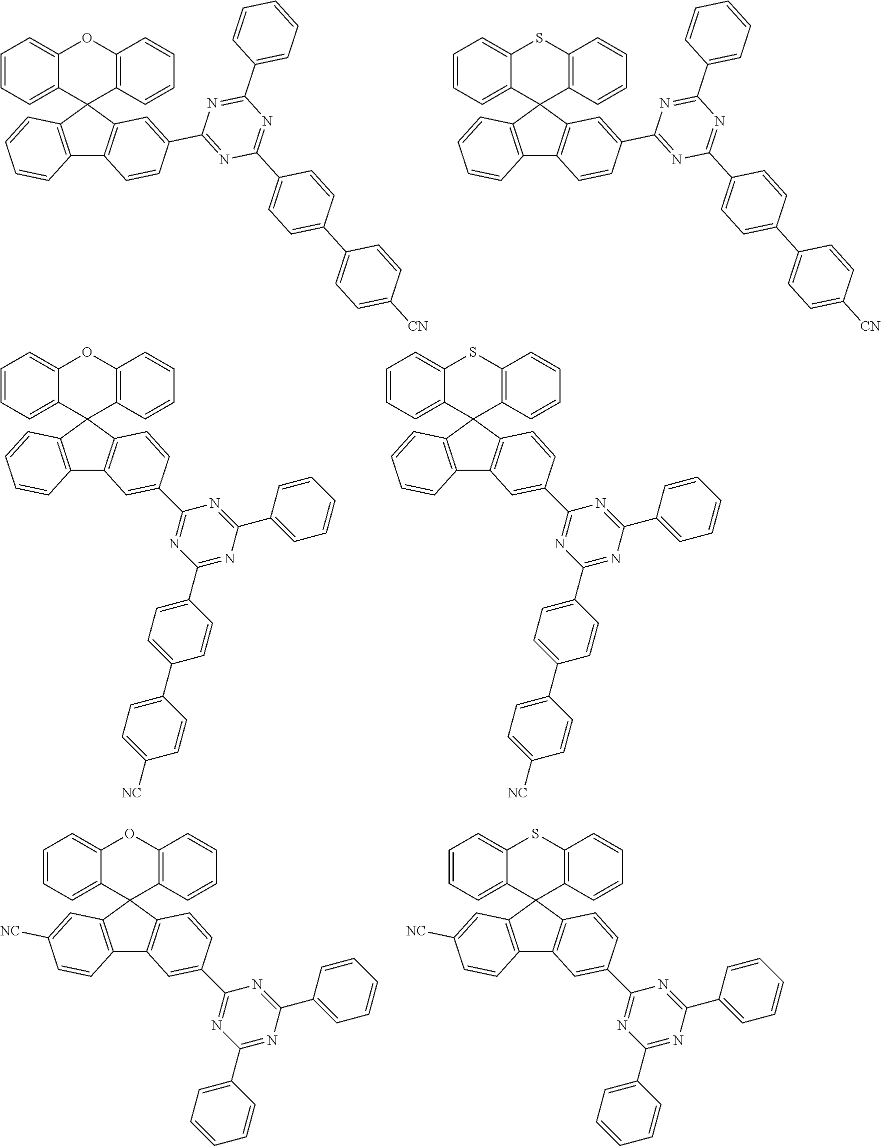

An embodiment may provide an organic light emitting diode including the light emitting layer 130 as the organic layer 105 as shown in FIG. 1.

Another embodiment may provide an organic light emitting diode including a hole transport region 140 in addition to the light emitting layer 130 as the organic layer 105, as shown in FIG. 2.

Another embodiment may provide an organic light emitting diode including an electron transport region 150 in addition to the light emitting layer 130 as the organic layer 105 as shown in FIG. 3.

Another embodiment may provide an organic light emitting diode including a hole transport region 140 and an electron transport region 150 in addition to the light emitting layer 130 as the organic layer 05, as shown in FIG. 4.

In another embodiment, an organic light emitting diode may further include an electron injection layer, a hole injection layer, or the like, in addition to the light emitting layer 130 as the organic layer 105 in each of FIGS. 1 to 4.

The organic light emitting diodes 100, 200, 300, and 400 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in the Examples and Comparative Examples were purchased from Sigma-Aldrich Co. Ltd., TCI Inc., Tokyo chemical industry or P&H tech as far as there is no particular comment or were synthesized by suitable methods.

(Preparation of Compound for Organic Optoelectronic Device)

Compounds were synthesized through the following steps.

### Synthesis of First Compound

### Synthesis Example 1: Synthesis of Compound 1-38

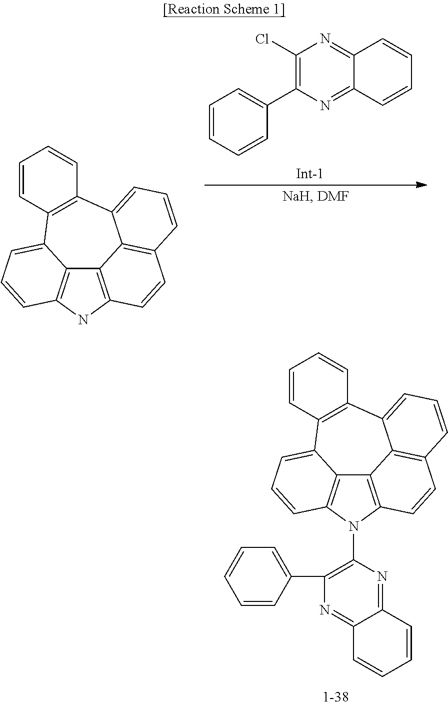 [SMILES: c1ccc2c(c1)-c1cccc3ccc4[nH]c5cccc-2c5c4c13 ; Clc1nc2ccccc2nc1-c1ccccc1 ; c1ccc(-c2nc3ccccc3nc2-n2c3cccc4c3c3c5c(cccc5ccc32)-c2ccccc2-4)cc1]

12 g (41 mmol) of 1H-3-azadibenzo[g,ij]naphtho[2,1,8-cde]azulene (CAS No. 2306215-72-3), 10.41 g (43 mmol) of Intermediate Int-1 (CAS No. 7065-92-1), dimethyl formamide (200 ml), and xylene (150 ml) were put in a round-bottomed flask, and then, stirred under a nitrogen atmosphere at ambient temperature. Subsequently, 3.3 g (82 mmol) of sodium hydride was slowly added thereto and then, stirred at ambient temperature for 12 hours. When a reaction was completed, methanol and distilled water were added to the reactant at 0° C. and then, stirred and filtered, and a solid obtained therefrom was washed with distilled water. The solid was dissolved in toluene and then, filtered through a silica gel pad, and a filtrate therefrom was concentrated under a reduced pressure and recrystallized in toluene, obtaining 15 g (Yield: 74%) of Compound 1-38.

### Synthesis Example 2: Synthesis of Compound 1-10

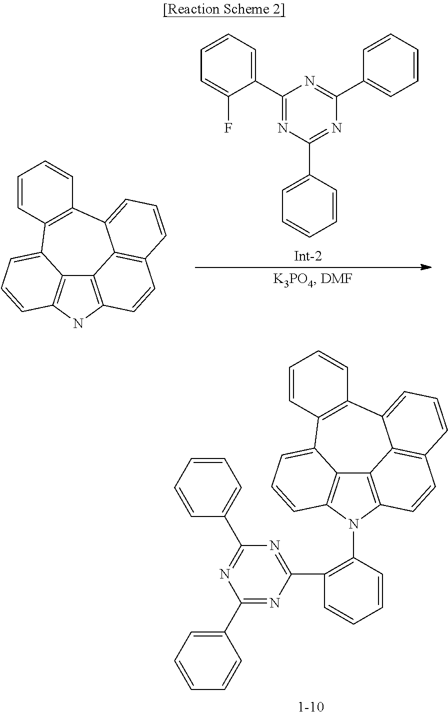 [SMILES: c1ccc2c(c1)-c1cccc3ccc4[nH]c5cccc-2c5c4c13 ; Fc1ccccc1-c1nc(-c2ccccc2)nc(-c2ccccc2)n1 ; c1ccc(-c2nc(-c3ccccc3)nc(-c3ccccc3-n3c4cccc5c4c4c6c(cccc6ccc43)-c3ccccc3-5)n2)cc1]

10 g (34 mmol) of 1H-3-azadibenzo[g,ij]naphtho[2,1,8-cde]azulene (CAS No. 2306215-72-3), 11.8 g (36 mmol) of Intermediate Int-2 (CAS No. 1818371-42-4), 14.6 g (69 mmol) of K₃PO₄, dimethyl formamide (170 ml), and xylene (170 ml) were put in a round-bottomed flask and then, stirred, while heating. When a reaction was completed, the resultant was concentrated under a reduced pressure and then, extracted with methylene chloride (MC) and distilled water. The extract was concentrated under a reduced pressure and then, recrystallized with MC and hexane, obtaining 19.1 g (Yield: 93%) of Compound 1-10.

### Synthesis of Second Compound

### Synthesis Example 3: Synthesis of Compound 2-H-3

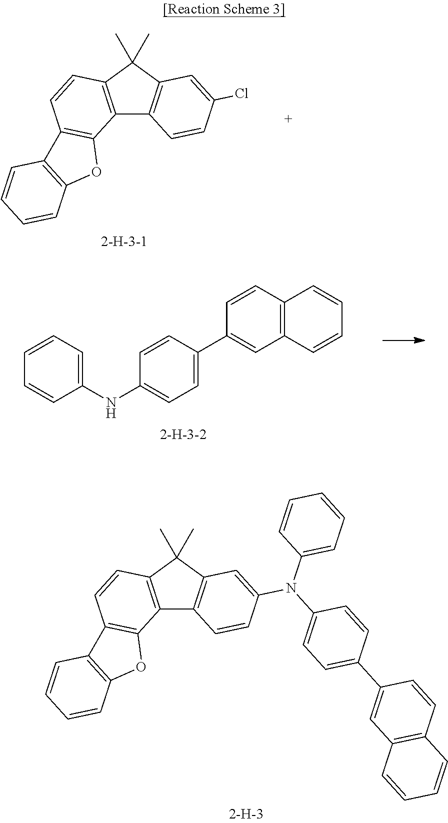 [SMILES: CC1(C)c2cc(Cl)ccc2-c2c1ccc1c2oc2ccccc21 ; c1ccc(Nc2ccc(-c3ccc4ccccc4c3)cc2)cc1 ; CC1(C)c2cc(N(c3ccccc3)c3ccc(-c4ccc5ccccc5c4)cc3)ccc2-c2c1ccc1c2oc2ccccc21]

5.0 g (15.68 mmol) of Intermediate 2-H-3-1 (CAS No. 1374677-42-5), Intermediate 2-H-3-2 (CAS No. 897671-79-3) (4.63 g, 15.68 mmol), 2.3 g (24.0 mmol) of sodium t-butoxide, and 0.1 g (0.47 mmol) of tri-tert-butylphosphine were dissolved in 200 ml of toluene, and 0.27 g (0.47 mmol) of Pd(dba)₂ was added thereto and then, stirred under reflux for 12 hours under a nitrogen atmosphere. When a reaction was completed, an organic layer extracted therefrom with toluene and distilled water was dried with anhydrous magnesium sulfate, dried, and filtered, and a filtrate therefrom was concentrated under a reduced pressure. A product therefrom was purified with normal hexane/MC (volume ratio 2:1) through silica gel column chromatography, obtaining 7.2 g (Yield: 80%) of Compound 2-H-3 as a white solid.

### Synthesis Example 4: Synthesis of Compound 2-H-2

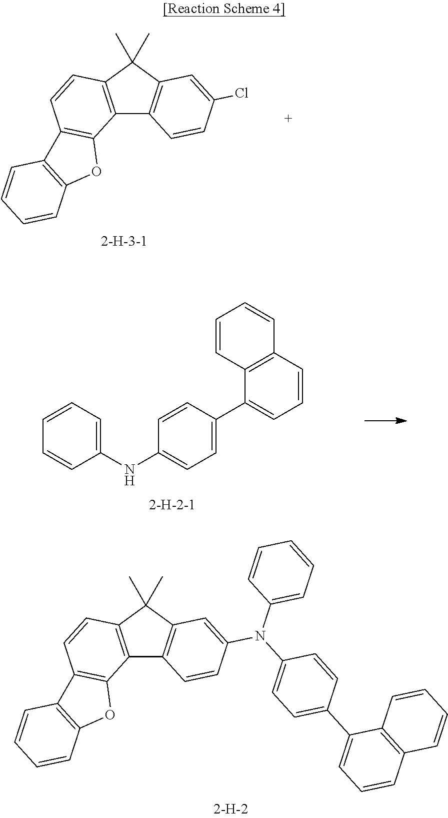 [SMILES: CC1(C)c2cc(Cl)ccc2-c2c1ccc1c2oc2ccccc21 ; c1ccc(Nc2ccc(-c3cccc4ccccc34)cc2)cc1 ; CC1(C)c2cc(N(c3ccccc3)c3ccc(-c4cccc5ccccc45)cc3)ccc2-c2c1ccc1c2oc2ccccc21]

Compound 2-H-2 was synthesized according to the same method as Synthesis Example 3 except that Intermediate 2-H-2-1 (CAS No. 897671-78-2) was used instead of Intermediate 2-H-3-2.

### Comparative Synthesis Example 1: Synthesis of Compound C-1

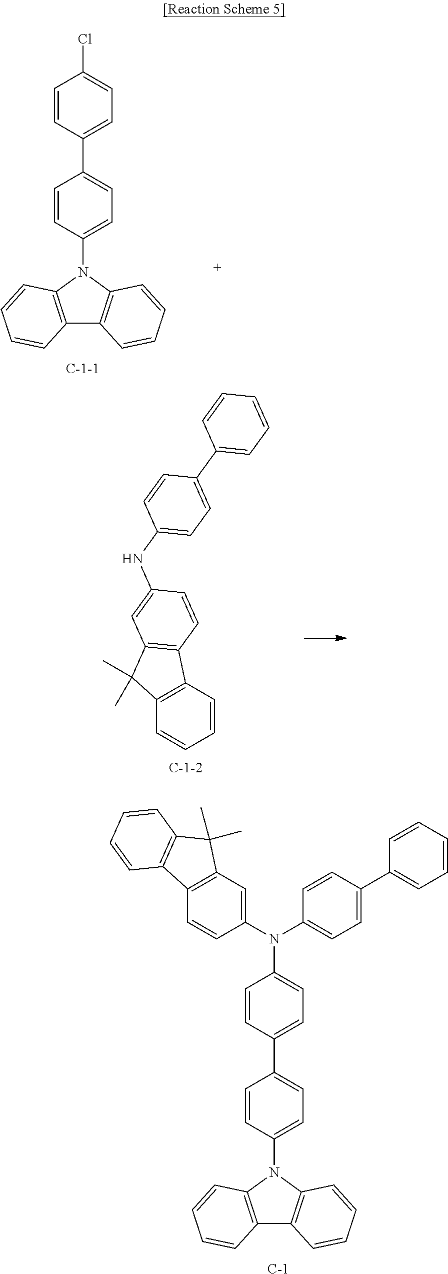

Comparative Compound C-1 was synthesized according to the same method as Synthesis Example 3 except that Intermediate C-1-1 (CAS No. 780821-30-9) and Intermediate C-1-2 (CAS No. 897671-69-1) were used instead of Intermediate 2-H-3-1 and Intermediate 2-H-3-2.

### Comparative Synthesis Example 2: Synthesis of Compound C-2

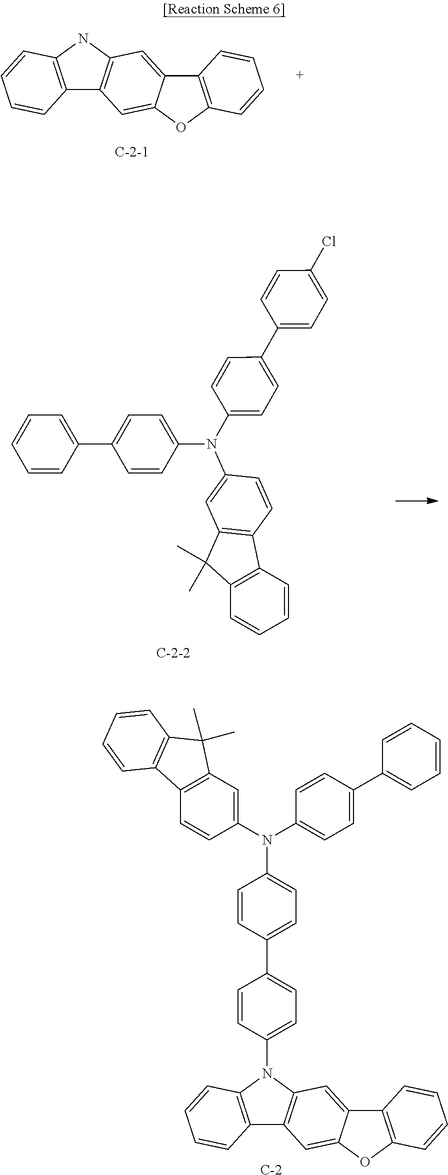

Comparative Compound C-2 was synthesized according to the same method as Synthesis Example 3 except that Intermediate C-2-1 (CAS No.: 1199350-22-5) and Intermediate C-2-2 (CAS No.: 1391737-68-0) were used instead of Intermediate 2-H-3-1 and Intermediate 2-H-3-2.

(Manufacture of Organic Light Emitting Diode)

### Example 1

A glass substrate coated with a thin film of indium tin oxide (ITO) was washed with distilled water and ultrasonic waves. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 1% NDP-9 (commercially available from Novaled) was vacuum-deposited on an ITO substrate to form a 1,400 Å-thick hole transport layer, and Compound B was deposited on the hole transport layer to form a 600 Å-thick hole transport auxiliary layer. On the hole transport auxiliary layer, Compound 1-38 obtained in Synthesis Example 1 and Compound 2-H-2 obtained in Synthesis Example 4 were simultaneously deposited (as a host), and doped with 2 wt % of [Ir(piq)₂acac] as a dopant to form a 400 Å-thick light emitting layer by vacuum deposition. In the light emitting layer, Compound 1-38 and Compound 2-H-2 were used in a weight ratio of 50:50. Then, Compound C was deposited on the light emitting layer to form a 50 Å-thick electron transport auxiliary layer, and Compound D and Liq were simultaneously vacuum-deposited at a weight ratio of 1:1 to form a 300 Å-thick electron transport layer. 15 Å of LiQ and 1,200 Å of Al were sequentially vacuum-deposited on the electron transport layer to form a cathode, manufacturing an organic light emitting diode.

ITO/Compound A (1% NDP-9 doping, 1,400 Å)/Compound B (600 Å) /EML [Compound 1-38:Compound 2-H-2:[Ir(piq)₂acac] (2 wt %)] (400 Å)/Compound C (50 Å)/Compound D:Liq (300 Å)/LiQ (15 Å)/Al (1,200 Å).

Compound A: N-(biphenyl-4-yl)-9,9-dimethyl-N-(4-(9-phenyl-9H-carbazol-3-yl)phenyl)-9H-fluoren-2-amine

Compound B: N,N-di([1,1′-biphenyl]-4-yl)-7,7-dimethyl-7H-fluoreno[4,3-b]benzofuran-10-amine

Compound C: 2-(3-(3-(9,9-dimethyl-9H-fluoren-2-yl)phenyl)phenyl)-4,6-diphenyl-1,3,5-triazine

Compound D: 8-(4-(4,6-di(naphthalen-2-yl)-1,3,5-triazin-2-yl)phenyl)quinoline

### Example 2

An organic light emitting diode according to Example 2 was manufactured according to the same method as Example 1 except that Compound 1-38 and Compound 2-H-2 were mixed in a weight ratio of 60:40.

### Example 3

An organic light emitting diode according to Example 3 was manufactured according to the same method as Example 1 except that Compound 1-10 and Compound 2-H-3 were used instead of Compound 1-38 and Compound 2-H-2.

### Example 4

An organic light emitting diode according to Example 4 was manufactured according to the same method as Example 1 except that Compound 1-10 and Compound 2-H-3 were mixed in a weight ratio of 60:40.

### Comparative Example 1

An organic light emitting diode according to Comparative Example 1 was manufactured according to the same method as Example 1 except that Compound 2-H-2 was not used.

### Comparative Example 2

An organic light emitting diode according to Comparative Example 2 was manufactured according to the same method as Example 1 except that Compound C-1 was used instead of Compound 2-H-2.

### Comparative Example 3

An organic light emitting diode according to Comparative Example 3 was manufactured according to the same method as Example 1 except that Compound C-2 was used instead of Compound 2-H-2.

Evaluation: Confirmation of Life-Span Increase Effect

The life-span characteristics of the organic light emitting diodes according to Examples 1 to 4 and Comparative Examples 1 to 3 were evaluated. Specific measurement methods are as follows, and the results are shown in Table 1.

(1) Measurement of Life-Span

T97 life-spans of the organic light emitting diodes according to Examples 1 to 4 and Comparative Examples 1 to 3 were measured as a time when their luminance decreased down to 97% relative to the initial luminance (cd/m²) after emitting light with 9,000 cd/m² as the initial luminance (cd/m²) and measuring their luminance decrease depending on a time with a Polanonix life-span measurement system.

(2) Calculation of T97 Life-Span Ratio (%)

In Table 1, T97 life-span was evaluated based on the T97 life-span of Comparative Example 1.

TABLE 1

| | | | T97 life-span |
|---|---|---|---|
| | First host | Second host | ratio (%) |
| Example 1 | 1-38 | 2-H-2 | 200 |
| Example 2 | 1-38 | 2-H-2 | 209 |
| Example 3 | 1-10 | 2-H-3 | 206 |
| Example 4 | 1-10 | 2-H-3 | 228 |
| Comparative Example 1 | 1-38 | — | 100 |
| Comparative Example 2 | 1-38 | C-1 | — |
| Comparative Example 3 | 1-38 | C-2 | — |

Referring to Table 1, the organic light emitting diodes according to Examples 1 to 4 exhibited significantly improved life-span characteristics, compared with the organic light emitting diodes of Comparative Examples 1 to 3.

Particularly, the organic light emitting diodes according to Comparative Examples 2 and 3 exhibited too significantly deteriorated characteristics to measure a life-span.

One or more embodiments may provide a composition for an organic optoelectronic device capable of implement an organic optoelectronic device having high efficiency and a long life-span.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A composition for an organic optoelectronic device, the composition comprising:
a first compound represented by Chemical Formula 1, and
a second compound represented by Chemical Formula 2, 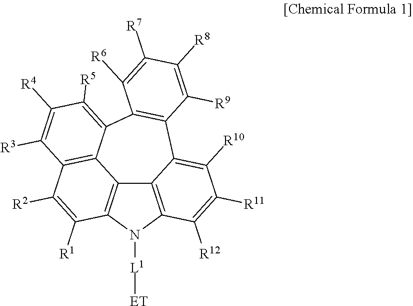
wherein, in Chemical Formula 1,
R¹ to R¹² are each independently hydrogen, deuterium, a cyano group, halogen, a substituted or unsubstituted C1 to C30 alkyl group, or a substituted or unsubstituted C6 to C30 aryl group,
L¹ is a single bond or a substituted or unsubstituted C6 to C30 arylene group, and
ET is a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzofuranpyrimidinyl group, or a substituted or unsubstituted benzothiophenepyrimidinyl group; 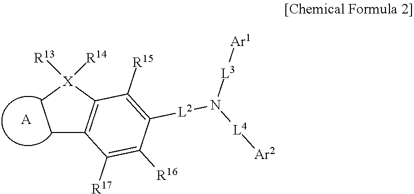
wherein, in Chemical Formula 2,
X is C or Si,
R¹³ and R¹⁴ are each independently a substituted or unsubstituted C1 to C30 alkyl group or a substituted or unsubstituted C6 to C30 aryl group,
R¹⁵ to R¹⁷ are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
L² to L⁴ are each independently a single bond or a substituted or unsubstituted C6 to C30 arylene group,
Ar¹ and Ar² are each independently a substituted or unsubstituted C6 to C30 aryl group, a substituted or unsubstituted dibenzofuranyl group, or a substituted or unsubstituted dibenzothiophenyl group, and
A is a ring of Group I, 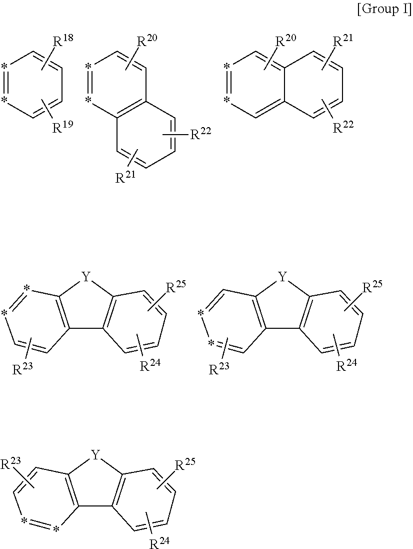
wherein, in Group I,
each * is a linking carbon,
Y is O or S, and
R¹⁸ to R²⁵ are each independently hydrogen, deuterium, a substituted or unsubstituted C1 to C30 alkyl group, a substituted or unsubstituted C6 to C30 aryl group, or a substituted or unsubstituted C2 to C30 heterocyclic group.

2. The composition as claimed in claim 1, wherein ET of Chemical Formula 1 is a substituted or unsubstituted pyrimidinyl group, a substituted or unsubstituted triazinyl group, a substituted or unsubstituted quinazolinyl group, a substituted or unsubstituted quinoxalinyl group, a substituted or unsubstituted benzofuranpyrimidinyl group, or a substituted or unsubstituted benzothiophenepyrimidinyl group.

3. The composition as claimed in claim 1, wherein ET of Chemical Formula 1 is a group of Group II: 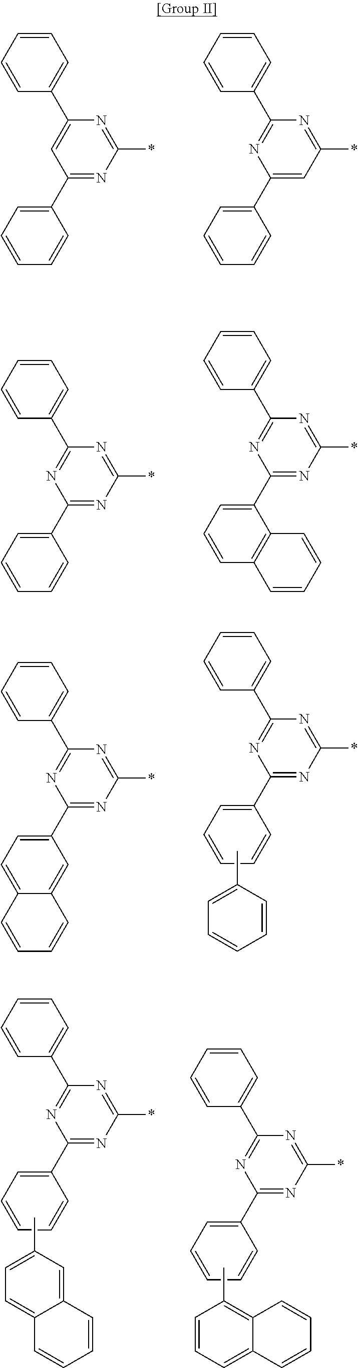 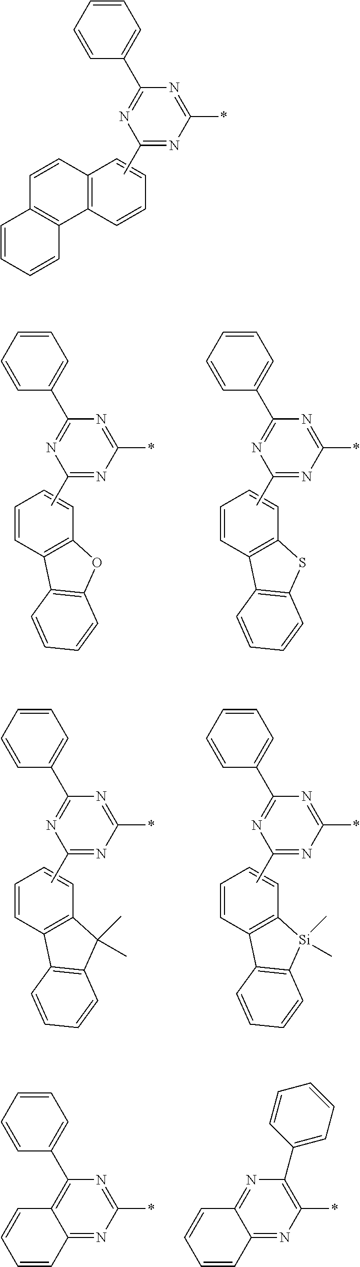 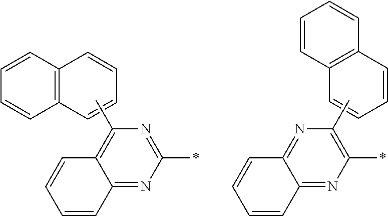
wherein, in Group II, * is a linking point.

4. The composition as claimed in claim 1, wherein the first compound is a compound of Group 1:

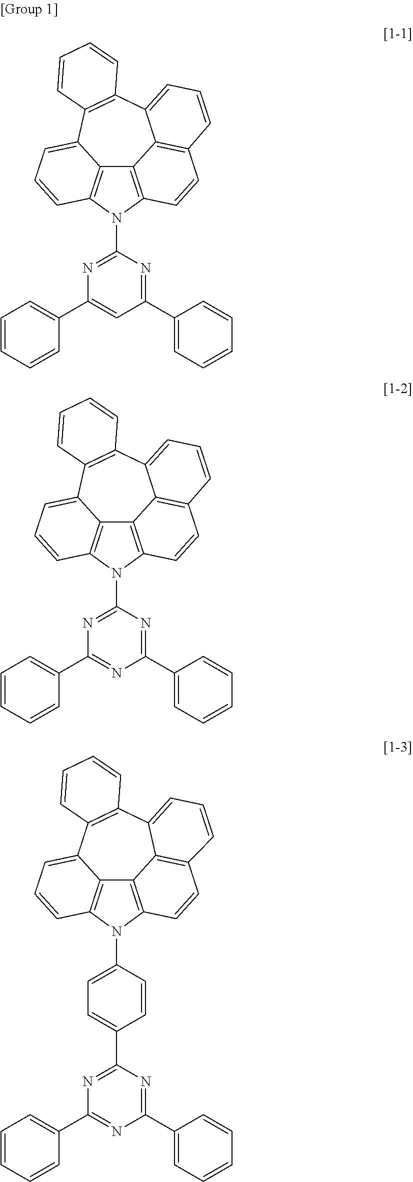 [SMILES: c1ccc(-c2cc(-c3ccccc3)nc(-n3c4cccc5c4c4c6c(cccc6ccc43)-c3ccccc3-5)n2)cc1 ; c1ccc(-c2nc(-c3ccccc3)nc(-n3c4cccc5c4c4c6c(cccc6ccc43)-c3ccccc3-5)n2)cc1 ; c1ccc(-c2nc(-c3ccccc3)nc(-c3ccc(-n4c5cccc6c5c5c7c(cccc7ccc54)-c4ccccc4-6)cc3)n2)cc1]

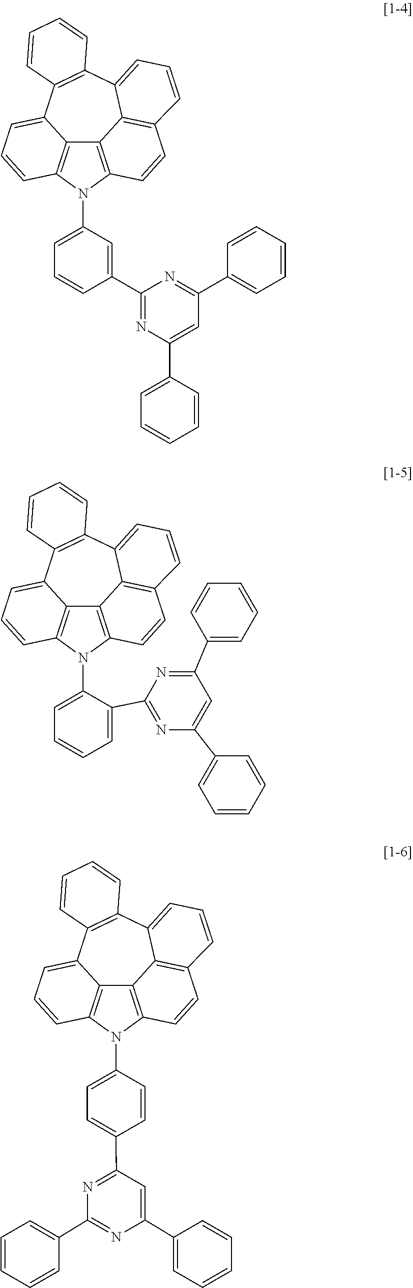 [SMILES: c1ccc(-c2cc(-c3ccccc3)nc(-c3cccc(-n4c5cccc6c5c5c7c(cccc7ccc54)-c4ccccc4-6)c3)n2)cc1 ; c1ccc(-c2cc(-c3ccccc3)nc(-c3ccccc3-n3c4cccc5c4c4c6c(cccc6ccc43)-c3ccccc3-5)n2)cc1 ; c1ccc(-c2cc(-c3ccc(-n4c5cccc6c5c5c7c(cccc7ccc54)-c4ccccc4-6)cc3)nc(-c3ccccc3)n2)cc1]

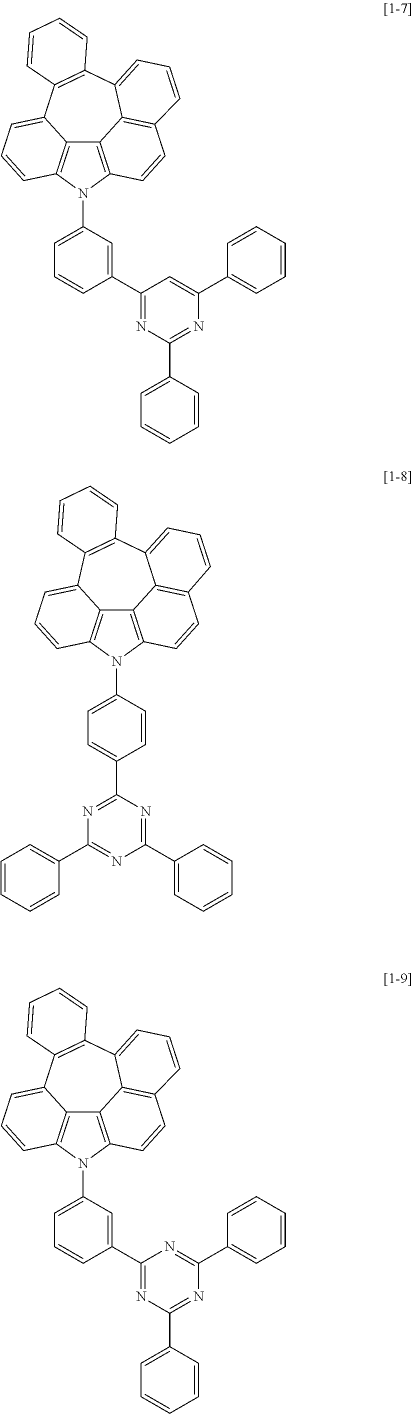 [SMILES: c1ccc(-c2cc(-c3cccc(-n4c5cccc6c5c5c7c(cccc7ccc54)-c4ccccc4-6)c3)nc(-c3ccccc3)n2)cc1 ; c1ccc(-c2nc(-c3ccccc3)nc(-c3ccc(-n4c5cccc6c5c5c7c(cccc7ccc54)-c4ccccc4-6)cc3)n2)cc1 ; c1ccc(-c2nc(-c3ccccc3)nc(-c3cccc(-n4c5cccc6c5c5c7c(cccc7ccc54)-c4ccccc4-6)c3)n2)cc1]

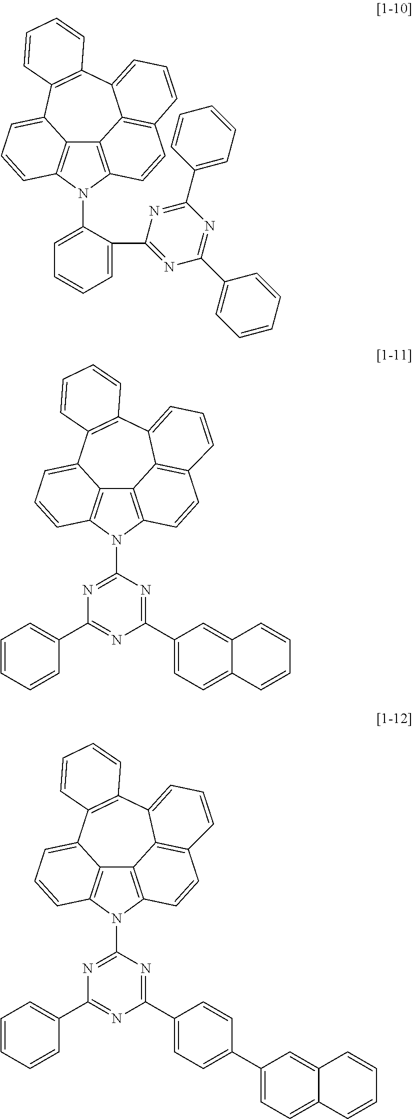 [SMILES: c1ccc(-c2nc(-c3ccccc3)nc(-c3ccccc3-n3c4cccc5c4c4c6c(cccc6ccc43)-c3ccccc3-5)n2)cc1 ; c1ccc(-c2nc(-c3ccc4ccccc4c3)nc(-n3c4cccc5c4c4c6c(cccc6ccc43)-c3ccccc3-5)n2)cc1 ; c1ccc(-c2nc(-c3ccc(-c4ccc5ccccc5c4)cc3)nc(-n3c4cccc5c4c4c6c(cccc6ccc43)-c3ccccc3-5)n2)cc1]

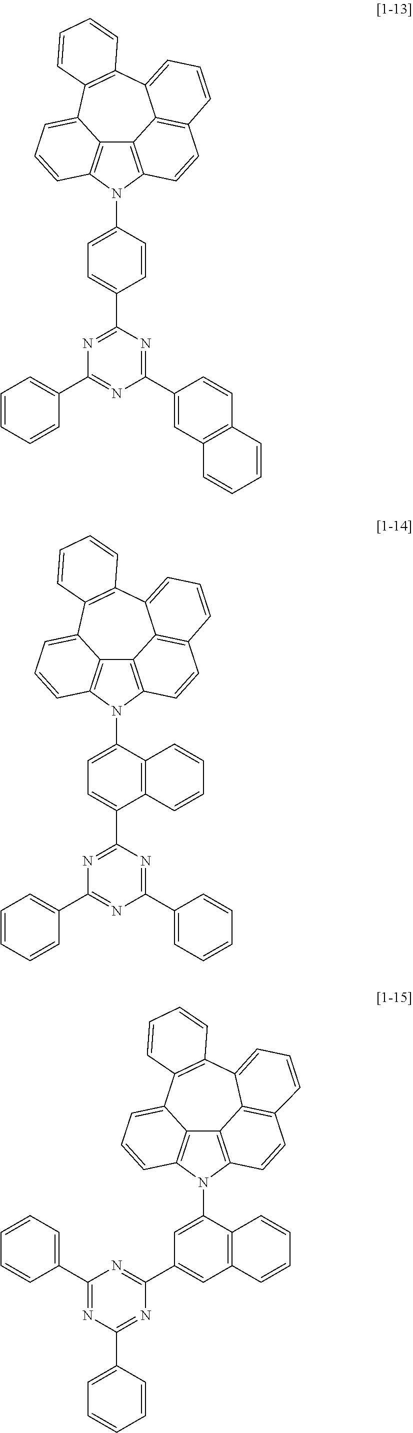 [SMILES: c1ccc(-c2nc(-c3ccc(-n4c5cccc6c5c5c7c(cccc7ccc54)-c4ccccc4-6)cc3)nc(-c3ccc4ccccc4c3)n2)cc1 ; c1ccc(-c2nc(-c3ccccc3)nc(-c3ccc(-n4c5cccc6c5c5c7c(cccc7ccc54)-c4ccccc4-6)c4ccccc34)n2)cc1 ; c1ccc(-c2nc(-c3ccccc3)nc(-c3cc(-n4c5cccc6c5c5c7c(cccc7ccc54)-c4ccccc4-6)c4ccccc4c3)n2)cc1]

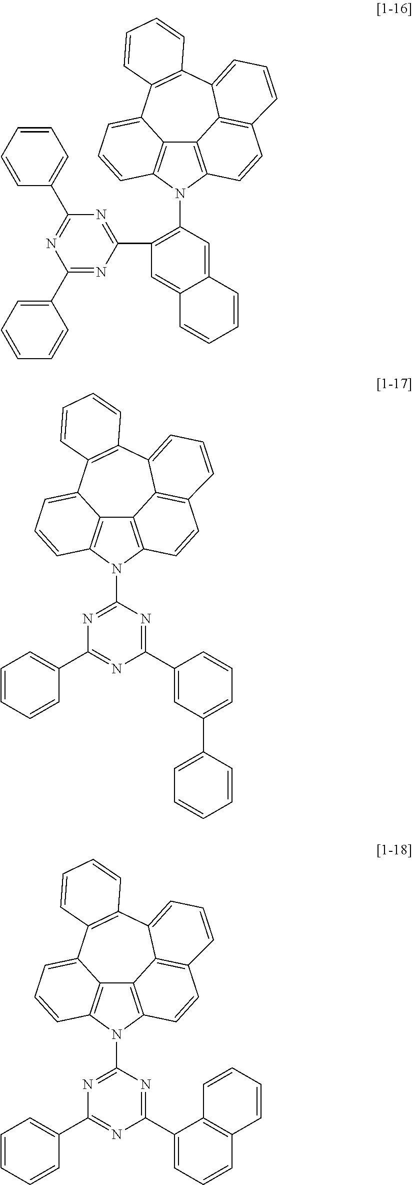 [SMILES: c1ccc(-c2nc(-c3ccccc3)nc(-c3cc4ccccc4cc3-n3c4cccc5c4c4c6c(cccc6ccc43)-c3ccccc3-5)n2)cc1 ; c1ccc(-c2cccc(-c3nc(-c4ccccc4)nc(-n4c5cccc6c5c5c7c(cccc7ccc54)-c4ccccc4-6)n3)c2)cc1 ; c1ccc(-c2nc(-c3cccc4ccccc34)nc(-n3c4cccc5c4c4c6c(cccc6ccc43)-c3ccccc3-5)n2)cc1]

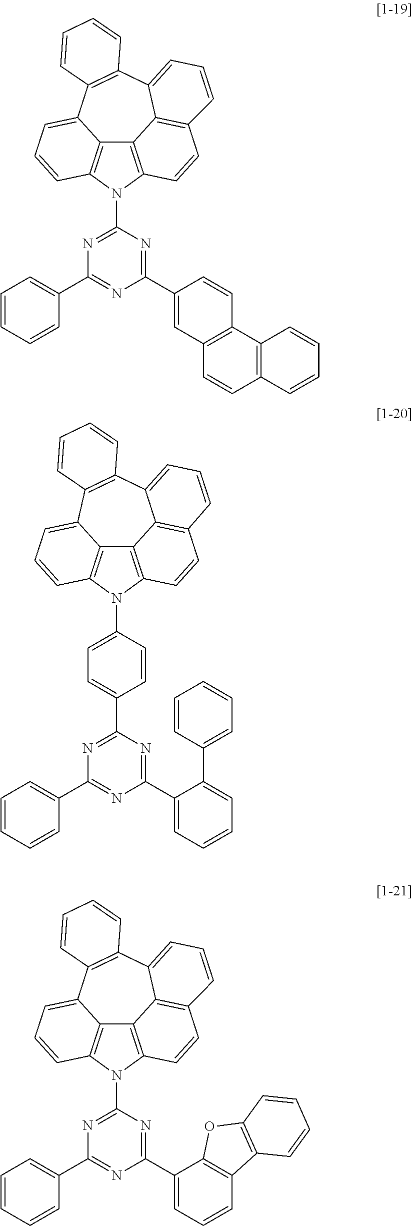 [SMILES: c1ccc(-c2nc(-c3ccc4c(ccc5ccccc54)c3)nc(-n3c4cccc5c4c4c6c(cccc6ccc43)-c3ccccc3-5)n2)cc1 ; c1ccc(-c2nc(-c3ccc(-n4c5cccc6c5c5c7c(cccc7ccc54)-c4ccccc4-6)cc3)nc(-c3ccccc3-c3ccccc3)n2)cc1 ; c1ccc(-c2nc(-c3cccc4c3oc3ccccc34)nc(-n3c4cccc5c4c4c6c(cccc6ccc43)-c3ccccc3-5)n2)cc1]

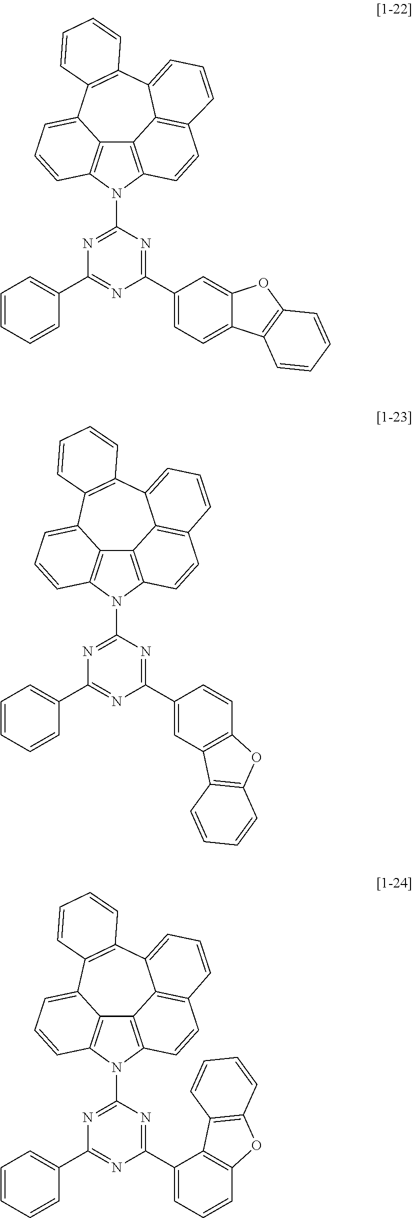 [SMILES: c1ccc(-c2nc(-c3ccc4c(c3)oc3ccccc34)nc(-n3c4cccc5c4c4c6c(cccc6ccc43)-c3ccccc3-5)n2)cc1 ; c1ccc(-c2nc(-c3ccc4oc5ccccc5c4c3)nc(-n3c4cccc5c4c4c6c(cccc6ccc43)-c3ccccc3-5)n2)cc1 ; c1ccc(-c2nc(-c3cccc4oc5ccccc5c34)nc(-n3c4cccc5c4c4c6c(cccc6ccc43)-c3ccccc3-5)n2)cc1]

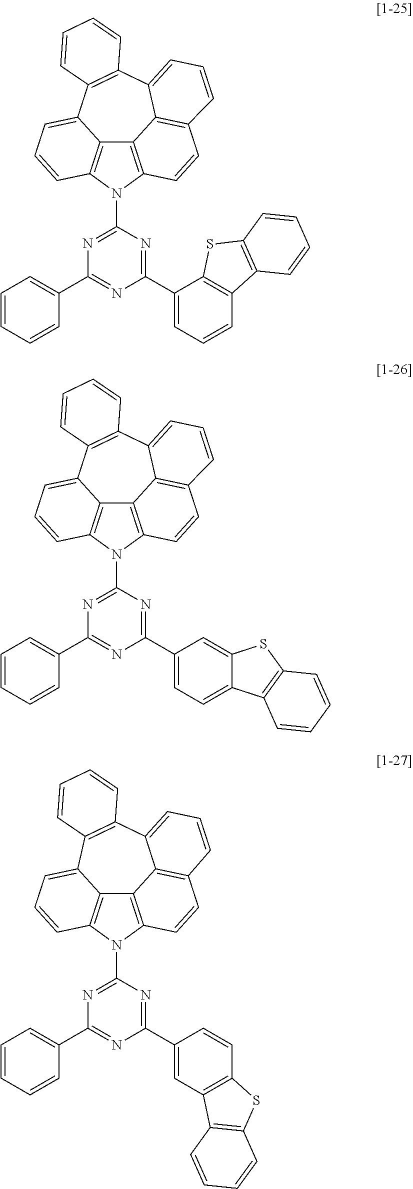 [SMILES: c1ccc(-c2nc(-c3cccc4c3sc3ccccc34)nc(-n3c4cccc5c4c4c6c(cccc6ccc43)-c3ccccc3-5)n2)cc1 ; c1ccc(-c2nc(-c3ccc4c(c3)sc3ccccc34)nc(-n3c4cccc5c4c4c6c(cccc6ccc43)-c3ccccc3-5)n2)cc1 ; c1ccc(-c2nc(-c3ccc4sc5ccccc5c4c3)nc(-n3c4cccc5c4c4c6c(cccc6ccc43)-c3ccccc3-5)n2)cc1]

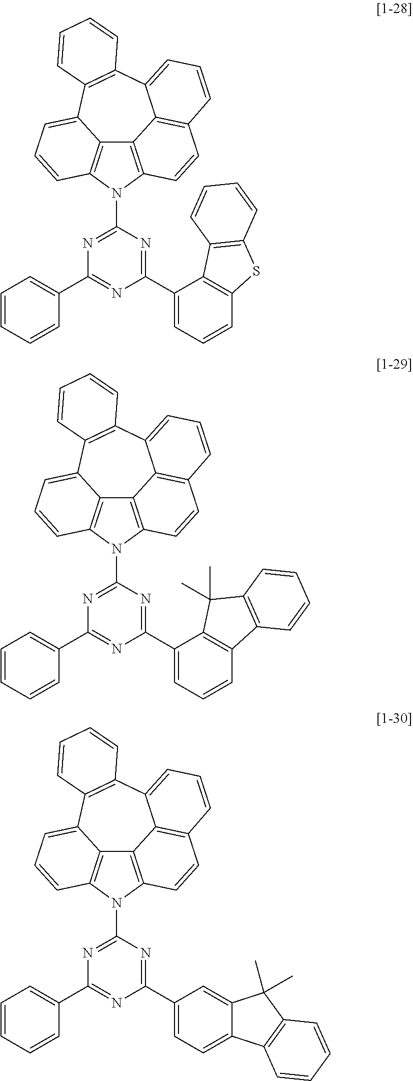 [SMILES: c1ccc(-c2nc(-c3cccc4sc5ccccc5c34)nc(-n3c4cccc5c4c4c6c(cccc6ccc43)-c3ccccc3-5)n2)cc1 ; CC1(C)c2ccccc2-c2cccc(-c3nc(-c4ccccc4)nc(-n4c5cccc6c5c5c7c(cccc7ccc54)-c4ccccc4-6)n3)c21 ; CC1(C)c2ccccc2-c2ccc(-c3nc(-c4ccccc4)nc(-n4c5cccc6c5c5c7c(cccc7ccc54)-c4ccccc4-6)n3)cc21]

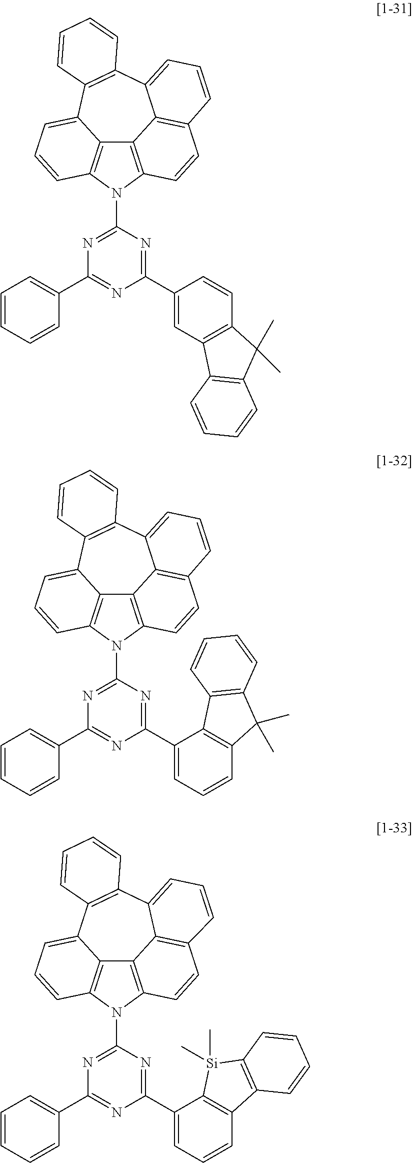 [SMILES: CC1(C)c2ccccc2-c2cc(-c3nc(-c4ccccc4)nc(-n4c5cccc6c5c5c7c(cccc7ccc54)-c4ccccc4-6)n3)ccc21 ; CC1(C)c2ccccc2-c2c(-c3nc(-c4ccccc4)nc(-n4c5cccc6c5c5c7c(cccc7ccc54)-c4ccccc4-6)n3)cccc21 ; C[Si]1(C)c2ccccc2-c2cccc(-c3nc(-c4ccccc4)nc(-n4c5cccc6c5c5c7c(cccc7ccc54)-c4ccccc4-6)n3)c21]

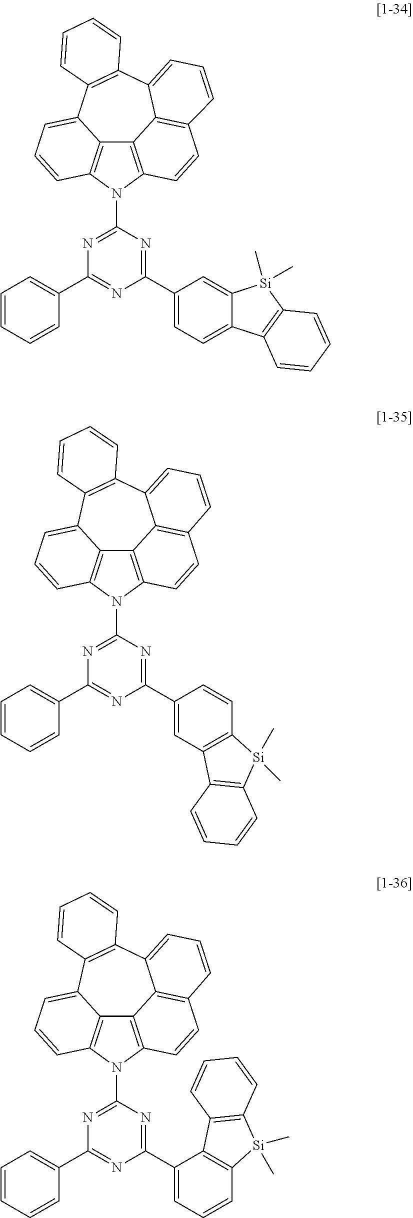 [SMILES: C[Si]1(C)c2ccccc2-c2ccc(-c3nc(-c4ccccc4)nc(-n4c5cccc6c5c5c7c(cccc7ccc54)-c4ccccc4-6)n3)cc21 ; C[Si]1(C)c2ccccc2-c2cc(-c3nc(-c4ccccc4)nc(-n4c5cccc6c5c5c7c(cccc7ccc54)-c4ccccc4-6)n3)ccc21 ; C[Si]1(C)c2ccccc2-c2c(-c3nc(-c4ccccc4)nc(-n4c5cccc6c5c5c7c(cccc7ccc54)-c4ccccc4-6)n3)cccc21]

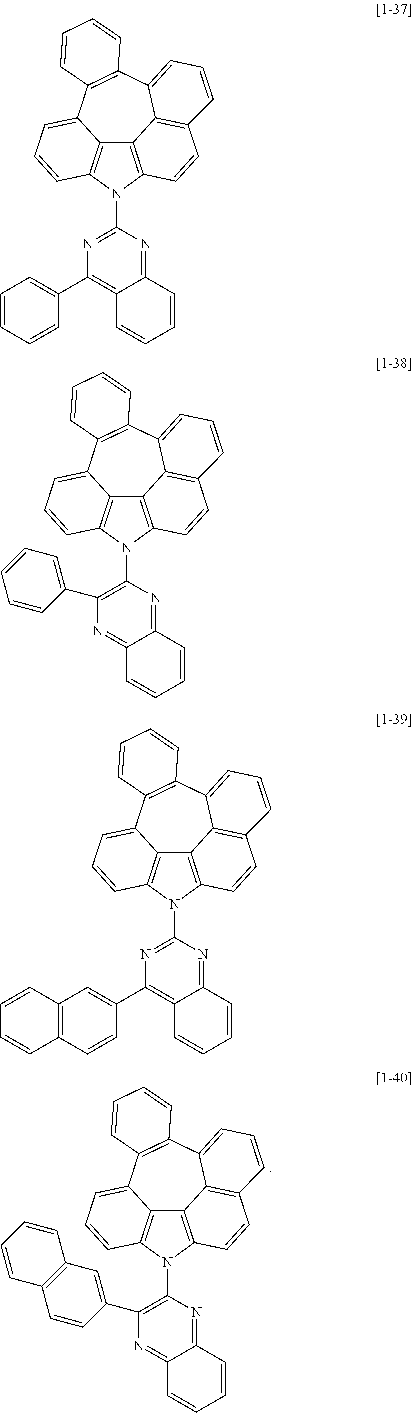

5. The composition as claimed in claim 1, wherein:
the second compound is represented by one of Chemical Formula 2A to Chemical Formula 2J, 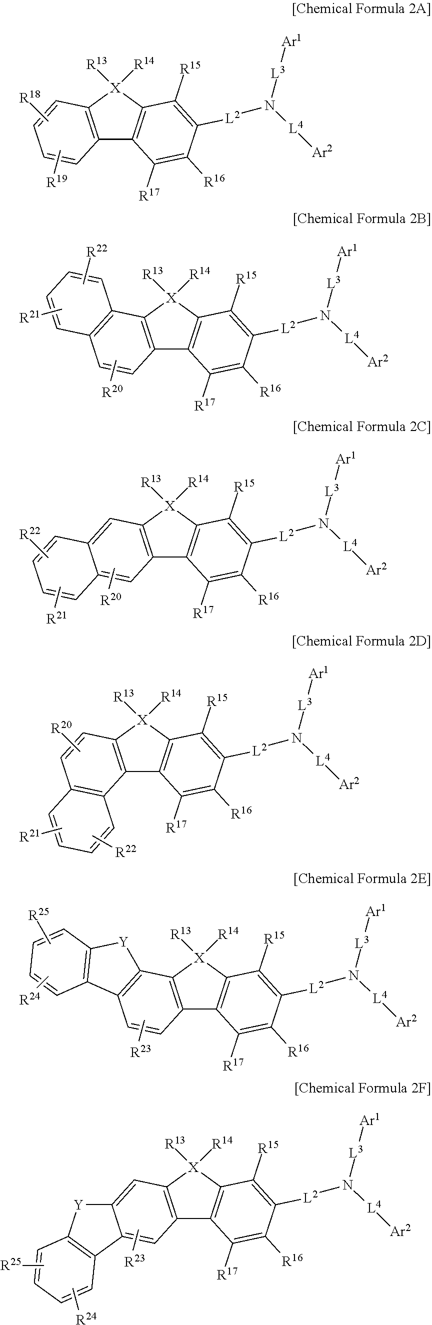 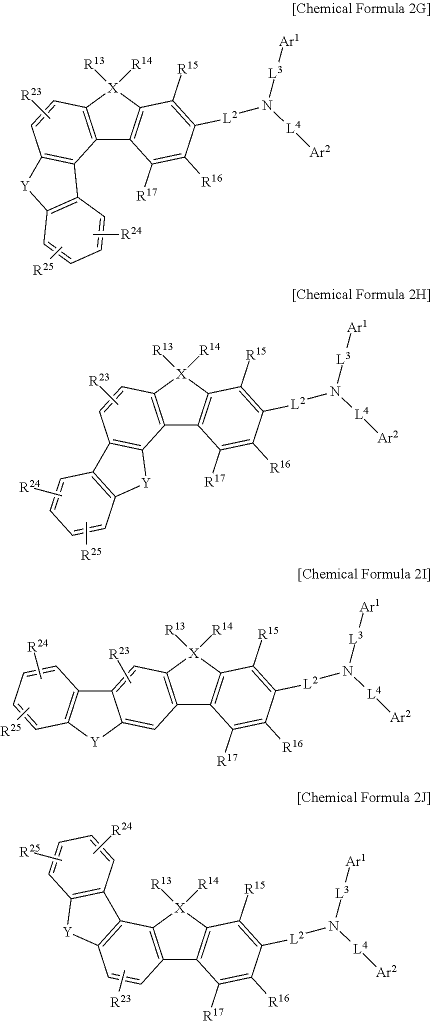
in Chemical Formula 2A to Chemical Formula 2J, X, Y, R¹³ to R²⁵, L² to L⁴, Ar¹, and Ar² are defined the same as those of Chemical Formula 2.

6. The composition as claimed in claim 1, wherein:
L² is a single bond or a substituted or unsubstituted phenylene group, and
L³ and L⁴ are each independently a single bond, a substituted or unsubstituted phenylene group, a substituted or unsubstituted biphenylene group, a substituted or unsubstituted naphthylene group, or a substituted or unsubstituted fluorenylene group.

7. The composition as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted anthracenyl group, a substituted or unsubstituted phenanthrenyl group, a substituted or unsubstituted triphenylene group, a substituted or unsubstituted dibenzofuranyl group, or a substituted or unsubstituted dibenzothiophenyl group.

8. The composition as claimed in claim 1, wherein moieties *-L³-Ar¹ and *-L⁴-Ar² of Chemical Formula 2 are each independently a moiety of Group III: 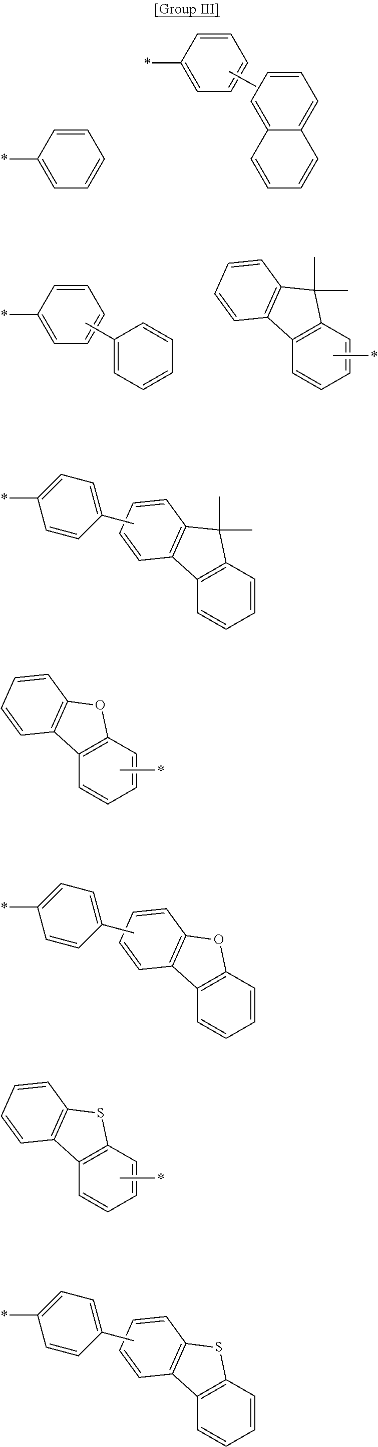 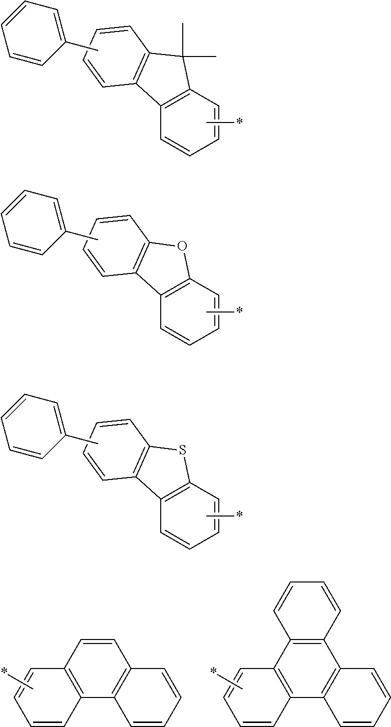
wherein, in Group III, * is a linking point with N of Chemical Formula 2.

9. The composition as claimed in claim 1, wherein the second compound is a compound of Group 2,

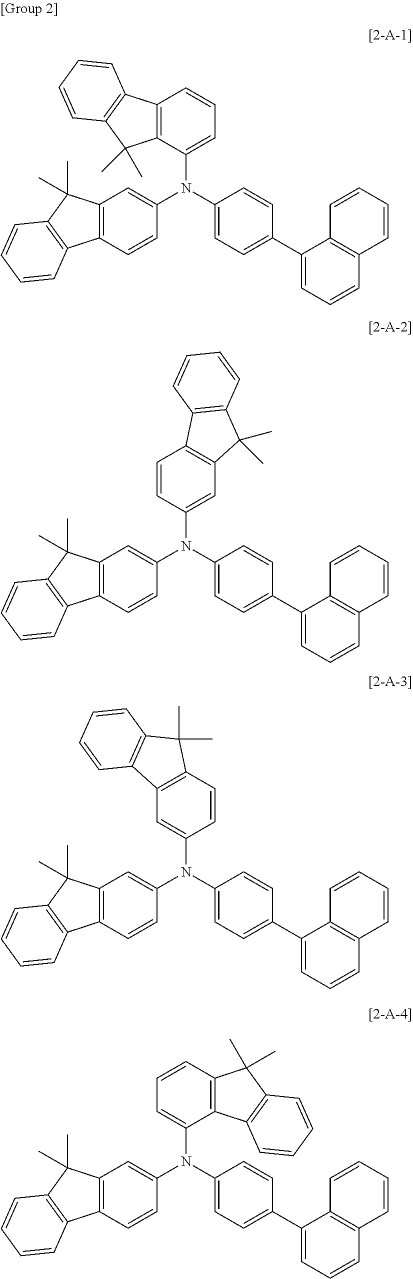 [SMILES: CC1(C)c2ccccc2-c2ccc(N(c3ccc(-c4cccc5ccccc45)cc3)c3cccc4c3C(C)(C)c3ccccc3-4)cc21 ; CC1(C)c2ccccc2-c2ccc(N(c3ccc(-c4cccc5ccccc45)cc3)c3ccc4c(c3)C(C)(C)c3ccccc3-4)cc21 ; CC1(C)c2ccccc2-c2cc(N(c3ccc(-c4cccc5ccccc45)cc3)c3ccc4c(c3)C(C)(C)c3ccccc3-4)ccc21 ; CC1(C)c2ccccc2-c2ccc(N(c3ccc(-c4cccc5ccccc45)cc3)c3cccc4c3-c3ccccc3C4(C)C)cc21]

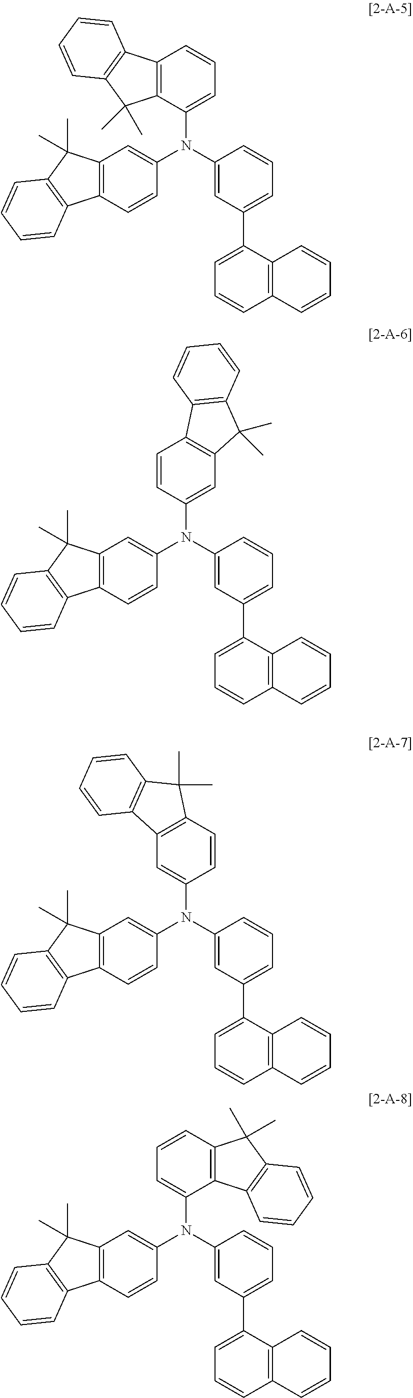

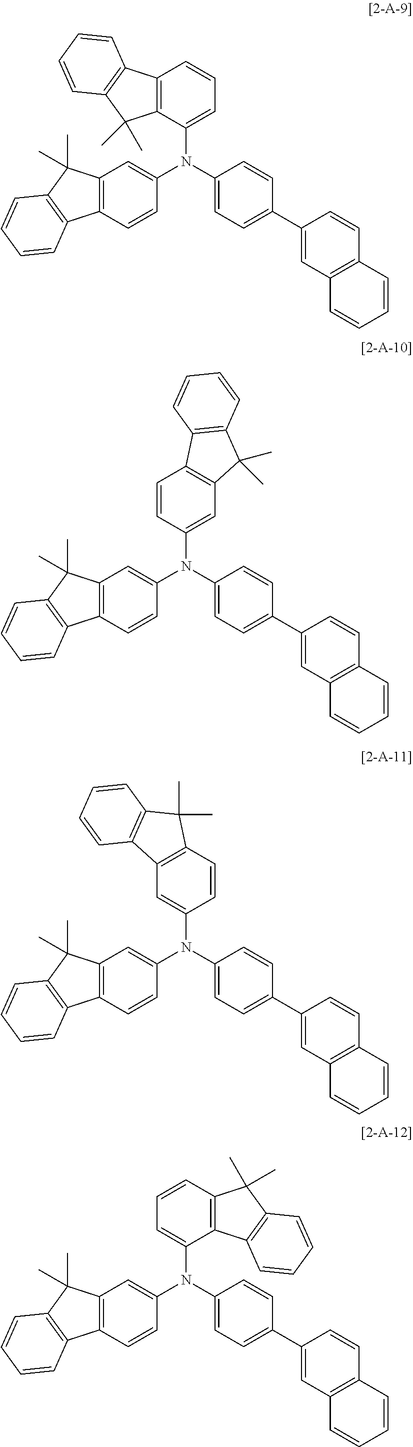 [SMILES: CC1(C)c2ccccc2-c2ccc(N(c3ccc(-c4ccc5ccccc5c4)cc3)c3cccc4c3C(C)(C)c3ccccc3-4)cc21 ; CC1(C)c2ccccc2-c2ccc(N(c3ccc(-c4ccc5ccccc5c4)cc3)c3ccc4c(c3)C(C)(C)c3ccccc3-4)cc21 ; CC1(C)c2ccccc2-c2cc(N(c3ccc(-c4ccc5ccccc5c4)cc3)c3ccc4c(c3)C(C)(C)c3ccccc3-4)ccc21 ; CC1(C)c2ccccc2-c2ccc(N(c3ccc(-c4ccc5ccccc5c4)cc3)c3cccc4c3-c3ccccc3C4(C)C)cc21]

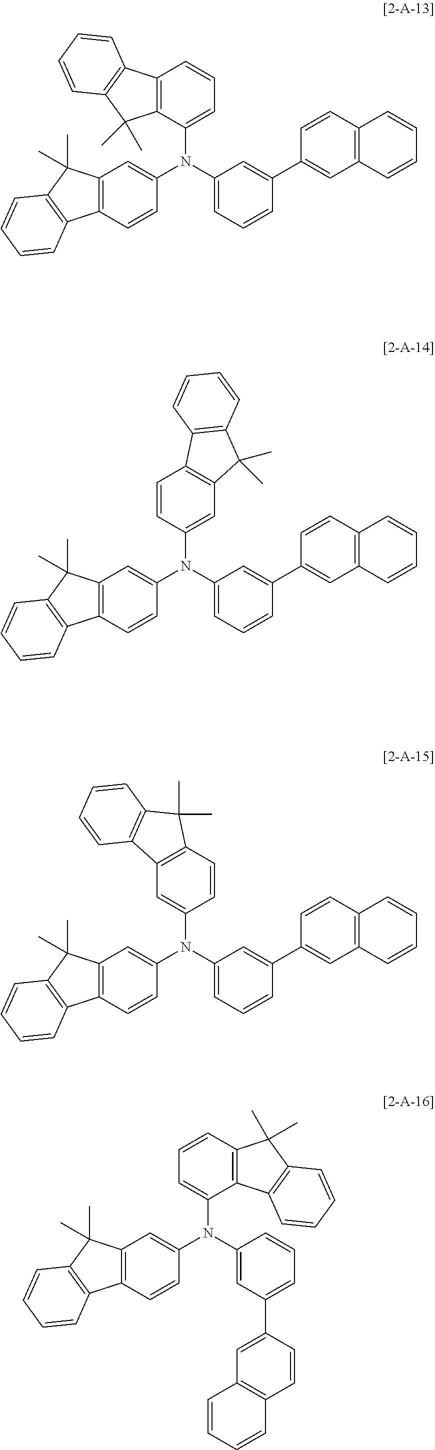 [SMILES: CC1(C)c2ccccc2-c2ccc(N(c3cccc(-c4ccc5ccccc5c4)c3)c3cccc4c3C(C)(C)c3ccccc3-4)cc21 ; CC1(C)c2ccccc2-c2ccc(N(c3cccc(-c4ccc5ccccc5c4)c3)c3ccc4c(c3)C(C)(C)c3ccccc3-4)cc21 ; CC1(C)c2ccccc2-c2cc(N(c3cccc(-c4ccc5ccccc5c4)c3)c3ccc4c(c3)C(C)(C)c3ccccc3-4)ccc21 ; CC1(C)c2ccccc2-c2ccc(N(c3cccc(-c4ccc5ccccc5c4)c3)c3cccc4c3-c3ccccc3C4(C)C)cc21]

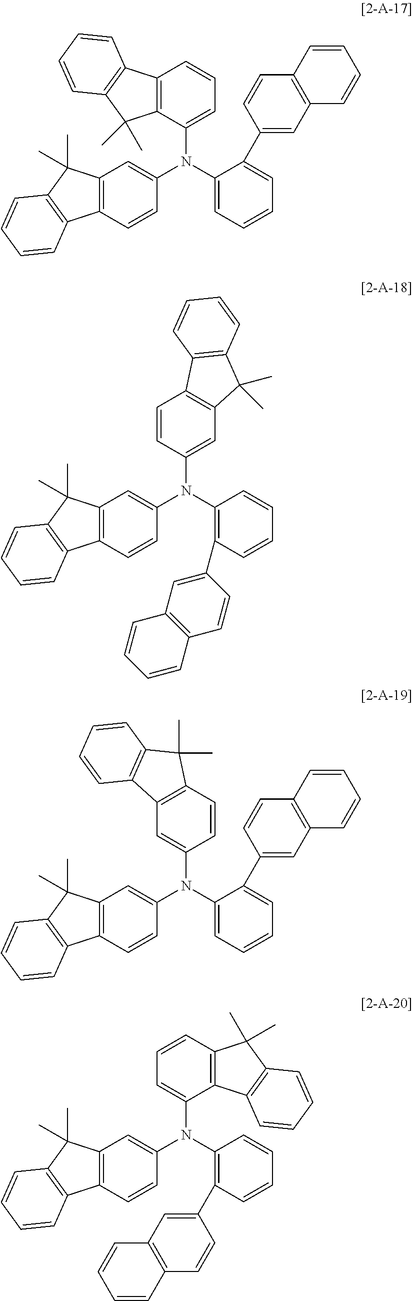 [SMILES: CC1(C)c2ccccc2-c2ccc(N(c3ccccc3-c3ccc4ccccc4c3)c3cccc4c3C(C)(C)c3ccccc3-4)cc21 ; CC1(C)c2ccccc2-c2ccc(N(c3ccc4c(c3)C(C)(C)c3ccccc3-4)c3ccccc3-c3ccc4ccccc4c3)cc21 ; CC1(C)c2ccccc2-c2cc(N(c3ccc4c(c3)C(C)(C)c3ccccc3-4)c3ccccc3-c3ccc4ccccc4c3)ccc21 ; CC1(C)c2ccccc2-c2ccc(N(c3ccccc3-c3ccc4ccccc4c3)c3cccc4c3-c3ccccc3C4(C)C)cc21]

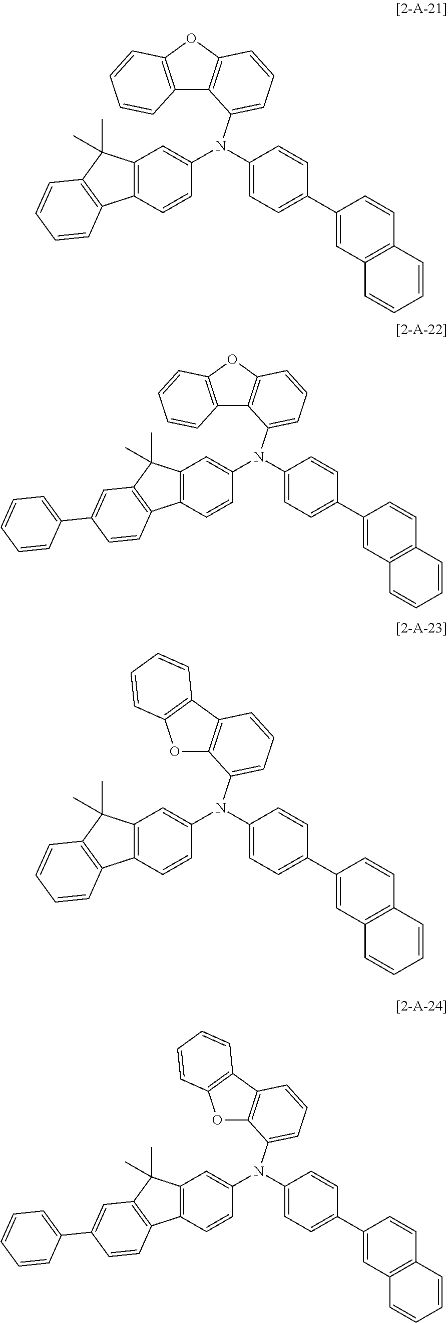 [SMILES: CC1(C)c2ccccc2-c2ccc(N(c3ccc(-c4ccc5ccccc5c4)cc3)c3cccc4oc5ccccc5c34)cc21 ; CC1(C)c2cc(-c3ccccc3)ccc2-c2ccc(N(c3ccc(-c4ccc5ccccc5c4)cc3)c3cccc4oc5ccccc5c34)cc21 ; CC1(C)c2ccccc2-c2ccc(N(c3ccc(-c4ccc5ccccc5c4)cc3)c3cccc4c3oc3ccccc34)cc21 ; CC1(C)c2cc(-c3ccccc3)ccc2-c2ccc(N(c3ccc(-c4ccc5ccccc5c4)cc3)c3cccc4c3oc3ccccc34)cc21]

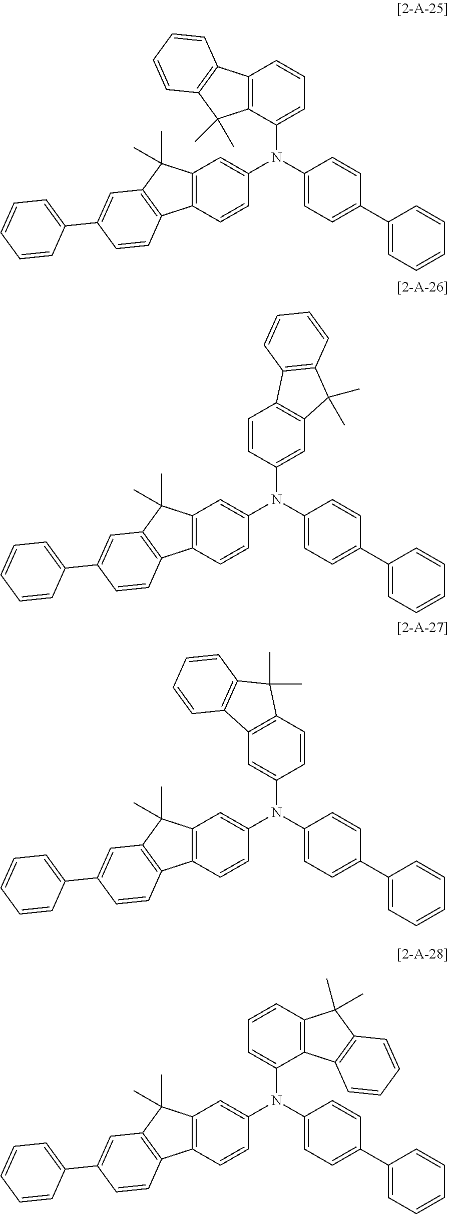 [SMILES: CC1(C)c2cc(-c3ccccc3)ccc2-c2ccc(N(c3ccc(-c4ccccc4)cc3)c3cccc4c3C(C)(C)c3ccccc3-4)cc21 ; CC1(C)c2ccccc2-c2ccc(N(c3ccc(-c4ccccc4)cc3)c3ccc4c(c3)C(C)(C)c3cc(-c5ccccc5)ccc3-4)cc21 ; CC1(C)c2ccccc2-c2cc(N(c3ccc(-c4ccccc4)cc3)c3ccc4c(c3)C(C)(C)c3cc(-c5ccccc5)ccc3-4)ccc21 ; CC1(C)c2cc(-c3ccccc3)ccc2-c2ccc(N(c3ccc(-c4ccccc4)cc3)c3cccc4c3-c3ccccc3C4(C)C)cc21]

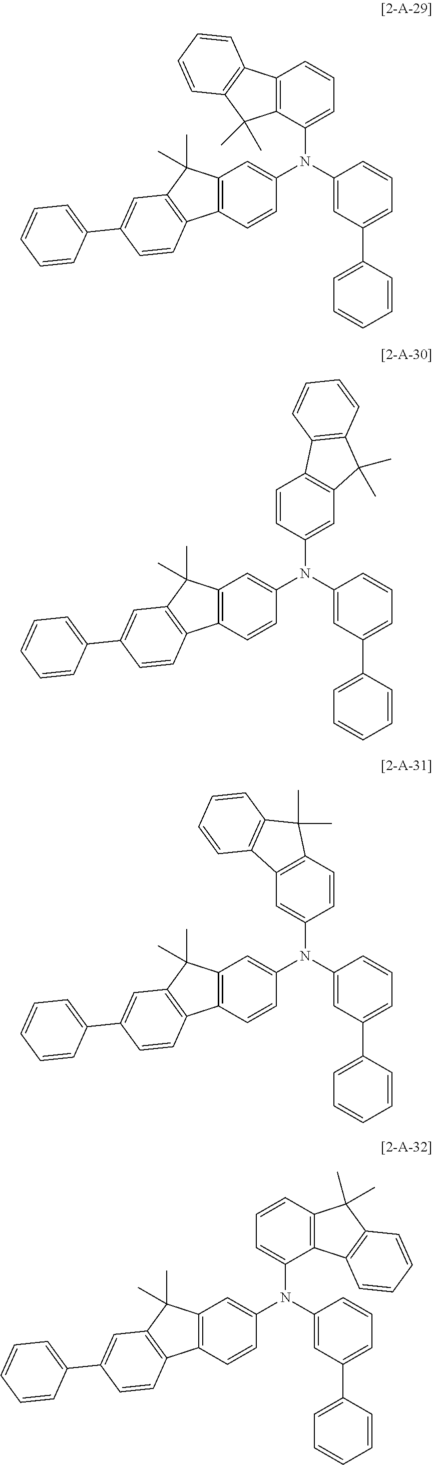 [SMILES: CC1(C)c2cc(-c3ccccc3)ccc2-c2ccc(N(c3cccc(-c4ccccc4)c3)c3cccc4c3C(C)(C)c3ccccc3-4)cc21 ; CC1(C)c2ccccc2-c2ccc(N(c3cccc(-c4ccccc4)c3)c3ccc4c(c3)C(C)(C)c3cc(-c5ccccc5)ccc3-4)cc21 ; CC1(C)c2ccccc2-c2cc(N(c3cccc(-c4ccccc4)c3)c3ccc4c(c3)C(C)(C)c3cc(-c5ccccc5)ccc3-4)ccc21 ; CC1(C)c2cc(-c3ccccc3)ccc2-c2ccc(N(c3cccc(-c4ccccc4)c3)c3cccc4c3-c3ccccc3C4(C)C)cc21]

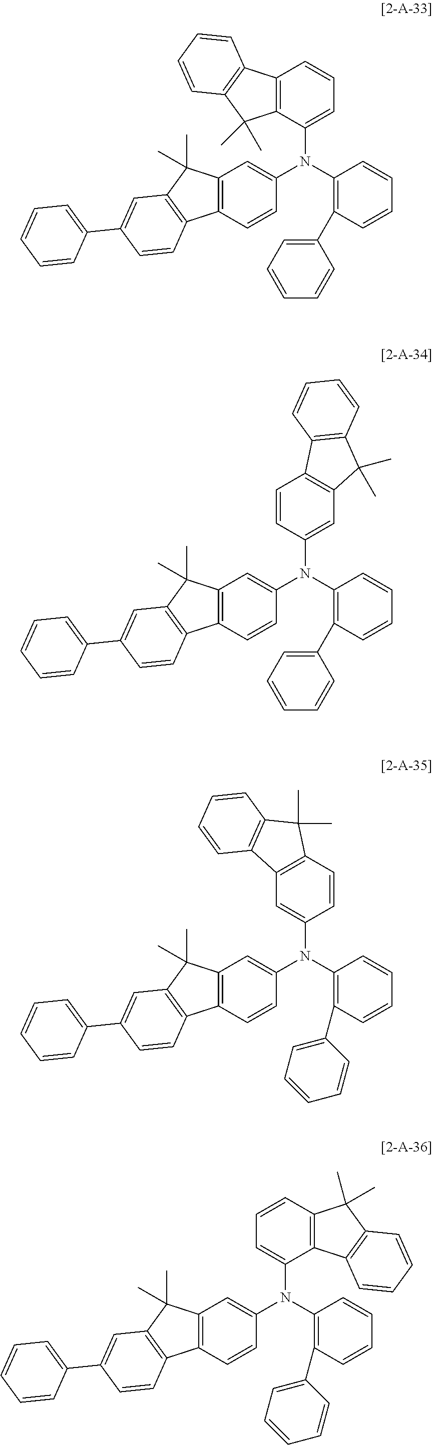 [SMILES: CC1(C)c2cc(-c3ccccc3)ccc2-c2ccc(N(c3ccccc3-c3ccccc3)c3cccc4c3C(C)(C)c3ccccc3-4)cc21 ; CC1(C)c2ccccc2-c2ccc(N(c3ccc4c(c3)C(C)(C)c3cc(-c5ccccc5)ccc3-4)c3ccccc3-c3ccccc3)cc21 ; CC1(C)c2ccccc2-c2cc(N(c3ccc4c(c3)C(C)(C)c3cc(-c5ccccc5)ccc3-4)c3ccccc3-c3ccccc3)ccc21 ; CC1(C)c2cc(-c3ccccc3)ccc2-c2ccc(N(c3ccccc3-c3ccccc3)c3cccc4c3-c3ccccc3C4(C)C)cc21]

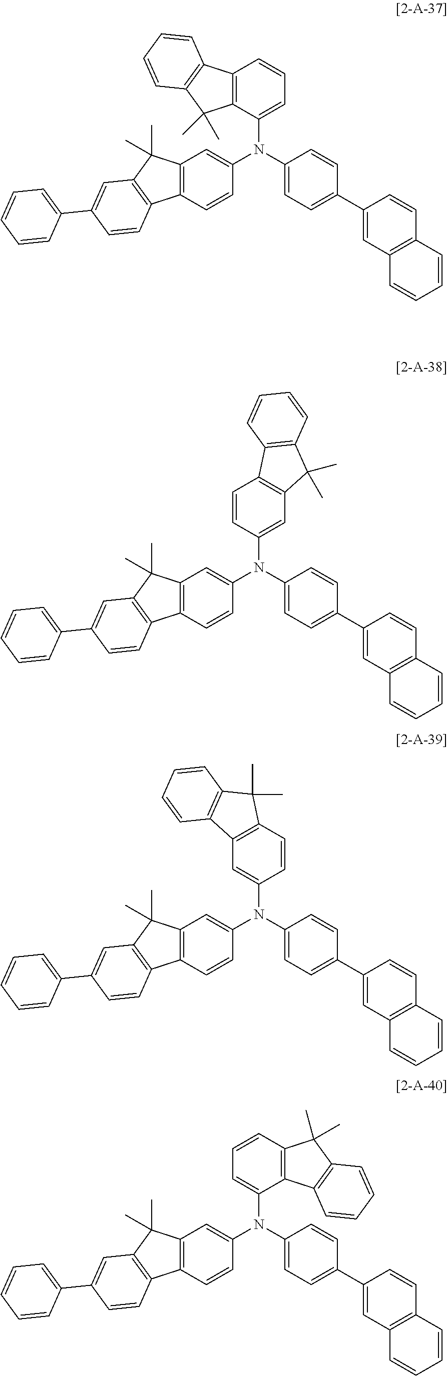 [SMILES: CC1(C)c2cc(-c3ccccc3)ccc2-c2ccc(N(c3ccc(-c4ccc5ccccc5c4)cc3)c3cccc4c3C(C)(C)c3ccccc3-4)cc21 ; CC1(C)c2ccccc2-c2ccc(N(c3ccc(-c4ccc5ccccc5c4)cc3)c3ccc4c(c3)C(C)(C)c3cc(-c5ccccc5)ccc3-4)cc21 ; CC1(C)c2ccccc2-c2cc(N(c3ccc(-c4ccc5ccccc5c4)cc3)c3ccc4c(c3)C(C)(C)c3cc(-c5ccccc5)ccc3-4)ccc21 ; CC1(C)c2cc(-c3ccccc3)ccc2-c2ccc(N(c3ccc(-c4ccc5ccccc5c4)cc3)c3cccc4c3-c3ccccc3C4(C)C)cc21]

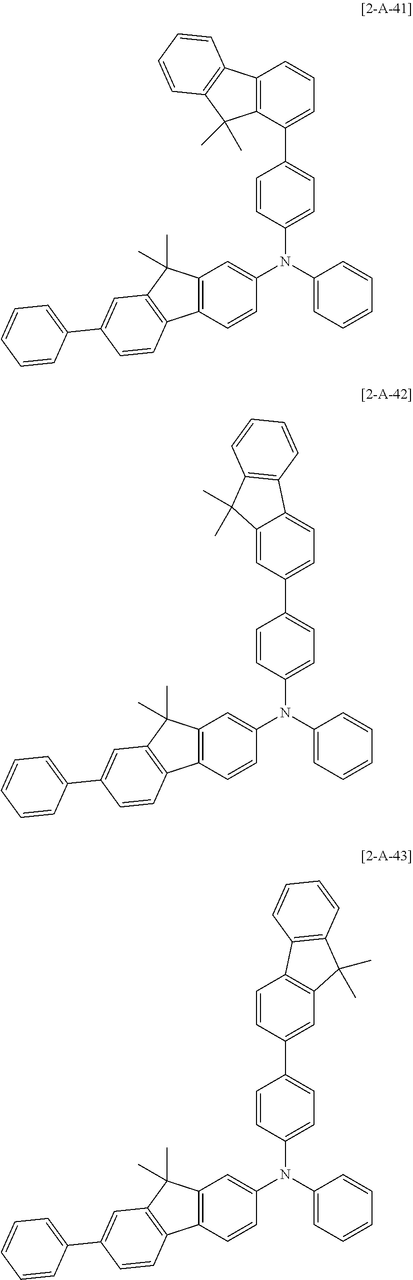 [SMILES: CC1(C)c2cc(-c3ccccc3)ccc2-c2ccc(N(c3ccccc3)c3ccc(-c4cccc5c4C(C)(C)c4ccccc4-5)cc3)cc21 ; CC1(C)c2ccccc2-c2ccc(-c3ccc(N(c4ccccc4)c4ccc5c(c4)C(C)(C)c4cc(-c6ccccc6)ccc4-5)cc3)cc21 ; CC1(C)c2ccccc2-c2ccc(-c3ccc(N(c4ccccc4)c4ccc5c(c4)C(C)(C)c4cc(-c6ccccc6)ccc4-5)cc3)cc21]

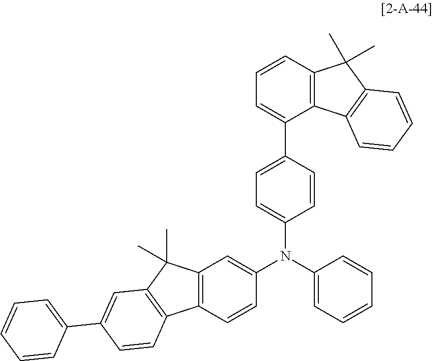 [SMILES: CC1(C)c2cc(-c3ccccc3)ccc2-c2ccc(N(c3ccccc3)c3ccc(-c4cccc5c4-c4ccccc4C5(C)C)cc3)cc21]

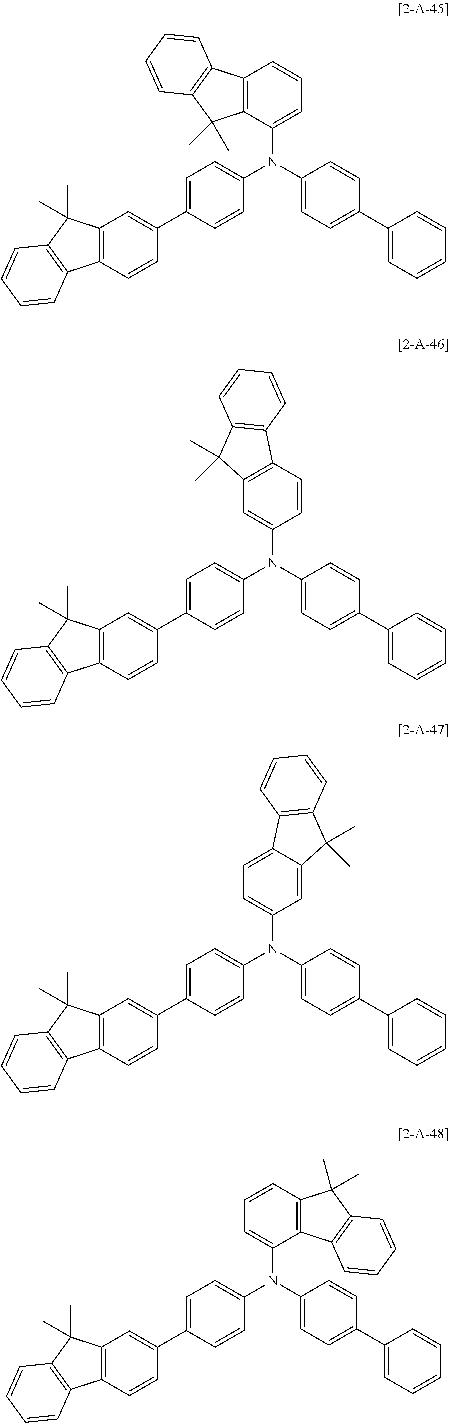 [SMILES: CC1(C)c2ccccc2-c2ccc(-c3ccc(N(c4ccc(-c5ccccc5)cc4)c4cccc5c4C(C)(C)c4ccccc4-5)cc3)cc21 ; CC1(C)c2ccccc2-c2ccc(-c3ccc(N(c4ccc(-c5ccccc5)cc4)c4ccc5c(c4)C(C)(C)c4ccccc4-5)cc3)cc21 ; CC1(C)c2ccccc2-c2ccc(-c3ccc(N(c4ccc(-c5ccccc5)cc4)c4ccc5c(c4)C(C)(C)c4ccccc4-5)cc3)cc21 ; CC1(C)c2ccccc2-c2ccc(-c3ccc(N(c4ccc(-c5ccccc5)cc4)c4cccc5c4-c4ccccc4C5(C)C)cc3)cc21]

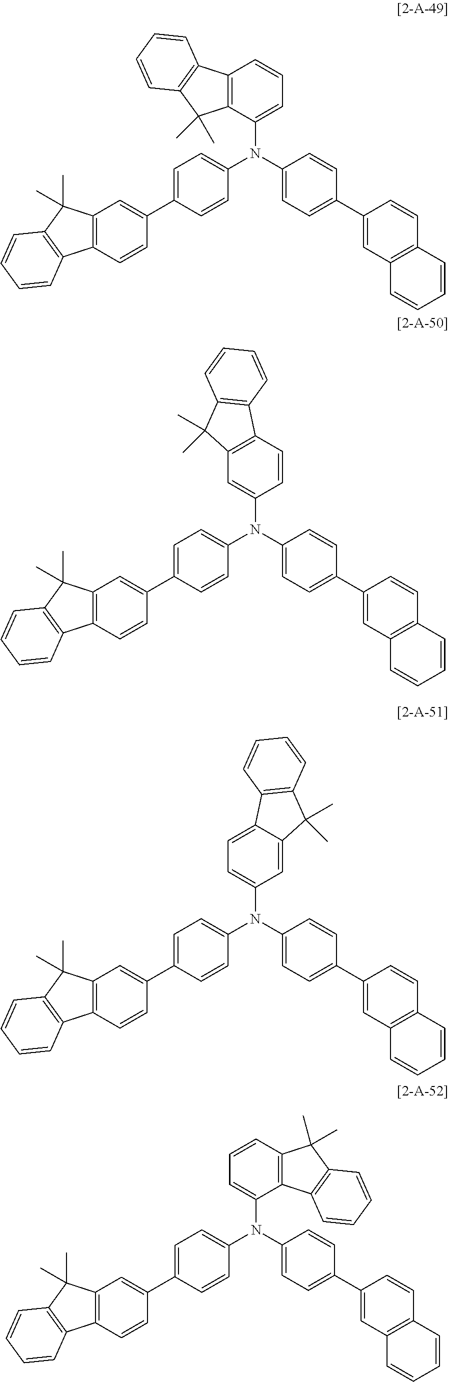 [SMILES: CC1(C)c2ccccc2-c2ccc(-c3ccc(N(c4ccc(-c5ccc6ccccc6c5)cc4)c4cccc5c4C(C)(C)c4ccccc4-5)cc3)cc21 ; CC1(C)c2ccccc2-c2ccc(-c3ccc(N(c4ccc(-c5ccc6ccccc6c5)cc4)c4ccc5c(c4)C(C)(C)c4ccccc4-5)cc3)cc21 ; CC1(C)c2ccccc2-c2ccc(-c3ccc(N(c4ccc(-c5ccc6ccccc6c5)cc4)c4ccc5c(c4)C(C)(C)c4ccccc4-5)cc3)cc21 ; CC1(C)c2ccccc2-c2ccc(-c3ccc(N(c4ccc(-c5ccc6ccccc6c5)cc4)c4cccc5c4-c4ccccc4C5(C)C)cc3)cc21]

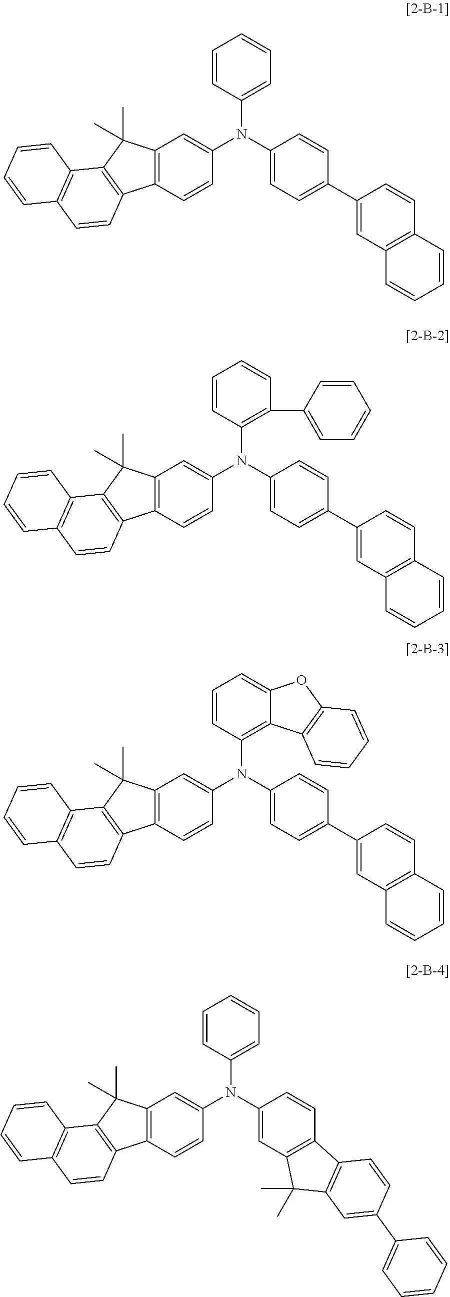 [SMILES: CC1(C)c2cc(N(c3ccccc3)c3ccc(-c4ccc5ccccc5c4)cc3)ccc2-c2ccc3ccccc3c21 ; CC1(C)c2cc(N(c3ccc(-c4ccc5ccccc5c4)cc3)c3ccccc3-c3ccccc3)ccc2-c2ccc3ccccc3c21 ; CC1(C)c2cc(N(c3ccc(-c4ccc5ccccc5c4)cc3)c3cccc4oc5ccccc5c34)ccc2-c2ccc3ccccc3c21 ; CC1(C)c2cc(-c3ccccc3)ccc2-c2ccc(N(c3ccccc3)c3ccc4c(c3)C(C)(C)c3c-4ccc4ccccc34)cc21]

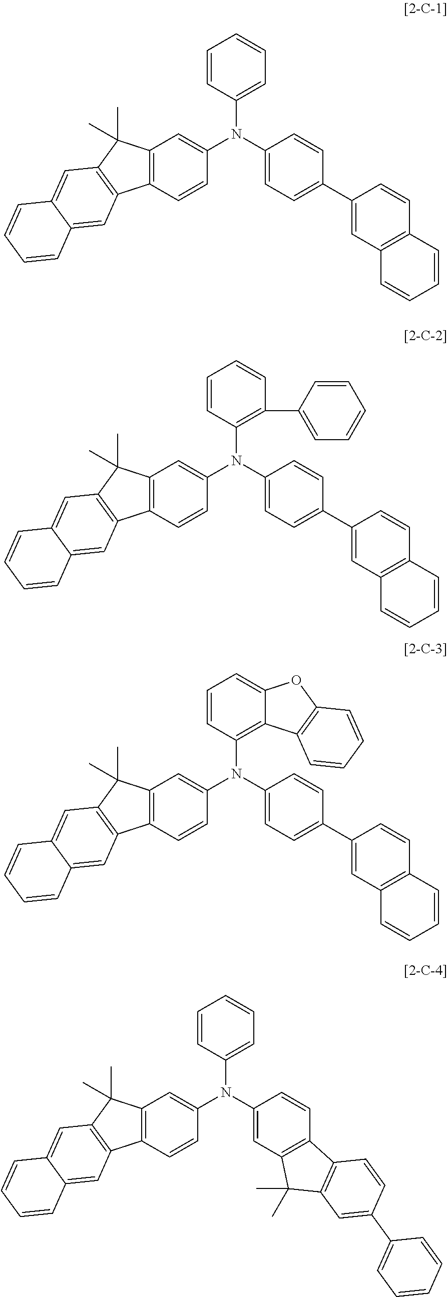 [SMILES: CC1(C)c2cc(N(c3ccccc3)c3ccc(-c4ccc5ccccc5c4)cc3)ccc2-c2cc3ccccc3cc21 ; CC1(C)c2cc(N(c3ccc(-c4ccc5ccccc5c4)cc3)c3ccccc3-c3ccccc3)ccc2-c2cc3ccccc3cc21 ; CC1(C)c2cc(N(c3ccc(-c4ccc5ccccc5c4)cc3)c3cccc4oc5ccccc5c34)ccc2-c2cc3ccccc3cc21 ; CC1(C)c2cc(-c3ccccc3)ccc2-c2ccc(N(c3ccccc3)c3ccc4c(c3)C(C)(C)c3cc5ccccc5cc3-4)cc21]

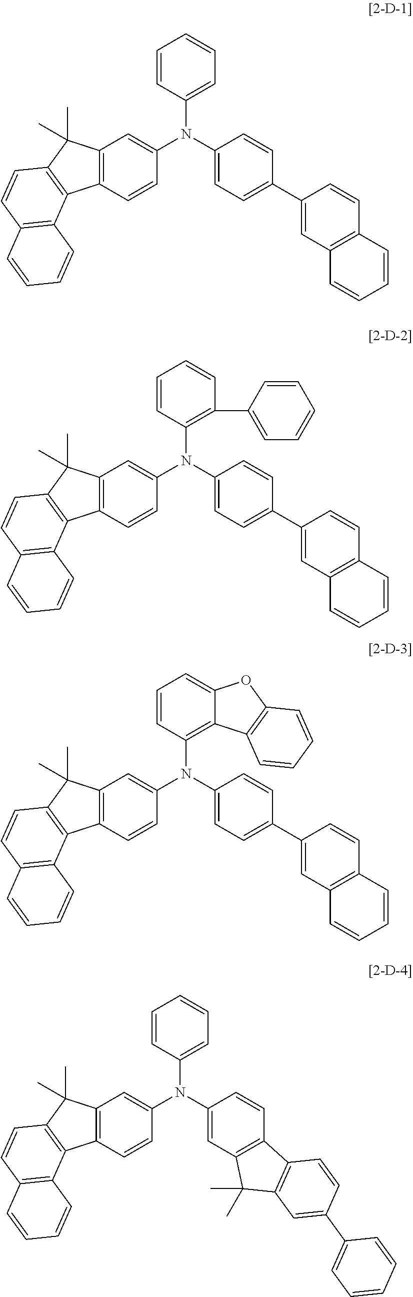 [SMILES: CC1(C)c2cc(N(c3ccccc3)c3ccc(-c4ccc5ccccc5c4)cc3)ccc2-c2c1ccc1ccccc21 ; CC1(C)c2cc(N(c3ccc(-c4ccc5ccccc5c4)cc3)c3ccccc3-c3ccccc3)ccc2-c2c1ccc1ccccc21 ; CC1(C)c2cc(N(c3ccc(-c4ccc5ccccc5c4)cc3)c3cccc4oc5ccccc5c34)ccc2-c2c1ccc1ccccc21 ; CC1(C)c2cc(-c3ccccc3)ccc2-c2ccc(N(c3ccccc3)c3ccc4c(c3)C(C)(C)c3ccc5ccccc5c3-4)cc21]

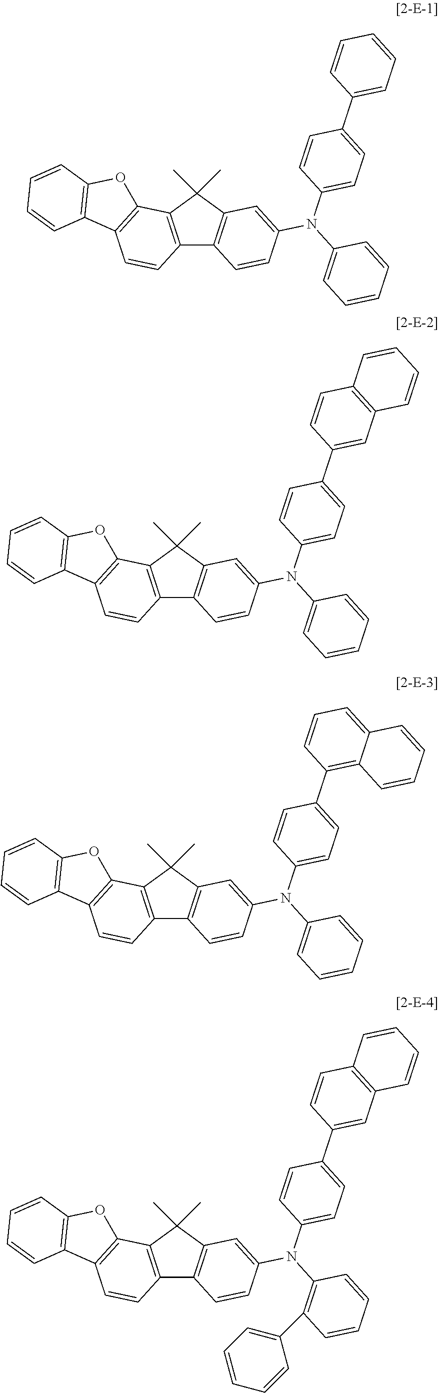 [SMILES: CC1(C)c2cc(N(c3ccccc3)c3ccc(-c4ccccc4)cc3)ccc2-c2ccc3c(oc4ccccc43)c21 ; CC1(C)c2cc(N(c3ccccc3)c3ccc(-c4ccc5ccccc5c4)cc3)ccc2-c2ccc3c(oc4ccccc43)c21 ; CC1(C)c2cc(N(c3ccccc3)c3ccc(-c4cccc5ccccc45)cc3)ccc2-c2ccc3c(oc4ccccc43)c21 ; CC1(C)c2cc(N(c3ccc(-c4ccc5ccccc5c4)cc3)c3ccccc3-c3ccccc3)ccc2-c2ccc3c(oc4ccccc43)c21]

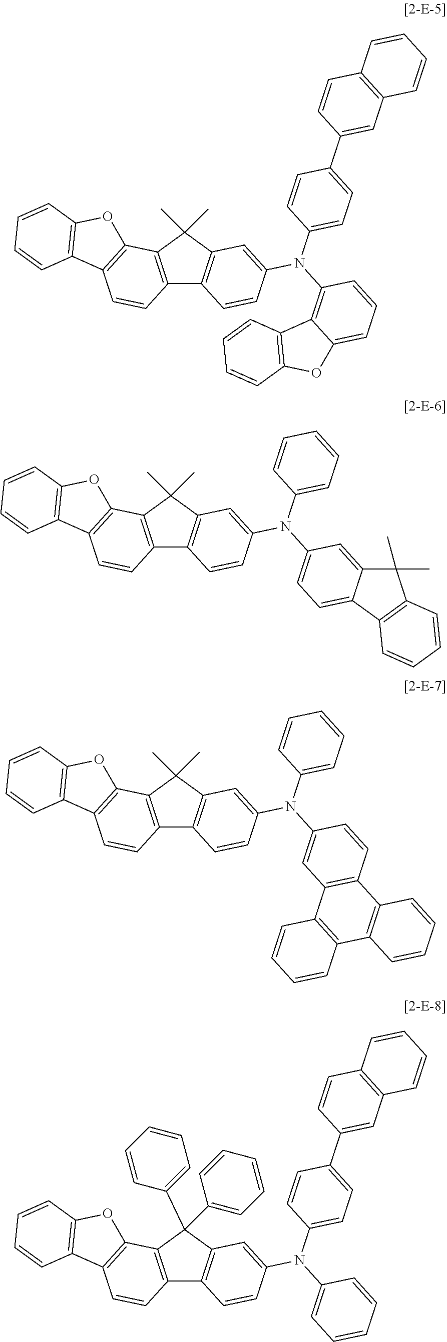 [SMILES: CC1(C)c2cc(N(c3ccc(-c4ccc5ccccc5c4)cc3)c3cccc4oc5ccccc5c34)ccc2-c2ccc3c(oc4ccccc43)c21 ; CC1(C)c2ccccc2-c2ccc(N(c3ccccc3)c3ccc4c(c3)C(C)(C)c3c-4ccc4c3oc3ccccc34)cc21 ; CC1(C)c2cc(N(c3ccccc3)c3ccc4c5ccccc5c5ccccc5c4c3)ccc2-c2ccc3c(oc4ccccc43)c21 ; c1ccc(N(c2ccc(-c3ccc4ccccc4c3)cc2)c2ccc3c(c2)C(c2ccccc2)(c2ccccc2)c2c-3ccc3c2oc2ccccc23)cc1]

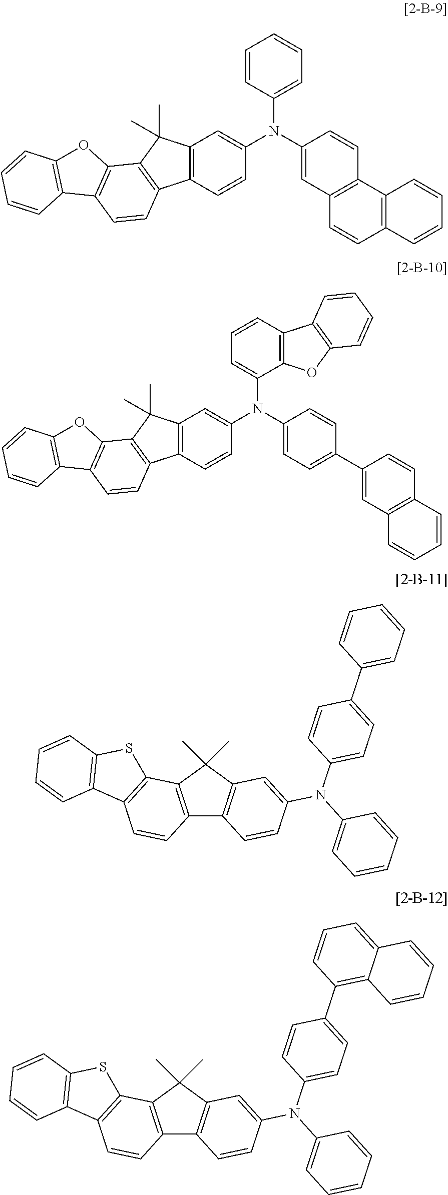 [SMILES: CC1(C)c2cc(N(c3ccccc3)c3ccc4c(ccc5ccccc54)c3)ccc2-c2ccc3c(oc4ccccc43)c21 ; CC1(C)c2cc(N(c3ccc(-c4ccc5ccccc5c4)cc3)c3cccc4c3oc3ccccc34)ccc2-c2ccc3c(oc4ccccc43)c21 ; CC1(C)c2cc(N(c3ccccc3)c3ccc(-c4ccccc4)cc3)ccc2-c2ccc3c(sc4ccccc43)c21 ; CC1(C)c2cc(N(c3ccccc3)c3ccc(-c4cccc5ccccc45)cc3)ccc2-c2ccc3c(sc4ccccc43)c21]

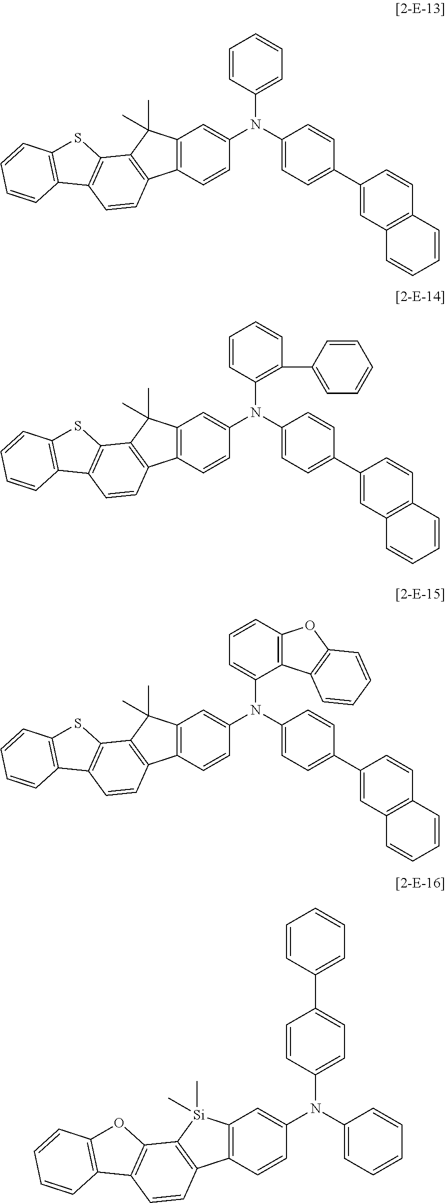 [SMILES: CC1(C)c2cc(N(c3ccccc3)c3ccc(-c4ccc5ccccc5c4)cc3)ccc2-c2ccc3c(sc4ccccc43)c21 ; CC1(C)c2cc(N(c3ccc(-c4ccc5ccccc5c4)cc3)c3ccccc3-c3ccccc3)ccc2-c2ccc3c(sc4ccccc43)c21 ; CC1(C)c2cc(N(c3ccc(-c4ccc5ccccc5c4)cc3)c3cccc4oc5ccccc5c34)ccc2-c2ccc3c(sc4ccccc43)c21 ; C[Si]1(C)c2cc(N(c3ccccc3)c3ccc(-c4ccccc4)cc3)ccc2-c2ccc3c(oc4ccccc43)c21]

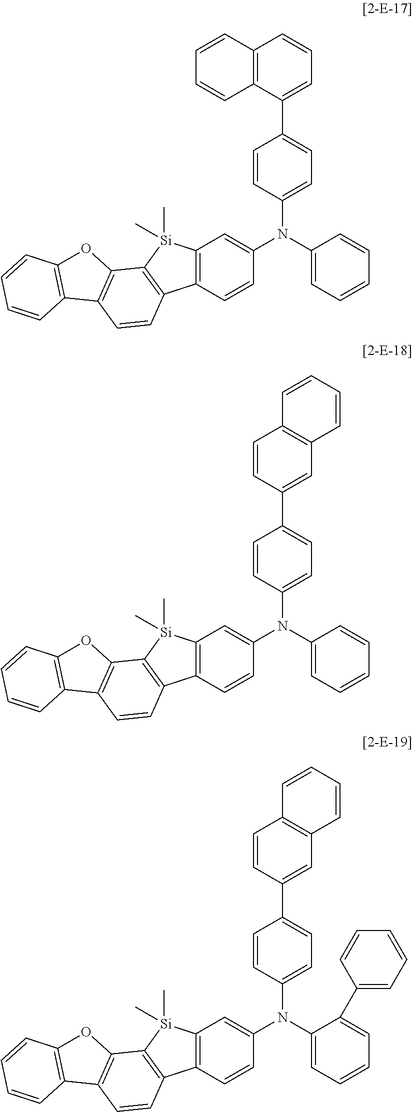 [SMILES: C[Si]1(C)c2cc(N(c3ccccc3)c3ccc(-c4cccc5ccccc45)cc3)ccc2-c2ccc3c(oc4ccccc43)c21 ; C[Si]1(C)c2cc(N(c3ccccc3)c3ccc(-c4ccc5ccccc5c4)cc3)ccc2-c2ccc3c(oc4ccccc43)c21 ; C[Si]1(C)c2cc(N(c3ccc(-c4ccc5ccccc5c4)cc3)c3ccccc3-c3ccccc3)ccc2-c2ccc3c(oc4ccccc43)c21]

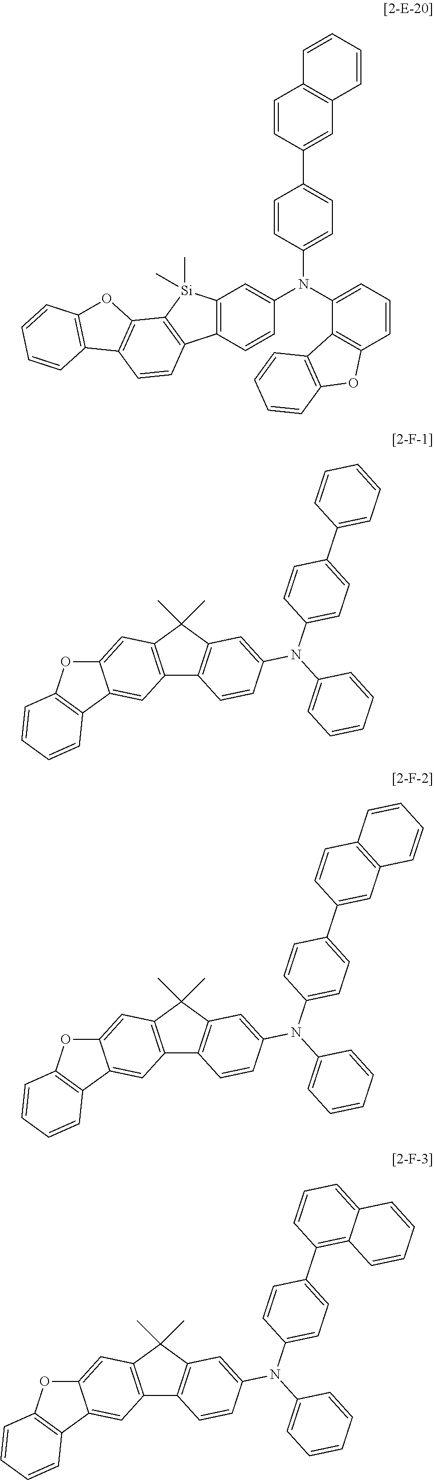 [SMILES: C[Si]1(C)c2cc(N(c3ccc(-c4ccc5ccccc5c4)cc3)c3cccc4oc5ccccc5c34)ccc2-c2ccc3c(oc4ccccc43)c21 ; CC1(C)c2cc(N(c3ccccc3)c3ccc(-c4ccccc4)cc3)ccc2-c2cc3c(cc21)oc1ccccc13 ; CC1(C)c2cc(N(c3ccccc3)c3ccc(-c4ccc5ccccc5c4)cc3)ccc2-c2cc3c(cc21)oc1ccccc13 ; CC1(C)c2cc(N(c3ccccc3)c3ccc(-c4cccc5ccccc45)cc3)ccc2-c2cc3c(cc21)oc1ccccc13]

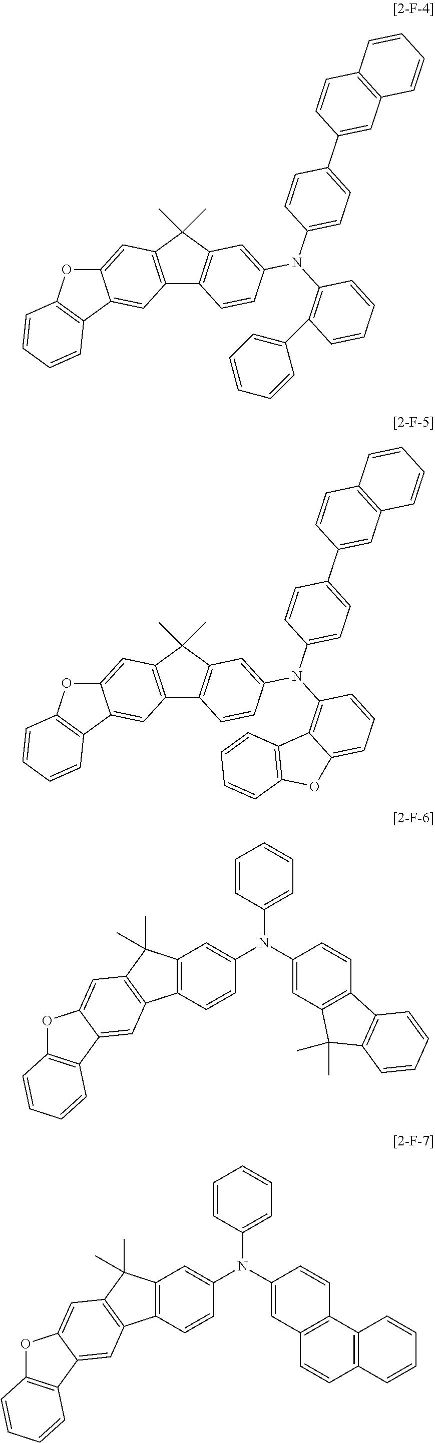

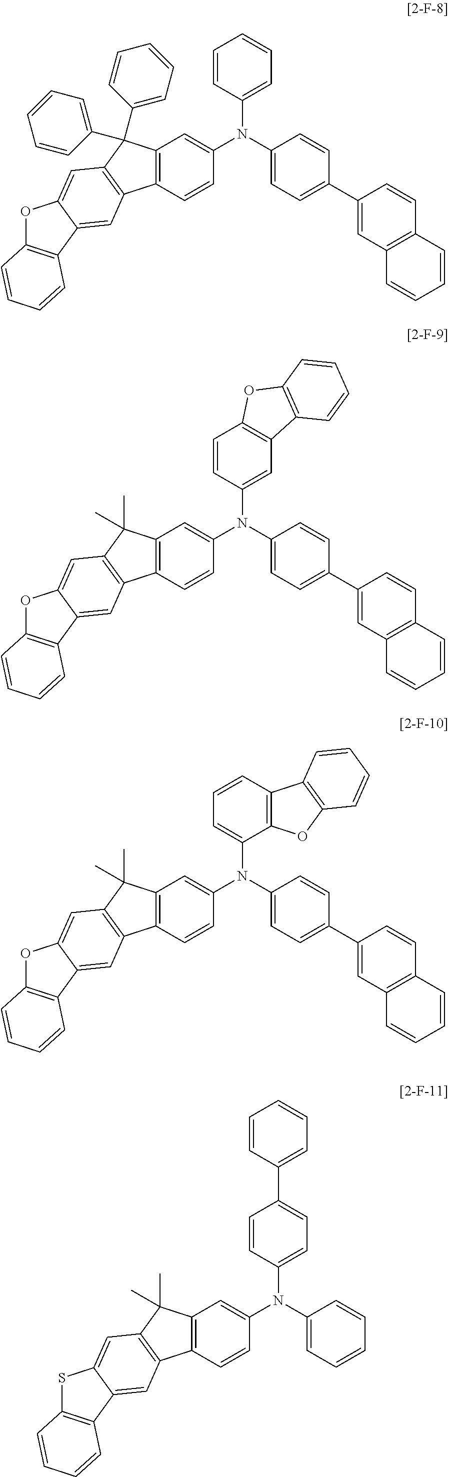 [SMILES: c1ccc(N(c2ccc(-c3ccc4ccccc4c3)cc2)c2ccc3c(c2)C(c2ccccc2)(c2ccccc2)c2cc4oc5ccccc5c4cc2-3)cc1 ; CC1(C)c2cc(N(c3ccc(-c4ccc5ccccc5c4)cc3)c3ccc4oc5ccccc5c4c3)ccc2-c2cc3c(cc21)oc1ccccc13 ; CC1(C)c2cc(N(c3ccc(-c4ccc5ccccc5c4)cc3)c3cccc4c3oc3ccccc34)ccc2-c2cc3c(cc21)oc1ccccc13 ; CC1(C)c2cc(N(c3ccccc3)c3ccc(-c4ccccc4)cc3)ccc2-c2cc3c(cc21)sc1ccccc13]

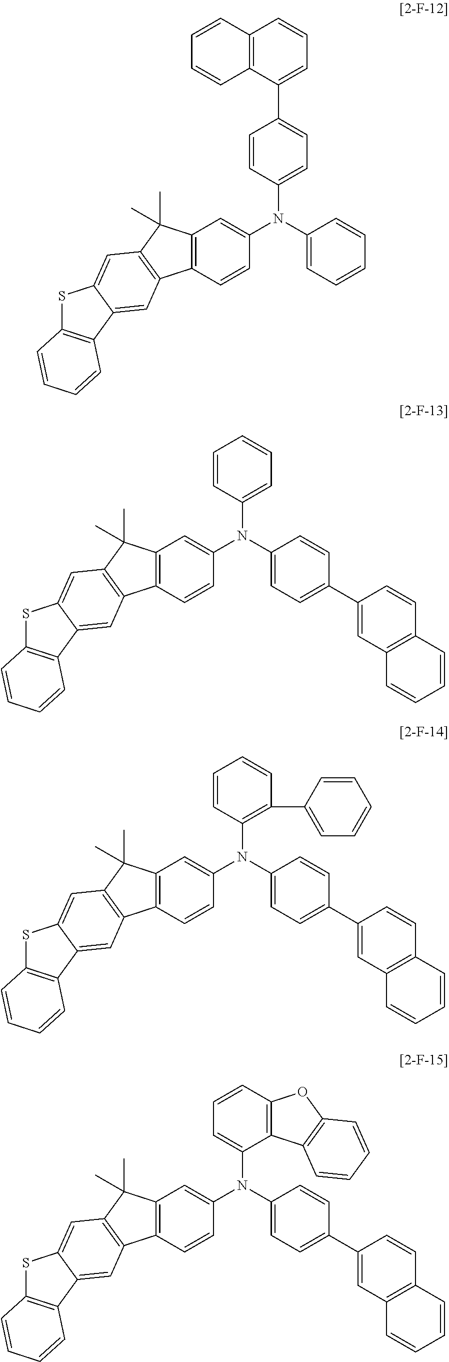 [SMILES: CC1(C)c2cc(N(c3ccccc3)c3ccc(-c4cccc5ccccc45)cc3)ccc2-c2cc3c(cc21)sc1ccccc13 ; CC1(C)c2cc(N(c3ccccc3)c3ccc(-c4ccc5ccccc5c4)cc3)ccc2-c2cc3c(cc21)sc1ccccc13 ; CC1(C)c2cc(N(c3ccc(-c4ccc5ccccc5c4)cc3)c3ccccc3-c3ccccc3)ccc2-c2cc3c(cc21)sc1ccccc13 ; CC1(C)c2cc(N(c3ccc(-c4ccc5ccccc5c4)cc3)c3cccc4oc5ccccc5c34)ccc2-c2cc3c(cc21)sc1ccccc13]

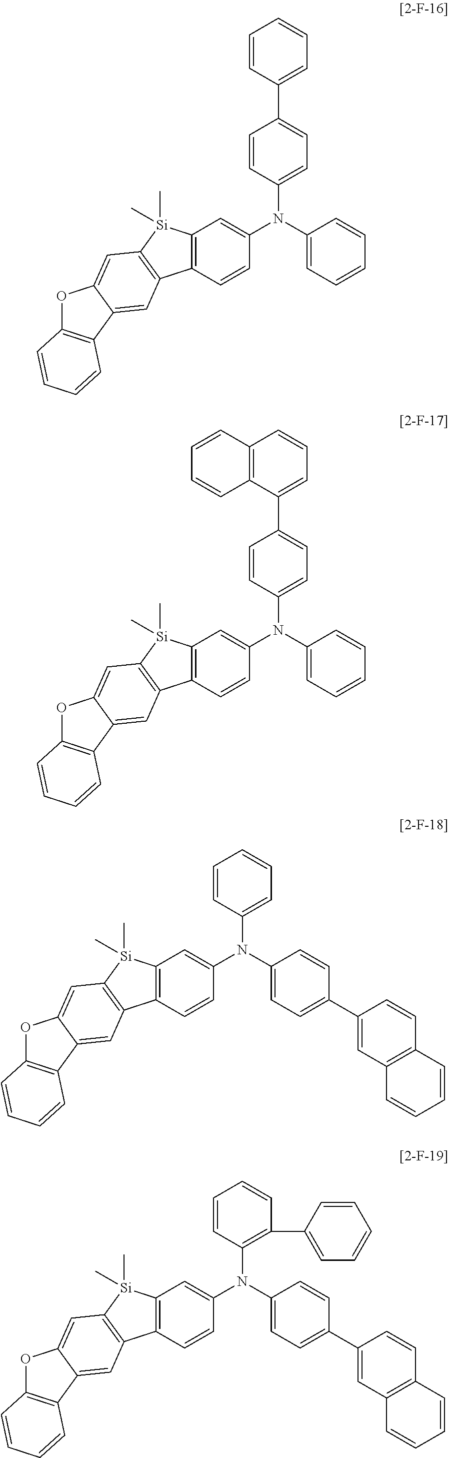 [SMILES: C[Si]1(C)c2cc(N(c3ccccc3)c3ccc(-c4ccccc4)cc3)ccc2-c2cc3c(cc21)oc1ccccc13 ; C[Si]1(C)c2cc(N(c3ccccc3)c3ccc(-c4cccc5ccccc45)cc3)ccc2-c2cc3c(cc21)oc1ccccc13 ; C[Si]1(C)c2cc(N(c3ccccc3)c3ccc(-c4ccc5ccccc5c4)cc3)ccc2-c2cc3c(cc21)oc1ccccc13 ; C[Si]1(C)c2cc(N(c3ccc(-c4ccc5ccccc5c4)cc3)c3ccccc3-c3ccccc3)ccc2-c2cc3c(cc21)oc1ccccc13]

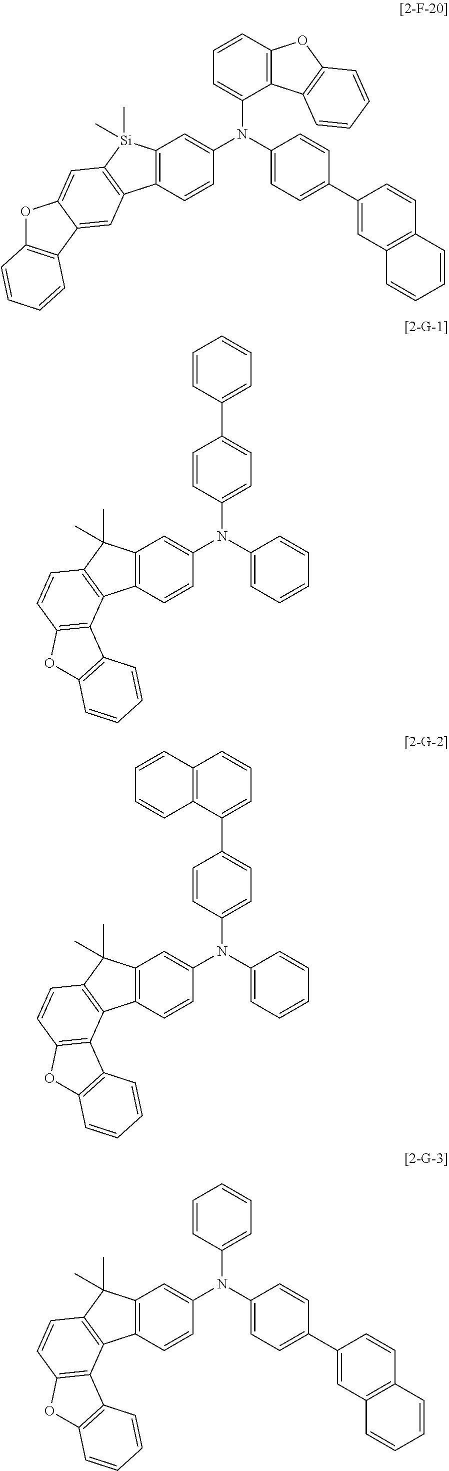 [SMILES: C[Si]1(C)c2cc(N(c3ccc(-c4ccc5ccccc5c4)cc3)c3cccc4oc5ccccc5c34)ccc2-c2cc3c(cc21)oc1ccccc13 ; CC1(C)c2cc(N(c3ccccc3)c3ccc(-c4ccccc4)cc3)ccc2-c2c1ccc1oc3ccccc3c21 ; CC1(C)c2cc(N(c3ccccc3)c3ccc(-c4cccc5ccccc45)cc3)ccc2-c2c1ccc1oc3ccccc3c21 ; CC1(C)c2cc(N(c3ccccc3)c3ccc(-c4ccc5ccccc5c4)cc3)ccc2-c2c1ccc1oc3ccccc3c21]

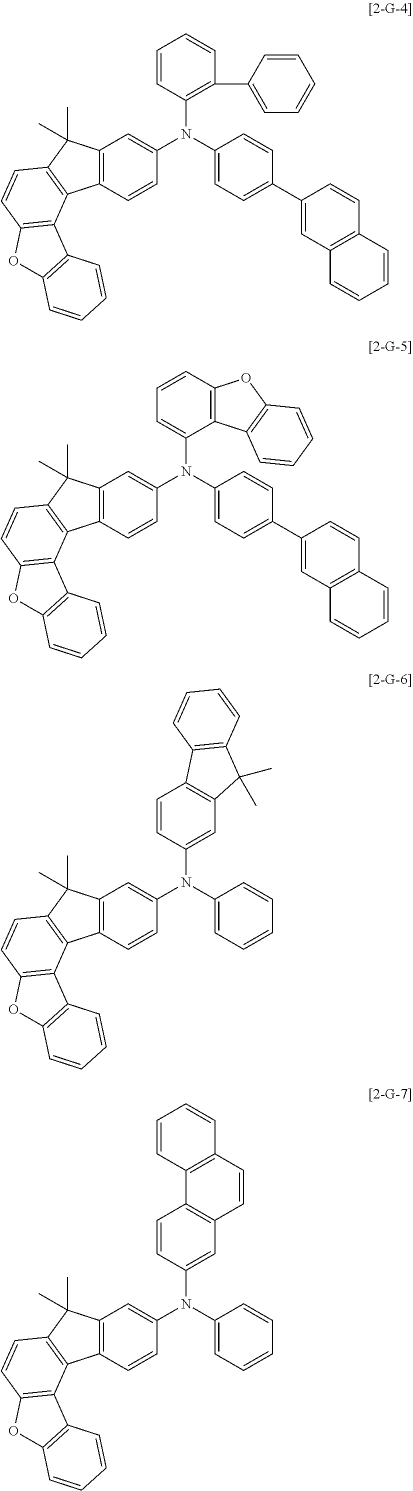 [SMILES: CC1(C)c2cc(N(c3ccc(-c4ccc5ccccc5c4)cc3)c3ccccc3-c3ccccc3)ccc2-c2c1ccc1oc3ccccc3c21 ; CC1(C)c2cc(N(c3ccc(-c4ccc5ccccc5c4)cc3)c3cccc4oc5ccccc5c34)ccc2-c2c1ccc1oc3ccccc3c21 ; CC1(C)c2ccccc2-c2ccc(N(c3ccccc3)c3ccc4c(c3)C(C)(C)c3ccc5oc6ccccc6c5c3-4)cc21 ; CC1(C)c2cc(N(c3ccccc3)c3ccc4c(ccc5ccccc54)c3)ccc2-c2c1ccc1oc3ccccc3c21]

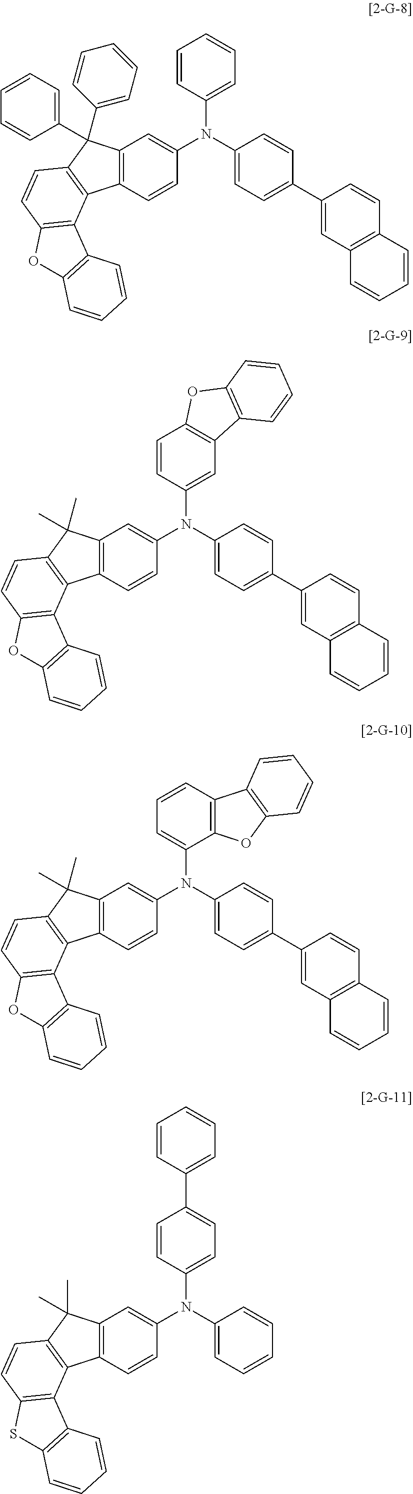 [SMILES: c1ccc(N(c2ccc(-c3ccc4ccccc4c3)cc2)c2ccc3c(c2)C(c2ccccc2)(c2ccccc2)c2ccc4oc5ccccc5c4c2-3)cc1 ; CC1(C)c2cc(N(c3ccc(-c4ccc5ccccc5c4)cc3)c3ccc4oc5ccccc5c4c3)ccc2-c2c1ccc1oc3ccccc3c21 ; CC1(C)c2cc(N(c3ccc(-c4ccc5ccccc5c4)cc3)c3cccc4c3oc3ccccc34)ccc2-c2c1ccc1oc3ccccc3c21 ; CC1(C)c2cc(N(c3ccccc3)c3ccc(-c4ccccc4)cc3)ccc2-c2c1ccc1sc3ccccc3c21]

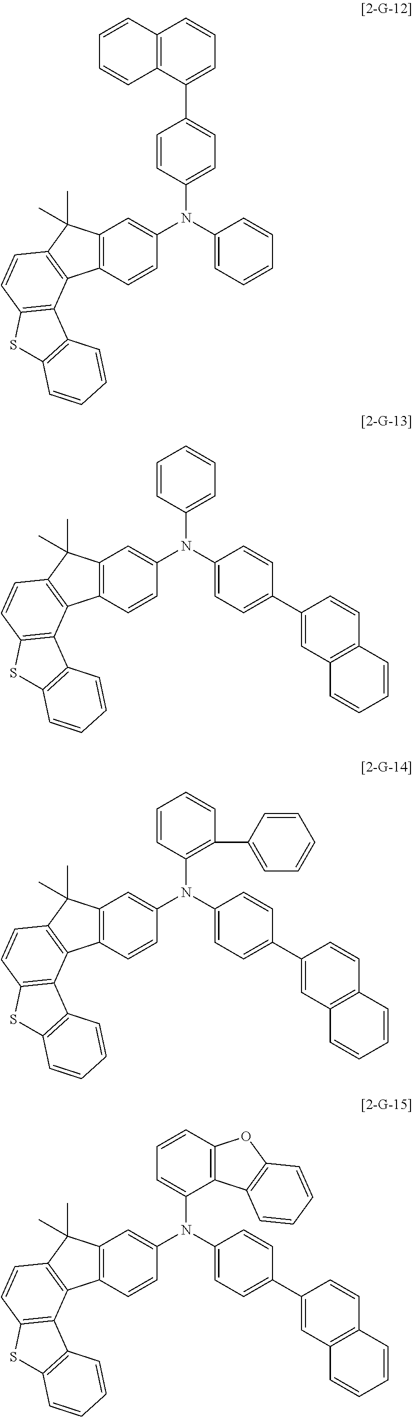 [SMILES: CC1(C)c2cc(N(c3ccccc3)c3ccc(-c4cccc5ccccc45)cc3)ccc2-c2c1ccc1sc3ccccc3c21 ; CC1(C)c2cc(N(c3ccccc3)c3ccc(-c4ccc5ccccc5c4)cc3)ccc2-c2c1ccc1sc3ccccc3c21 ; CC1(C)c2cc(N(c3ccc(-c4ccc5ccccc5c4)cc3)c3ccccc3-c3ccccc3)ccc2-c2c1ccc1sc3ccccc3c21 ; CC1(C)c2cc(N(c3ccc(-c4ccc5ccccc5c4)cc3)c3cccc4oc5ccccc5c34)ccc2-c2c1ccc1sc3ccccc3c21]

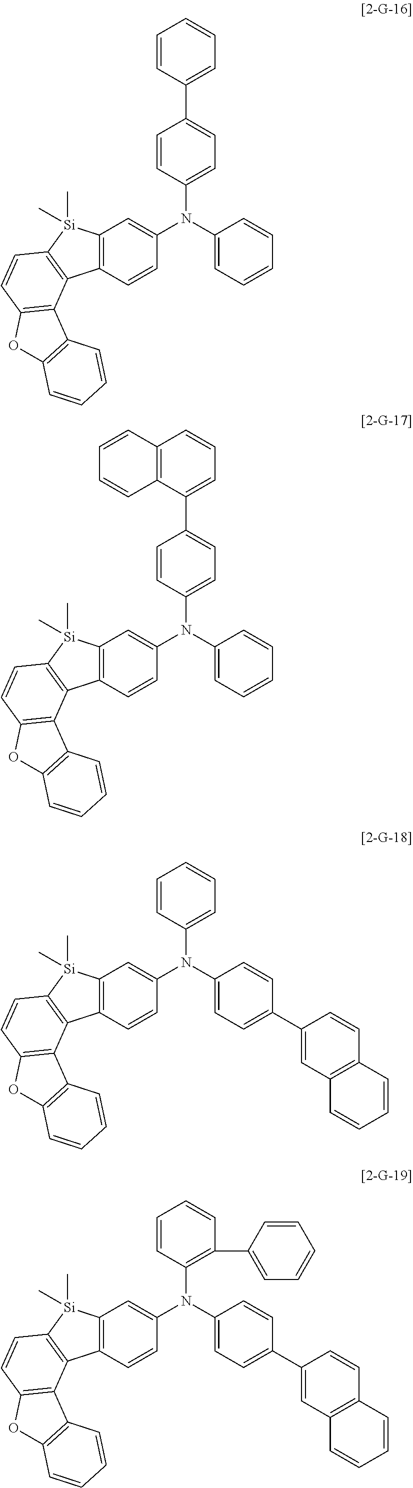

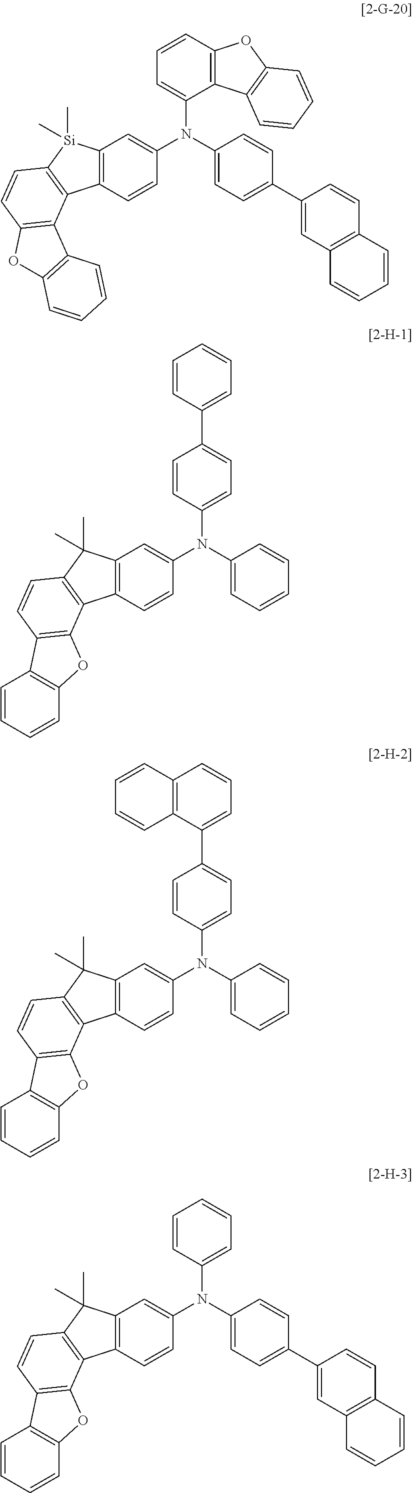 [SMILES: C[Si]1(C)c2cc(N(c3ccc(-c4ccc5ccccc5c4)cc3)c3cccc4oc5ccccc5c34)ccc2-c2c1ccc1oc3ccccc3c21 ; CC1(C)c2cc(N(c3ccccc3)c3ccc(-c4ccccc4)cc3)ccc2-c2c1ccc1c2oc2ccccc21 ; CC1(C)c2cc(N(c3ccccc3)c3ccc(-c4cccc5ccccc45)cc3)ccc2-c2c1ccc1c2oc2ccccc21 ; CC1(C)c2cc(N(c3ccccc3)c3ccc(-c4ccc5ccccc5c4)cc3)ccc2-c2c1ccc1c2oc2ccccc21]

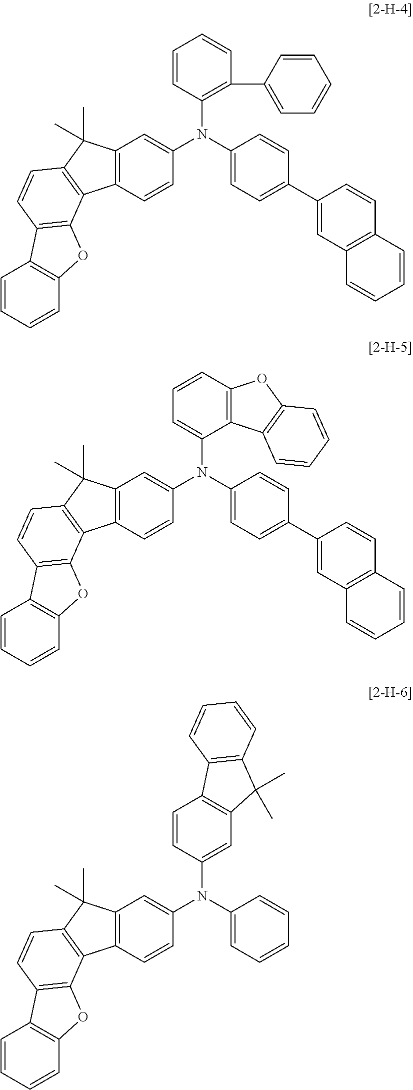 [SMILES: CC1(C)c2cc(N(c3ccc(-c4ccc5ccccc5c4)cc3)c3ccccc3-c3ccccc3)ccc2-c2c1ccc1c2oc2ccccc21 ; CC1(C)c2cc(N(c3ccc(-c4ccc5ccccc5c4)cc3)c3cccc4oc5ccccc5c34)ccc2-c2c1ccc1c2oc2ccccc21 ; CC1(C)c2ccccc2-c2ccc(N(c3ccccc3)c3ccc4c(c3)C(C)(C)c3ccc5c(oc6ccccc65)c3-4)cc21]

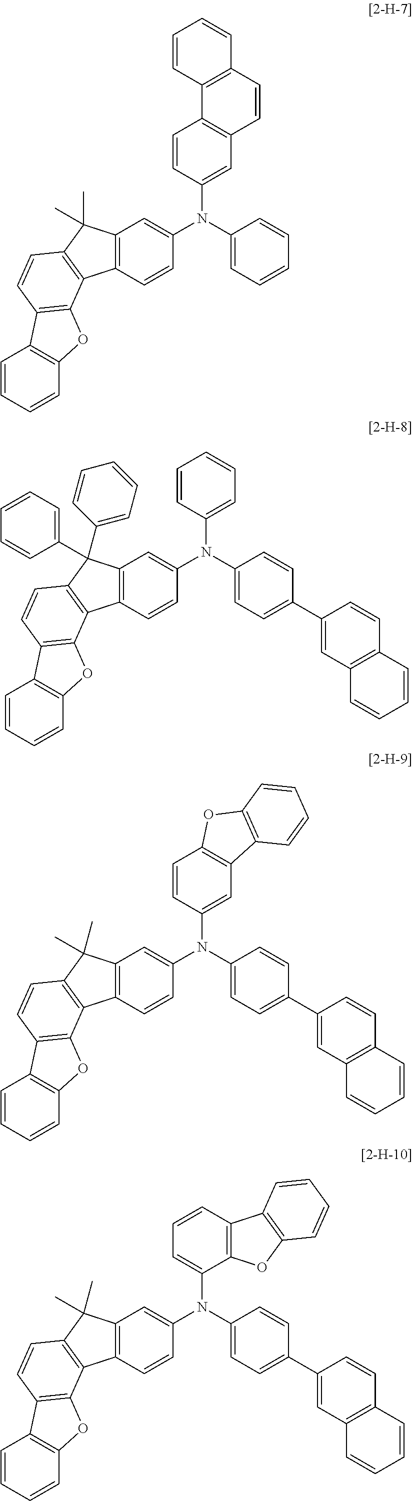 [SMILES: CC1(C)c2cc(N(c3ccccc3)c3ccc4c(ccc5ccccc54)c3)ccc2-c2c1ccc1c2oc2ccccc21 ; c1ccc(N(c2ccc(-c3ccc4ccccc4c3)cc2)c2ccc3c(c2)C(c2ccccc2)(c2ccccc2)c2ccc4c(oc5ccccc54)c2-3)cc1 ; CC1(C)c2cc(N(c3ccc(-c4ccc5ccccc5c4)cc3)c3ccc4oc5ccccc5c4c3)ccc2-c2c1ccc1c2oc2ccccc21 ; CC1(C)c2cc(N(c3ccc(-c4ccc5ccccc5c4)cc3)c3cccc4c3oc3ccccc34)ccc2-c2c1ccc1c2oc2ccccc21]

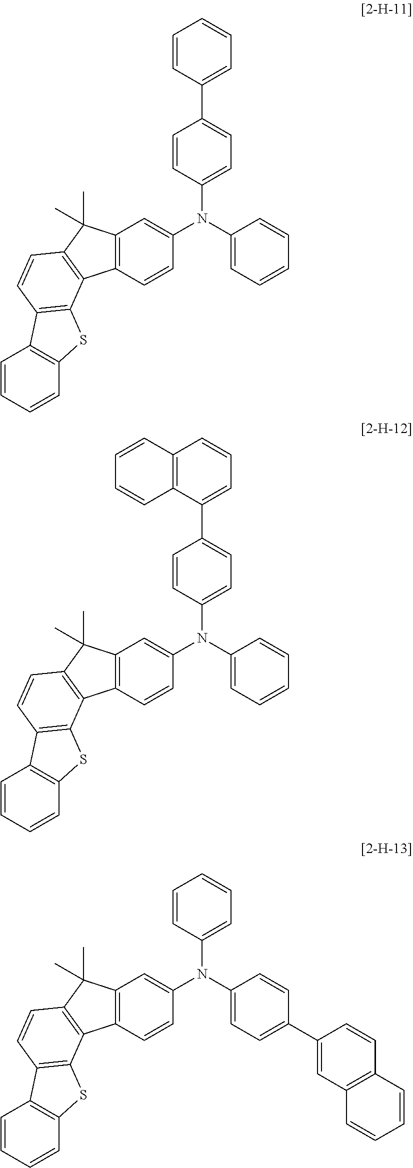 [SMILES: CC1(C)c2cc(N(c3ccccc3)c3ccc(-c4ccccc4)cc3)ccc2-c2c1ccc1c2sc2ccccc21 ; CC1(C)c2cc(N(c3ccccc3)c3ccc(-c4cccc5ccccc45)cc3)ccc2-c2c1ccc1c2sc2ccccc21 ; CC1(C)c2cc(N(c3ccccc3)c3ccc(-c4ccc5ccccc5c4)cc3)ccc2-c2c1ccc1c2sc2ccccc21]

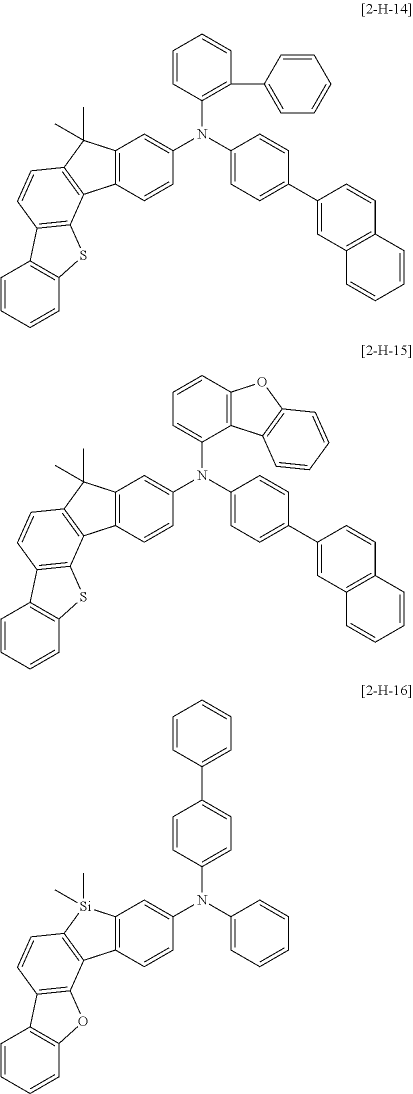 [SMILES: CC1(C)c2cc(N(c3ccc(-c4ccc5ccccc5c4)cc3)c3ccccc3-c3ccccc3)ccc2-c2c1ccc1c2sc2ccccc21 ; CC1(C)c2cc(N(c3ccc(-c4ccc5ccccc5c4)cc3)c3cccc4oc5ccccc5c34)ccc2-c2c1ccc1c2sc2ccccc21 ; C[Si]1(C)c2cc(N(c3ccccc3)c3ccc(-c4ccccc4)cc3)ccc2-c2c1ccc1c2oc2ccccc21]

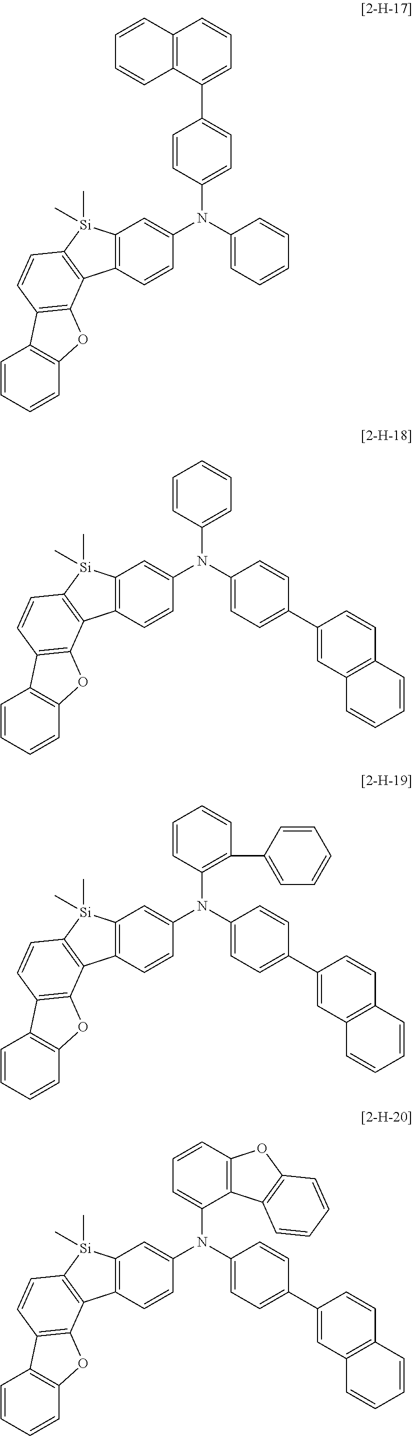 [SMILES: C[Si]1(C)c2cc(N(c3ccccc3)c3ccc(-c4cccc5ccccc45)cc3)ccc2-c2c1ccc1c2oc2ccccc21 ; C[Si]1(C)c2cc(N(c3ccccc3)c3ccc(-c4ccc5ccccc5c4)cc3)ccc2-c2c1ccc1c2oc2ccccc21 ; C[Si]1(C)c2cc(N(c3ccc(-c4ccc5ccccc5c4)cc3)c3ccccc3-c3ccccc3)ccc2-c2c1ccc1c2oc2ccccc21 ; C[Si]1(C)c2cc(N(c3ccc(-c4ccc5ccccc5c4)cc3)c3cccc4oc5ccccc5c34)ccc2-c2c1ccc1c2oc2ccccc21]

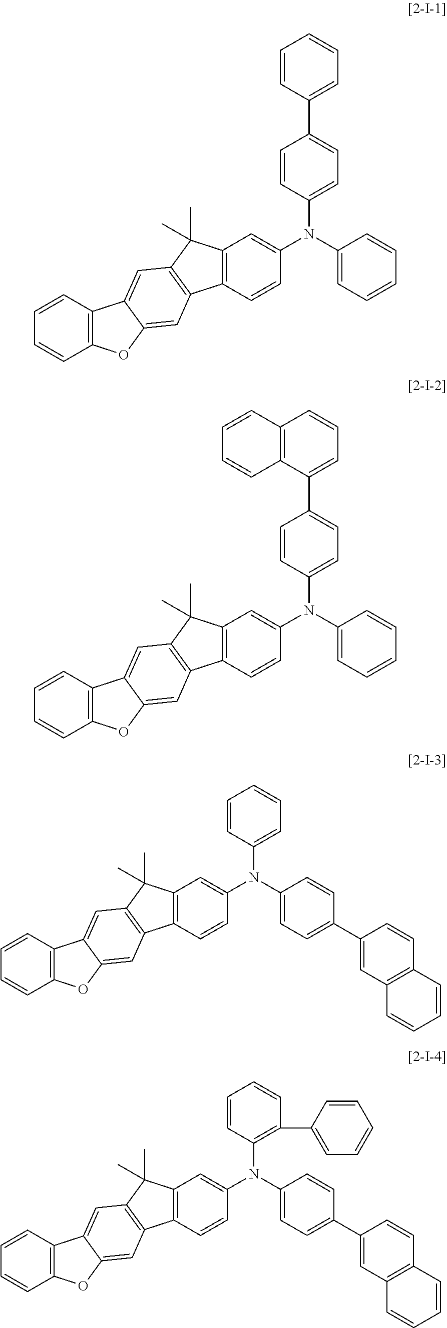 [SMILES: CC1(C)c2cc(N(c3ccccc3)c3ccc(-c4ccccc4)cc3)ccc2-c2cc3oc4ccccc4c3cc21 ; CC1(C)c2cc(N(c3ccccc3)c3ccc(-c4cccc5ccccc45)cc3)ccc2-c2cc3oc4ccccc4c3cc21 ; CC1(C)c2cc(N(c3ccccc3)c3ccc(-c4ccc5ccccc5c4)cc3)ccc2-c2cc3oc4ccccc4c3cc21 ; CC1(C)c2cc(N(c3ccc(-c4ccc5ccccc5c4)cc3)c3ccccc3-c3ccccc3)ccc2-c2cc3oc4ccccc4c3cc21]

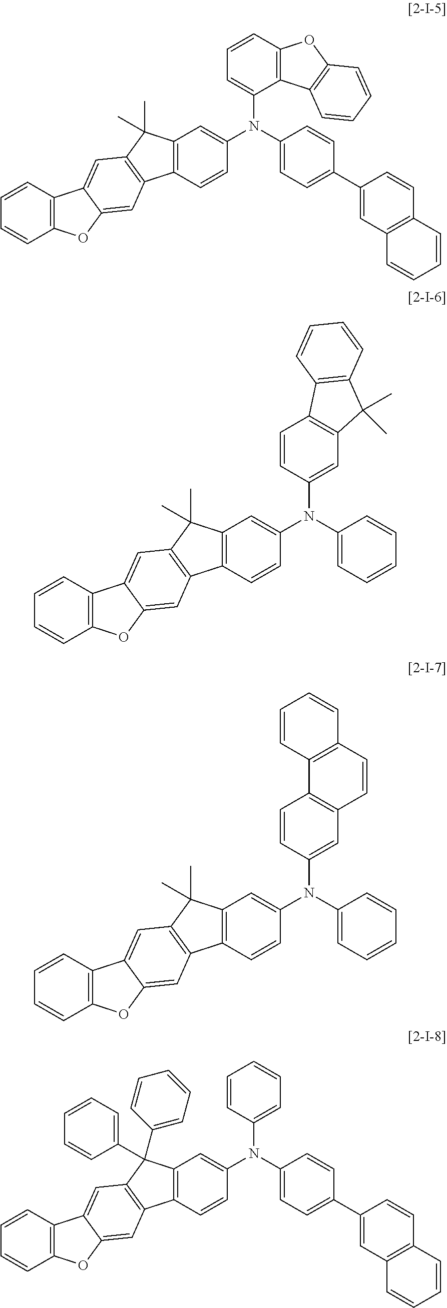 [SMILES: CC1(C)c2cc(N(c3ccc(-c4ccc5ccccc5c4)cc3)c3cccc4oc5ccccc5c34)ccc2-c2cc3oc4ccccc4c3cc21 ; CC1(C)c2ccccc2-c2ccc(N(c3ccccc3)c3ccc4c(c3)C(C)(C)c3cc5c(cc3-4)oc3ccccc35)cc21 ; CC1(C)c2cc(N(c3ccccc3)c3ccc4c(ccc5ccccc54)c3)ccc2-c2cc3oc4ccccc4c3cc21 ; c1ccc(N(c2ccc(-c3ccc4ccccc4c3)cc2)c2ccc3c(c2)C(c2ccccc2)(c2ccccc2)c2cc4c(cc2-3)oc2ccccc24)cc1]

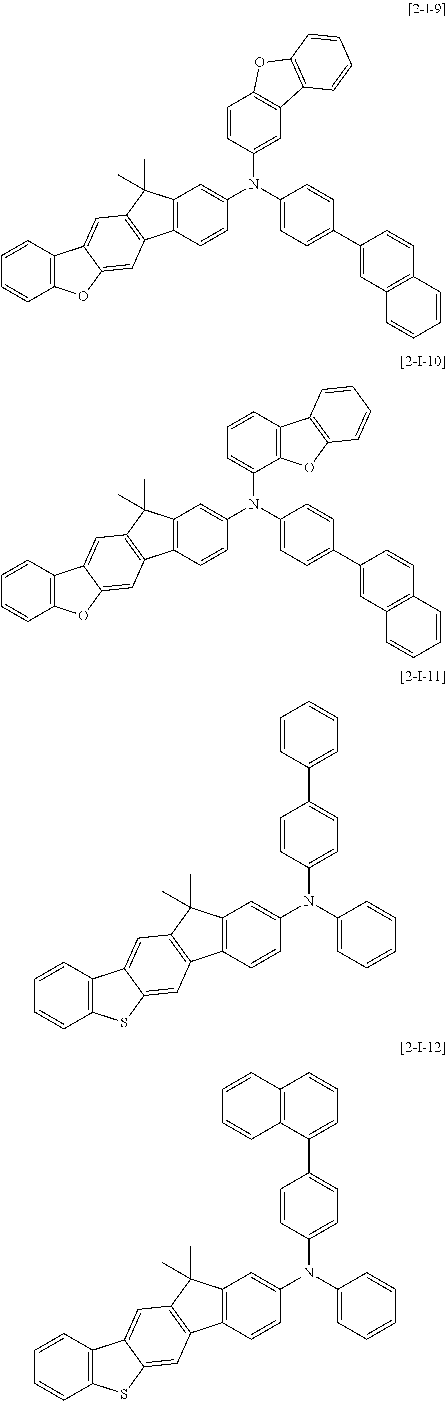 [SMILES: CC1(C)c2cc(N(c3ccc(-c4ccc5ccccc5c4)cc3)c3ccc4oc5ccccc5c4c3)ccc2-c2cc3oc4ccccc4c3cc21 ; CC1(C)c2cc(N(c3ccc(-c4ccc5ccccc5c4)cc3)c3cccc4c3oc3ccccc34)ccc2-c2cc3oc4ccccc4c3cc21 ; CC1(C)c2cc(N(c3ccccc3)c3ccc(-c4ccccc4)cc3)ccc2-c2cc3sc4ccccc4c3cc21 ; CC1(C)c2cc(N(c3ccccc3)c3ccc(-c4cccc5ccccc45)cc3)ccc2-c2cc3sc4ccccc4c3cc21]

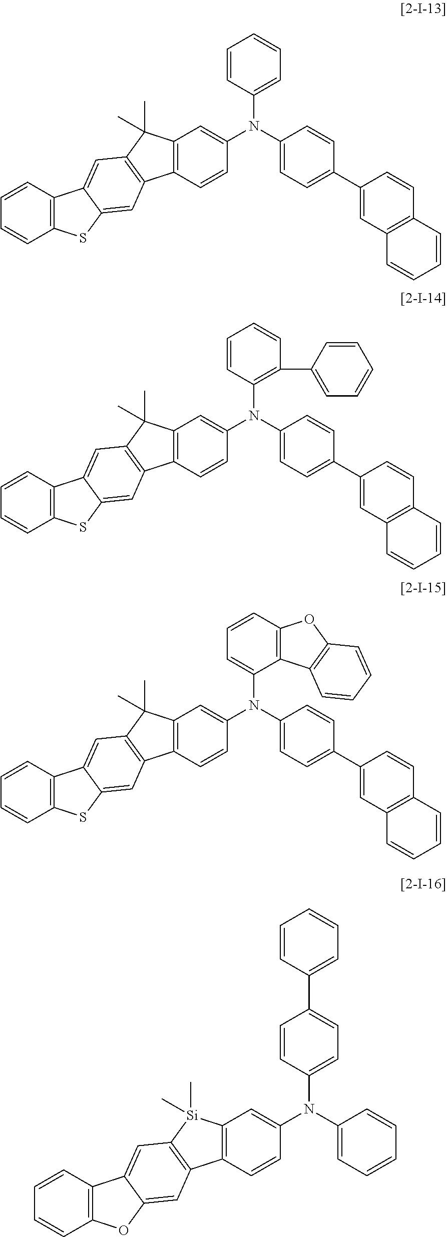 [SMILES: CC1(C)c2cc(N(c3ccccc3)c3ccc(-c4ccc5ccccc5c4)cc3)ccc2-c2cc3sc4ccccc4c3cc21 ; CC1(C)c2cc(N(c3ccc(-c4ccc5ccccc5c4)cc3)c3ccccc3-c3ccccc3)ccc2-c2cc3sc4ccccc4c3cc21 ; CC1(C)c2cc(N(c3ccc(-c4ccc5ccccc5c4)cc3)c3cccc4oc5ccccc5c34)ccc2-c2cc3sc4ccccc4c3cc21 ; C[Si]1(C)c2cc(N(c3ccccc3)c3ccc(-c4ccccc4)cc3)ccc2-c2cc3oc4ccccc4c3cc21]

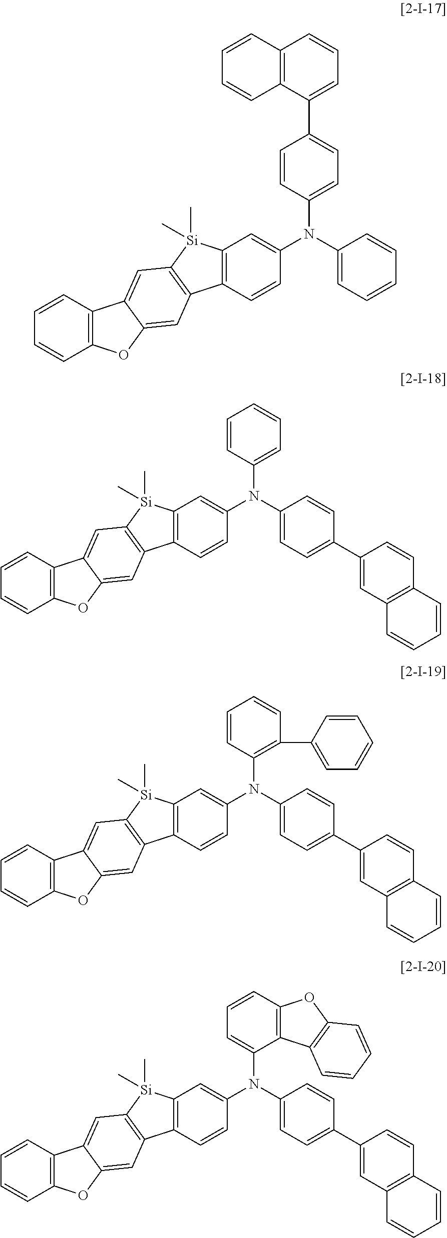 [SMILES: C[Si]1(C)c2cc(N(c3ccccc3)c3ccc(-c4cccc5ccccc45)cc3)ccc2-c2cc3oc4ccccc4c3cc21 ; C[Si]1(C)c2cc(N(c3ccccc3)c3ccc(-c4ccc5ccccc5c4)cc3)ccc2-c2cc3oc4ccccc4c3cc21 ; C[Si]1(C)c2cc(N(c3ccc(-c4ccc5ccccc5c4)cc3)c3ccccc3-c3ccccc3)ccc2-c2cc3oc4ccccc4c3cc21 ; C[Si]1(C)c2cc(N(c3ccc(-c4ccc5ccccc5c4)cc3)c3cccc4oc5ccccc5c34)ccc2-c2cc3oc4ccccc4c3cc21]

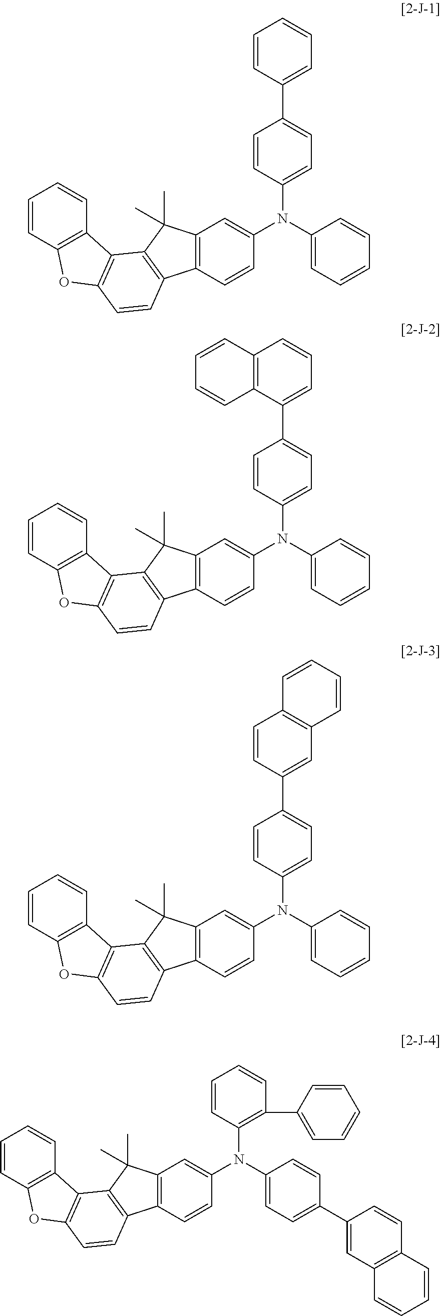 [SMILES: CC1(C)c2cc(N(c3ccccc3)c3ccc(-c4ccccc4)cc3)ccc2-c2ccc3oc4ccccc4c3c21 ; CC1(C)c2cc(N(c3ccccc3)c3ccc(-c4cccc5ccccc45)cc3)ccc2-c2ccc3oc4ccccc4c3c21 ; CC1(C)c2cc(N(c3ccccc3)c3ccc(-c4ccc5ccccc5c4)cc3)ccc2-c2ccc3oc4ccccc4c3c21 ; CC1(C)c2cc(N(c3ccc(-c4ccc5ccccc5c4)cc3)c3ccccc3-c3ccccc3)ccc2-c2ccc3oc4ccccc4c3c21]

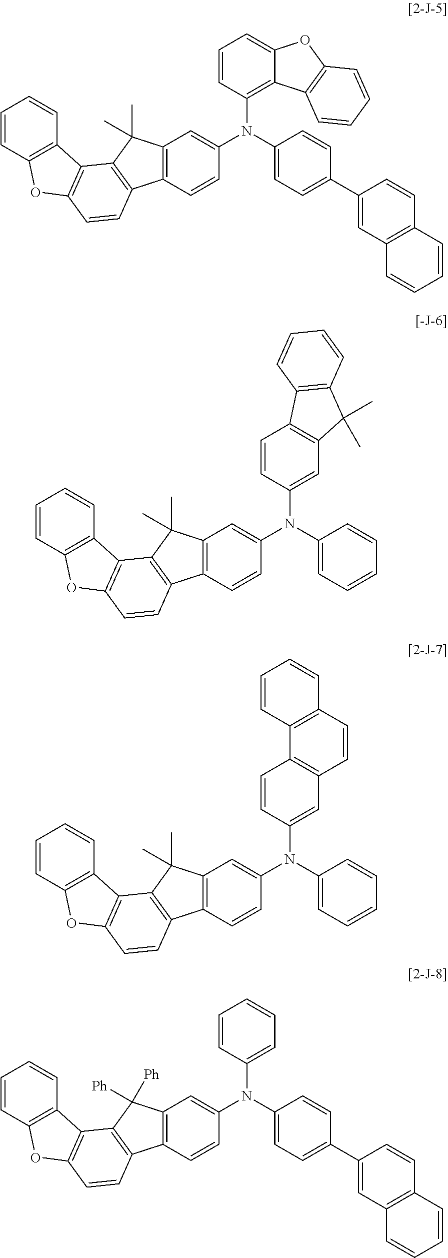 [SMILES: CC1(C)c2cc(N(c3ccc(-c4ccc5ccccc5c4)cc3)c3cccc4oc5ccccc5c34)ccc2-c2ccc3oc4ccccc4c3c21 ; CC1(C)c2ccccc2-c2ccc(N(c3ccccc3)c3ccc4c(c3)C(C)(C)c3c-4ccc4oc5ccccc5c34)cc21 ; CC1(C)c2cc(N(c3ccccc3)c3ccc4c(ccc5ccccc54)c3)ccc2-c2ccc3oc4ccccc4c3c21 ; c1ccc(N(c2ccc(-c3ccc4ccccc4c3)cc2)c2ccc3c(c2)C(c2ccccc2)(c2ccccc2)c2c-3ccc3oc4ccccc4c23)cc1]

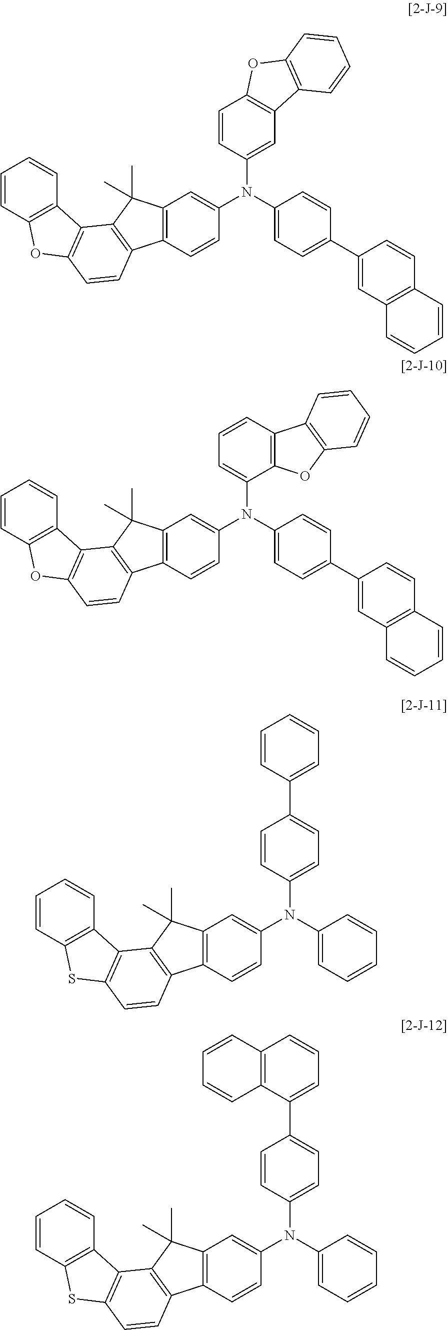 [SMILES: CC1(C)c2cc(N(c3ccc(-c4ccc5ccccc5c4)cc3)c3ccc4oc5ccccc5c4c3)ccc2-c2ccc3oc4ccccc4c3c21 ; CC1(C)c2cc(N(c3ccc(-c4ccc5ccccc5c4)cc3)c3cccc4c3oc3ccccc34)ccc2-c2ccc3oc4ccccc4c3c21 ; CC1(C)c2cc(N(c3ccccc3)c3ccc(-c4ccccc4)cc3)ccc2-c2ccc3sc4ccccc4c3c21 ; CC1(C)c2cc(N(c3ccccc3)c3ccc(-c4cccc5ccccc45)cc3)ccc2-c2ccc3sc4ccccc4c3c21]

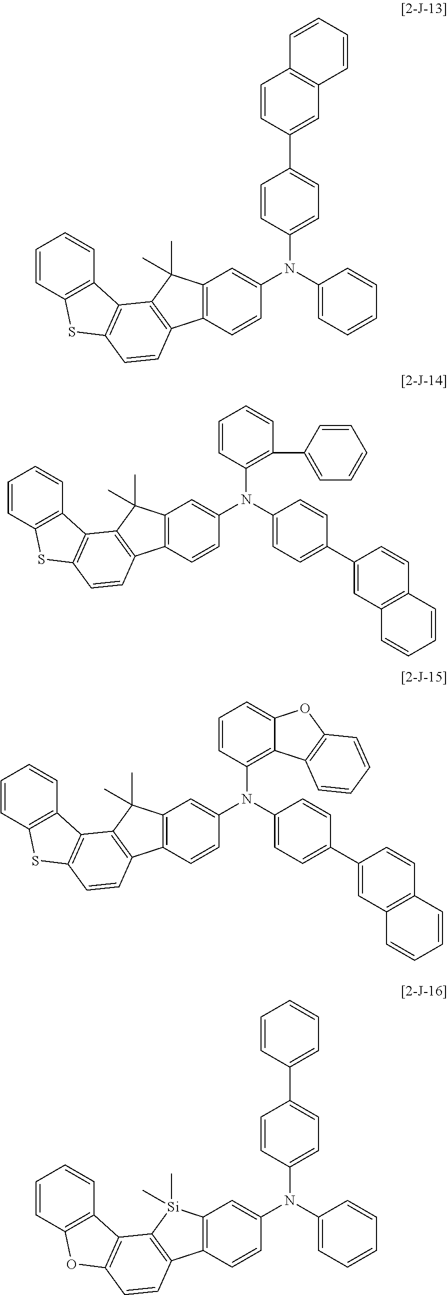 [SMILES: CC1(C)c2cc(N(c3ccccc3)c3ccc(-c4ccc5ccccc5c4)cc3)ccc2-c2ccc3sc4ccccc4c3c21 ; CC1(C)c2cc(N(c3ccc(-c4ccc5ccccc5c4)cc3)c3ccccc3-c3ccccc3)ccc2-c2ccc3sc4ccccc4c3c21 ; CC1(C)c2cc(N(c3ccc(-c4ccc5ccccc5c4)cc3)c3cccc4oc5ccccc5c34)ccc2-c2ccc3sc4ccccc4c3c21 ; C[Si]1(C)c2cc(N(c3ccccc3)c3ccc(-c4ccccc4)cc3)ccc2-c2ccc3oc4ccccc4c3c21]

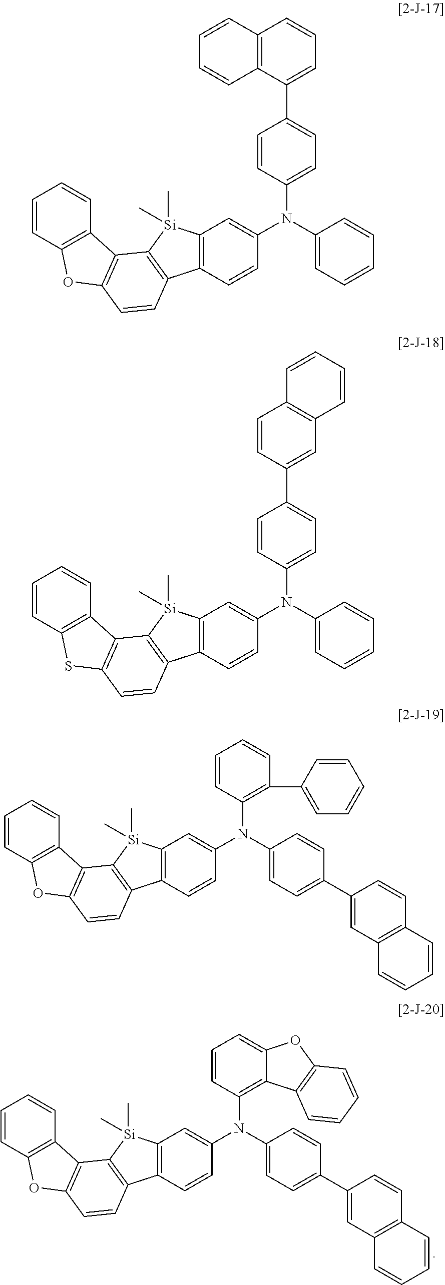 [SMILES: C[Si]1(C)c2cc(N(c3ccccc3)c3ccc(-c4cccc5ccccc45)cc3)ccc2-c2ccc3oc4ccccc4c3c21 ; C[Si]1(C)c2cc(N(c3ccccc3)c3ccc(-c4ccc5ccccc5c4)cc3)ccc2-c2ccc3sc4ccccc4c3c21 ; C[Si]1(C)c2cc(N(c3ccc(-c4ccc5ccccc5c4)cc3)c3ccccc3-c3ccccc3)ccc2-c2ccc3oc4ccccc4c3c21 ; C[Si]1(C)c2cc(N(c3ccc(-c4ccc5ccccc5c4)cc3)c3cccc4oc5ccccc5c34)ccc2-c2ccc3oc4ccccc4c3c21]

10. An organic optoelectronic device, comprising:
an anode and a cathode facing each other, and
at least one organic layer between the anode and the cathode,
wherein:
the at least one organic layer includes a light emitting layer, and
the light emitting layer includes the composition for an organic optoelectronic device as claimed in claim 1.

11. The organic optoelectronic device as claimed in claim 10, wherein the composition for an organic optoelectronic device is a host of the light emitting layer.

12. A display device comprising the organic optoelectronic device as claimed in claim 10.
